SEQUENCE LISTING (55 sequences)

SEQ ID NO: 1 - protein, 99 aa, Homo sapiens
MQPRVLLVVALLALLASARASEAEDASLLSFMQGYMKHATKTAKDALSSVQESQVAQQARGWVTDGFSSLKDYWSTVKDKFSEFWDLDPEVRPTSAVAA

SEQ ID NO: 2 - protein, 1474 aa, Homo sapiens
MGKNKLLHPSLVLLLLVLLPTDASVSGKPQYMVLVPSLLHTETTEKGCVLLSYLNETVTVSASLESVRGNRSLFTDLEAENDVLHCVAFAVPKSSSNEEVMFLTVQVKGPTQEFKKRTTVMVKNEDSLVFVQTDKSIYKPGQTVKFRVVSMDENFHPLNELIPLVYIQDPKGNRIAQWQSFQLEGGLKQFSFPLSSEPFQGSYKVVVQKKSGGRTEHPFTVEEFVLPKFEVQVTVPKIITILEEEMNVSVCGLYTYGKPVPGHVTVSICRKYSDASDCHGEDSQAFCEKFSGQLNSHGCFYQQVKTKVFQLKRKEYEMKLHTEAQIQEEGTVVELTGRQSSEITRTITKLSFVKVDSHFRQGIPFFGQVRLVDGKGVPIPNKVIFIRGNEANYYSNATTDEHGLVQFSINTTNVMGTSLTVRVNYKDRSPCYGYQWVSEEHEEAHHTAYLVFSPSKSFVHLEPMSHELPCGHTQTVQAHYILNGGTLLGLKKLSFYYLIMAKGGIVRTGTHGLLVKQEDMKGHFSISIPVKSDIAPVARLLIYAVLPTGDVIGDSAKYDVENCLANKVDLSFSPSQSLPASHAHLRVTAAPQSVCALRAVDQSVLLMKPDAELSASSVYNLLPEKDLTGFPGPLNDQDNEDCINRHNVYINGITYTPVSSTNEKDMYSFLEDMGLKAFTNSKIRKPKMCPQLQQYEMHGPEGLRVGFYESDVMGRGHARLVHVEEPHTETVRKYFPETWIWDLVVVNSAGVAEVGVTVPDTITEWKAGAFCLSEDAGLGISSTASLRAFQPFFVELTMPYSVIRGEAFTLKATVLNYLPKCIRVSVQLEASPAFLAVPVEKEQAPHCICANGRQTVSWAVTPKSLGNVNFTVSAEALESQELCGTEVPSVPEHGRKDTVIKPLLVEPEGLEKETTFNSLLCPSGGEVSEELSLKLPPNVVEESARASVSVLGDILGSAMQNTQNLLQMPYGCGEQNMVLFAPNIYVLDYLNETQQLTPEIKSKAIGYLNTGYQRQLNYKHYDGSYSTFGERYGRNQGNTWLTAFVLKTFAQARAYIFIDEAHITQALIWLSQRQKDNGCFRSSGSLLNNAIKGGVEDEVTLSAYITIALLEIPLTVTHPVVRNALFCLESAWKTAQEGDHGSHVYTKALLAYAFALAGNQDKRKEVLKSLNEEAVKKDNSVHWERPQKPKAPVGHFYEPQAPSAEVEMTSYVLLAYLTAQPAPTSEDLTSATNIVKWITKQQNAQGGFSSTQDTVVALHALSKYGAATFTRTGKAAQVTIQSSGTFSSKFQVDNNNRLLLQQVSLPELPGEYSMKVTGEGCVYLQTSLKYNILPEKEEFPFALGVQTLPQTCDEPKAHTSFQISLSVSYTGSRSASNMAIVDVKMVSGFIPLKPTVKMLERSNHVSRTEVSSNHVLIYLDKVSNQTLSLFFTVLQDVPVRDLKPAIVKVYDYYETDEFAIAEYNAPCSKDLGNA

SEQ ID NO: 3 - protein, 638 aa, Homo sapiens
MILFKQATYFISLFATVSCGCLTQLYENAFFRGGDVASMYTPNAQYCQMRCTFHPRCLLFSFLPASSINDMEKRFGCFLKDSVTGTLPKVHRTGAVSGHSLKQCGHQISACHRDIYKGVDMRGVNFNVSKVSSVEECQKRCTNNIRCQFFSYATQTFHKAEYRNNCLLKYSPGGTPTAIKVLSNVESGFSLKPCALSEIGCHMNIFQHLAFSDVDVARVLTPDAFVCRTICTYHPNCLFFTFYTNVWKIESQRNVCLLKTSESGTPSSSTPQENTISGYSLLTCKRTLPEPCHSKIYPGVDFGGEELNVTFVKGVNVCQETCTKMIRCQFFTYSLLPEDCKEEKCKCFLRLSMDGSPTRIAYGTQGSSGYSLRLCNTGDNSVCTTKTSTRIVGGTNSSWGEWPWQVSLQVKLTAQRHLCGGSLIGHQWVLTAAHCFDGLPLQDVWRIYSGILNLSDITKDTPFSQIKEIIIHQNYKVSEGNHDIALIKLQAPLNYTEFQKPICLPSKGDTSTIYTNCWVTGWGFSKEKGEIQNILQKVNIPLVTNEECQKRYQDYKITQRMVCAGYKEGGKDACKGDSGGPLVCKHNGMWRLVGITSWGEGCARREQPGVYTKVAEYMDWILEKTQSSDGKAQMQSPA

SEQ ID NO: 4 - protein, 4563 aa, Homo sapiens
MDPPRPALLALLALPALLLLLLAGARAEEEMLENVSLVCPKDATRFKHLRKYTYNYEAESSSGVPGTADSRSATRINCKVELEVPQLCSFILKTSQCTLKEVYGFNPEGKALLKKTKNSEEFAAAMSRYELKLAIPEGKQVFLYPEKDEPTYILNIKRGIISALLVPPETEEAKQVLFLDTVYGNCSTHFTVKTRKGNVATEISTERDLGQCDRFKPIRTGISPLALIKGMTRPLSTLISSSQSCQYTLDAKRKHVAEAICKEQHLFLPFSYKNKYGMVAQVTQTLKLEDTPKINSRFFGEGTKKMGLAFESTKSTSPPKQAEAVLKTLQELKKLTISEQNIQRANLFNKLVTELRGLSDEAVTSLLPQLIEVSSPITLQALVQCGQPQCSTHILQWLKRVHANPLLIDVVTYLVALIPEPSAQQLREIFNMARDQRSRATLYALSHAVNNYHKTNPTGTQELLDIANYLMEQIQDDCTGDEDYTYLILRVIGNMGQTMEQLTPELKSSILKCVQSTKPSLMIQKAAIQALRKMEPKDKDQEVLLQTFLDDASPGDKRLAAYLMLMRSPSQADINKIVQILPWEQNEQVKNFVASHIANILNSEELDIQDLKKLVKEALKESQLPTVMDFRKFSRNYQLYKSVSLPSLDPASAKIEGNLIFDPNNYLPKESMLKTTLTAFGFASADLIEIGLEGKGFEPTLEALFGKQGFFPDSVNKALYWVNGQVPDGVSKVLVDHFGYTKDDKHEQDMVNGIMLSVEKLIKDLKSKEVPEARAYLRILGEELGFASLHDLQLLGKLLLMGARTLQGIPQMIGEVIRKGSKNDFFLHYIFMENAFELPTGAGLQLQISSSGVIAPGAKAGVKLEVANMQAELVAKPSVSVEFVTNMGIIIPDFARSGVQMNTNFFHESGLEAHVALKAGKLKFIIPSPKRPVKLLSGGNTLHLVSTTKTEVIPPLIENRQSWSVCKQVFPGLNYCTSGAYSNASSTDSASYYPLTGDTRLELELRPTGEIEQYSVSATYELQREDRALVDTLKFVTQAEGAKQTEATMTFKYNRQSMTLSSEVQIPDFDVDLGTILRVNDESTEGKTSYRLTLDIQNKKITEVALMGHLSCDTKEERKIKGVISIPRLQAEARSEILAHWSPAKLLLQMDSSATAYGSTVSKRVAWHYDEEKIEFEWNTGTNVDTKKMTSNFPVDLSDYPKSLHMYANRLLDHRVPQTDMTFRHVGSKLIVAMSSWLQKASGSLPYTQTLQDHLNSLKEFNLQNMGLPDFHIPENLFLKSDGRVKYTLNKNSLKIEIPLPFGGKSSRDLKMLETVRTPALHFKSVGFHLPSREFQVPTFTIPKLYQLQVPLLGVLDLSTNVYSNLYNWSASYSGGNTSTDHFSLRARYHMKADSVVDLLSYNVQGSGETTYDHKNTFTLSYDGSLRHKFLDSNIKFSHVEKLGNNPVSKGLLIFDASSSWGPQMSASVHLDSKKKQHLFVKEVKIDGQFRVSSFYAKGTYGLSCQRDPNTGRLNGESNLRFNSSYLQGTNQITGRYEDGTLSLTSTSDLQSGIIKNTASLKYENYELTLKSDTNGKYKNFATSNKMDMTFSKQNALLRSEYQADYESLRFFSLLSGSLNSHGLELNADILGTDKINSGAHKATLRIGQDGISTSATTNLKCSLLVLENELNAELGLSGASMKLTTNGRFREHNAKFSLDGKAALTELSLGSAYQAMILGVDSKNIFNFKVSQEGLKLSNDMMGSYAEMKFDHTNSLNIAGLSLDFSSKLDNIYSSDKFYKQTVNLQLQPYSLVTTLNSDLKYNALDLTNNGKLRLEPLKLHVAGNLKGAYQNNEIKHIYAISSAALSASYKADTVAKVQGVEFSHRLNTDIAGLASAIDMSTNYNSDSLHFSNVFRSVMAPFTMTIDAHTNGNGKLALWGEHTGQLYSKFLLKAEPLAFTFSHDYKGSTSHHLVSRKSISAALEHKVSALLTPAEQTGTWKLKTQFNNNEYSQDLDAYNTKDKIGVELTGRTLADLTLLDSPIKVPLLLSEPINIIDALEMRDAVEKPQEFTIVAFVKYDKNQDVHSINLPFFETLQEYFERNRQTIIVVLENVQRNLKHINIDQFVRKYRAALGKLPQQANDYLNSFNWERQVSHAKEKLTALTKKYRITENDIQIALDDAKINFNEKLSQLQTYMIQFDQYIKDSYDLHDLKIAIANIIDEIIEKLKSLDEHYHIRVNLVKTIHDLHLFIENIDFNKSGSSTASWIQNVDTKYQIRIQIQEKLQQLKRHIQNIDIQHLAGKLKQHIEAIDVRVLLDQLGTTISFERINDILEHVKHFVINLIGDFEVAEKINAFRAKVHELIERYEVDQQIQVLMDKLVELAHQYKLKETIQKLSNVLQQVKIKDYFEKLVGFIDDAVKKLNELSFKTFIEDVNKFLDMLIKKLKSFDYHQFVDETNDKIREVTQRLNGEIQALELPQKAEALKLFLEETKATVAVYLESLQDTKITLIINWLQEALSSASLAHMKAKFRETLEDTRDRMYQMDIQQELQRYLSLVGQVYSTLVTYISDWWTLAAKNLTDFAEQYSIQDWAKRMKALVEQGFTVPEIKTILGTMPAFEVSLQALQKATFQTPDFIVPLTDLRIPSVQINFKDLKNIKIPSRFSTPEFTILNTFHIPSFTIDFVEMKVKIIRTIDQMLNSELQWPVPDIYLRDLKVEDIPLARITLPDFRLPEIAIPEFIIPTLNLNDFQVPDLHIPEFQLPHISHTIEVPTFGKLYSILKIQSPLFTLDANADIGNGTTSANEAGIAASITAKGESKLEVLNFDFQANAQLSNPKINPLALKESVKFSSKYLRTEHGSEMLFFGNAIEGKSNTVASLHTEKNTLELSNGVIVKINNQLTLDSNTKYFHKLNIPKLDFSSQADLRNEIKTLLKAGHIAWTSSGKGSWKWACPRFSDEGTHESQISFTIEGPLTSFGLSNKINSKHLRVNQNLVYESGSLNFSKLEIQSQVDSQHVGHSVLTAKGMALFGEGKAEFTGRHDAHLNGKVIGTLKNSLFFSAQPFEITASTNNEGNLKVRFPLRLTGKIDFLNNYALFLSPSAQQASWQVSARFNQYKYNQNFSAGNNENIMEAHVGINGEANLDFLNIPLTIPEMRLPYTIITTPPLKDFSLWEKTGLKEFLKTTKQSFDLSVKAQYKKNKHRHSITNPLAVLCEFISQSIKSFDRHFEKNRNNALDFVTKSYNETKIKFDKYKAEKSHDELPRTFQIPGYTVPVVNVEVSPFTIEMSAFGYVFPKAVSMPSFSILGSDVRVPSYTLILPSLELPVLHVPRNLKLSLPDFKELCTISHIFIPAMGNITYDFSFKSSVITLNTNAELFNQSDIVAHLLSSSSSVIDALQYKLEGTTRLTRKRGLKLATALSLSNKFVEGSHNSTVSLTTKNMEVSVATTTKAQIPILRMNFKQELNGNTKSKPTVSSSMEFKYDFNSSMLYSTAKGAVDHKLSLESLTSYFSIESSTKGDVKGSVLSREYSGTIASEANTYLNSKSTRSSVKLQGTSKIDDIWNLEVKENFAGEATLQRIYSLWEHSTKNHLQLEGLFFTNGEHTSKATLELSPWQMSALVQVHASQPSSFHDFPDLGQEVALNANTKNQKIRWKNEVRIHSGSFQSQVELSNDQEKAHLDIAGSLEGHLRFLKNIILPVYDKSLWDFLKLDVTTSIGRRQHLRVSTAFVYTKNPNGYSFSIPVKVLADKFIIPGLKLNDLNSVLVMPTFHVPFTDLQVPSCKLDFREIQIYKKLRTSSFALNLPTLPEVKFPEVDVLTKYSQPEDSLIPFFEITVPESQLTVSQFTLPKSVSDGIAALDLNAVANKIADFELPTIIVPEQTIEIPSIKFSVPAGIVIPSFQALTARFEVDSPVYNATWSASLKNKADYVETVLDSTCSSTVQFLEYELNVLGTHKIEDGTLASKTKGTFAHRDFSAEYEEDGKYEGLQEWEGKAHLNIKSPAFTDLHLRYQKDKKGISTSAASPAVGTVGMDMDEDDDFSKWNFYYSPQSSPDKKLTIFKTELRVRESDEETQIKVNWEEEAASGLLTSLKDNVPKATGVLYDYVNKYHWEHTGLTLREVSSKLRRNLQNNAEWVYQGAIRQIDDIDVRFQKAASGTTGTYQEWKDKAQNLYQELLTQEGQASFQGLKDNVFDGLVRVTQEFHMKVKHLIDSLIDFLNFPRFQFPGKPGIYTREELCTMFIREVGTVLSQVYSKVHNGSEILFSYFQDLVITLPFELRKHKLIDVISMYRELLKDLSKEAQEVFKAIQSLKTTEVLRNLQDLLQFIFQLIEDNIKQLKEMKFTYLINYIQDEINTIFSDYIPYVFKLLKENLCLNLHKFNEFIQNELQEASQELQQIHQYIMALREEYFDPSIVGWTVKYYELEEKIVSLIKNLLVALKDFHSEYIVSASNFTSQLSSQVEQFLHRNIQEYLSILTDPDGKGKEKIAELSATAQEIIKSQAIATKKIISDYHQQFRYKLQDFSDQLSDYYEKFIAESKRLIDLSIQNYHTFLIYITELLKKLQSTTVMNPYMKLAPGELTIIL

SEQ ID NO: 5 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 6 - protein, 340 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNSSQDVTVPCRVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTYAVTSILRVAAEDWKKGETFSCMVGHEALPLAFTQKTIDRMAGKPTHINVSVVMAEADGTCY

SEQ ID NO: 7 - protein, 698 aa, Homo sapiens
MRLAVGALLVCAVLGLCLAVPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPEAGYFAIAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 8 - protein, 367 aa, Homo sapiens
MKSLVLLLCLAQLWGCHSAPHGPGLIYRQPNCDDPETEEAALVAIDYINQNLPWGYKHTLNQIDEVKVWPQQPSGELFEIEIDTLETTCHVLDPTPVARCSVRQLKEHAVEGDCDFQLLKLDGKFSVVYAKCDSSPDSAEDVRKVCQDCPLLAPLNDTRVVHAAKAALAAFNAQNNGSNFQLEEISRAQLVPLPPSTYVEFTVSGTDCVAKEATEAAKCNLLAEKQYGFCKATLSEKLGGAEVAVTCMVFQTQPVSSQPQPEGANEAVPTPVVDPDAPPSPPLGAPGLPPAGSPPDSHVLLAAPPGHQLHRAHYDLRHTFMGVVSLGSPSGEVSHPRKTRTVVQPSVGAAAGPVVPPCPGRIRHFKV

SEQ ID NO: 9 - protein, 423 aa, Homo sapiens
MERMLPLLALGLLAAGFCPAVLCHPNSPLDEENLTQENQDRGTHVDLGLASANVDFAFSLYKQLVLKAPDKNVIFSPLSISTALAFLSLGAHNTTLTEILKGLKFNLTETSEAEIHQSFQHLLRTLNQSSDELQLSMGNAMFVKEQLSLLDRFTEDAKRLYGSEAFATDFQDSAAAKKLINDYVKNGTRGKITDLIKDLDSQTMMVLVNYIFFKAKWEMPFDPQDTHQSRFYLSKKKWVMVPMMSLHHLTIPYFRDEELSCTVVELKYTGNASALFILPDQDKMEEVEAMLLPETLKRWRDSLEFREIGELYLPKFSISRDYNLNDILLQLGIEEAFTSKADLSGITGARNLAVSQVVHKAVLDVFEEGTEASAATAVKITLLSALVETRTIVRFNRPFLMIIVPTDTQNIFFMSKVTNPKQA

SEQ ID NO: 10 - protein, 478 aa, Homo sapiens
MAPLRPLLILALLAWVALADQESCKGRCTEGFNVDKKCQCDELCSYYQSCCTDYTAECKPQVTRGDVFTMPEDEYTVYDDGEEKNNATVHEQVGGPSLTSDLQAQSKGNPEQTPVLKPEEEAPAPEVGASKPEGIDSRPETLHPGRPQPPAEEELCSGKPFDAFTDLKNGSLFAFRGQYCYELDEKAVRPGYPKLIRDVWGIEGPIDAAFTRINCQGKTYLFKGSQYWRFEDGVLDPDYPRNISDGFDGIPDNVDAALALPAHSYSGRERVYFFKGKQYWEYQFQHQPSQEECEGSSLSAVFEHFAMMQRDSWEDIFELLFWGRTSAGTRQPQFISRDWHGVPGQVDAAMAGRIYISGMAPRPSLAKKQRFRHRNRKGYRSQRGHSRGRNQNSRRPSRATWLSLFSSEESNLGANNYDDYRMDWLVPATCEPIQSVFFFSGDKYYRVNLRTRRVDTVDPPYPRSIAQYWLGCPAPGHL

SEQ ID NO: 11 - protein, 499 aa, Homo sapiens
MKHSLNALLIFLIITSAWGGSKGPLDQLEKGGETAQSADPQWEQLNNKNLSMPLLPADFHKENTVTNDWIPEGEEDDDYLDLEKIFSEDDDYIDIVDSLSVSPTDSDVSAGNILQLFHGKSRIQRLNILNAKFAFNLYRVLKDQVNTFDNIFIAPVGISTAMGMISLGLKGETHEQVHSILHFKDFVNASSKYEITTIHNLFRKLTHRLFRRNFGYTLRSVNDLYIQKQFPILLDFKTKVREYYFAEAQIADFSDPAFISKTNNHIMKLTKGLIKDALENIDPATQMMILNCIYFKGSWVNKFPVEMTHNHNFRLNEREVVKVSMMQTKGNFLAANDQELDCDILQLEYVGGISMLIVVPHKMSGMKTLEAQLTPRVVERWQKSMTNRTREVLLPKFKLEKNYNLVESLKLMGIRMLFDKNGNMAGISDQRIAIDLFKHQGTITVNEEGTQATTVTTVGFMPLSTQVRFTVDRPFLFLIYEHRTSCLLFMGRVANPSRS

SEQ ID NO: 12 - protein, 453 aa, Homo sapiens
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 13 - protein, 644 aa, Homo sapiens
MKLITILFLCSRLLLSLTQESQSEEIDCNDKDLFKAVDAALKKYNSQNQSNNQFVLYRITEATKTVGSDTFYSFKYEIKEGDCPVQSGKTWQDCEYKDAAKAATGECTATVGKRSSTKFSVATQTCQITPAEGPVVTAQYDCLGCVHPISTQSPDLEPILRHGIQYFNNNTQHSSLFMLNEVKRAQRQVVAGLNFRITYSIVQTNCSKENFLFLTPDCKSLWNGDTGECTDNAYIDIQLRIASFSQNCDIYPGKDFVQPPTKICVGCPRDIPTNSPELEETLTHTITKLNAENNATFYFKIDNVKKARVQVVAGKKYFIDFVARETTCSKESNEELTESCETKKLGQSLDCNAEVYVVPWEKKIYPTVNCQPLGMISLMKRPPGFSPFRSSRIGEIKEETTVSPPHTSMAPAQDEERDSGKEQGHTRRHDWGHEKQRKHNLGHGHKHERDQGHGHQRGHGLGHGHEQQHGLGHGHKFKLDDDLEHQGGHVLDHGHKHKHGHGHGKHKNKGKKNGKHNGWKTEHLASSSEDSTTPSAQTQEKTEGPTPIPSLAKPGVTVTFSDFQDSDLIATMMPPISPAPIQSDDDWIPDIQIDPNGLSFNPISDFPDTTSPKCPGRPWKSVSEINPTTQMKESYYFDLTDGLS

SEQ ID NO: 14 - protein, 19 aa, Homo sapiens
FSEFWDLDPEVRPTSAVAA

SEQ ID NO: 15 - protein, 22 aa, Homo sapiens
GCVLLSYLNETVTVSASLESVR

SEQ ID NO: 16 - protein, 21 aa, Homo sapiens
DKFSEFWDLDPEVRPTSAVAA

SEQ ID NO: 17 - protein, 19 aa, Homo sapiens
VSNQTLSLFFTVLQDVPVR

SEQ ID NO: 18 - protein, 19 aa, Homo sapiens
LQAPLNYTEFQKPICLPSK

SEQ ID NO: 19 - protein, 33 aa, Homo sapiens
QVFPGLNYCTSGAYSNASSTDSASYYPLTGDTR

SEQ ID NO: 20 - protein, 27 aa, Homo sapiens
LSLHRPALEDLLLGSEANLTCTLTGLR

SEQ ID NO: 21 - protein, 13 aa, Homo sapiens
CGLVPVLAENYNK

SEQ ID NO: 22 - protein, 15 aa, Homo sapiens
VCQDCPLLAPLNDTR

SEQ ID NO: 23 - protein, 20 aa, Homo sapiens
FNLTETSEAEIHQSFQHLLR

SEQ ID NO: 24 - protein, 23 aa, Homo sapiens
NNATVHEQVGGPSLTSDLQAQSK

SEQ ID NO: 25 - protein, 13 aa, Homo sapiens
NLSMPLLPADFHK

SEQ ID NO: 26 - protein, 11 aa, Homo sapiens
YKNNSDISSTR

SEQ ID NO: 27 - protein, 12 aa, Homo sapiens
ITYSIVQTNCSK

SEQ ID NO: 28 - protein, 21 aa, Homo sapiens
GLTFQQNASSMCVPDQDTAIR

SEQ ID NO: 29 - protein, 418 aa, Homo sapiens
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 30 - protein, 597 aa, Homo sapiens
MHPPKTPSGALHRKRKMAAWPFSRLWKVSDPILFQMTLIAALLPAVLGNCGPPPTLSFAAPMDITLTETRFKTGTTLKYTCLPGYVRSHSTQTLTCNSDGEWVYNTFCIYKRCRHPGELRNGQVEIKTDLSFGSQIEFSCSEGFFLIGSTTSRCEVQDRGVGWSHPLPQCEIVKCKPPPDIRNGRHSGEENFYAYGFSVTYSCDPRFSLLGHASISCTVENETIGVWRPSPPTCEKITCRKPDVSHGEMVSGFGPIYNYKDTIVFKCQKGFVLRGSSVIHCDADSKWNPSPPACEPNSCINLPDIPHASWETYPRPTKEDVYVVGTVLRYRCHPGYKPTTDEPTTVICQKNLRWTPYQGCEALCCPEPKLNNGEITQHRKSRPANHCVYFYGDEISFSCHETSRFSAICQGDGTWSPRTPSCGDICNFPPKIAHGHYKQSSSYSFFKEEIIYECDKGYILVGQAKLSCSYSHWSAPAPQCKALCRKPELVNGRLSVDKDQYVEPENVTIQCDSGYGVVGPQSITCSGNRTWYPEVPKCEWETPEGCEQVLTGKRLMQCLPNPEDVKMALEVYKLSLEIEQLELQRDSARQSTLDKEL

SEQ ID NO: 31 - protein, 1065 aa, Homo sapiens
MKILILGIFLFLCSTPAWAKEKHYYIGIIETTWDYASDHGEKKLISVDTEHSNIYLQNGPDRIGRLYKKALYLQYTDETFRTTIEKPVWLGFLGPIIKAETGDKVYVHLKNLASRPYTFHSHGITYYKEHEGAIYPDNTTDFQRADDKVYPGEQYTYMLLATEEQSPGEGDGNCVTRIYHSHIDAPKDIASGLIGPLIICKKDSLDKEKEKHIDREFVVMFSVVDENFSWYLEDNIKTYCSEPEKVDKDNEDFQESNRMYSVNGYTFGSLPGLSMCAEDRVKWYLFGMGNEVDVHAAFFHGQALTNKNYRIDTINLFPATLFDAYMVAQNPGEWMLSCQNLNHLKAGLQAFFQVQECNKSSSKDNIRGKHVRHYYIAAEEIIWNYAPSGIDIFTKENLTAPGSDSAVFFEQGTTRIGGSYKKLVYREYTDASFTNRKERGPEEEHLGILGPVIWAEVGDTIRVTFHNKGAYPLSIEPIGVRFNKNNEGTYYSPNYNPQSRSVPPSASHVAPTETFTYEWTVPKEVGPTNADPVCLAKMYYSAVDPTKDIFTGLIGPMKICKKGSLHANGRQKDVDKEFYLFPTVFDENESLLLEDNIRMFTTAPDQVDKEDEDFQESNKMHSMNGFMYGNQPGLTMCKGDSVVWYLFSAGNEADVHGIYFSGNTYLWRGERRDTANLFPQTSLTLHMWPDTEGTFNVECLTTDHYTGGMKQKYTVNQCRRQSEDSTFYLGERTYYIAAVEVEWDYSPQREWEKELHHLQEQNVSNAFLDKGEFYIGSKYKKVVYRQYTDSTFRVPVERKAEEEHLGILGPQLHADVGDKVKIIFKNMATRPYSIHAHGVQTESSTVTPTLPGETLTYVWKIPERSGAGTEDSACIPWAYYSTVDQVKDLYSGLIGPLIVCRRPYLKVFNPRRKLEFALLFLVFDENESWYLDDNIKTYSDHPEKVNKDDEEFIESNKMHAINGRMFGNLQGLTMHVGDEVNWYLMGMGNEIDLHTVHFHGHSFQYKHRGVYSSDVFDIFPGTYQTLEMFPRTPGIWLLHCHVTDHIHAGMETTYTVLQNEDTKSG

SEQ ID NO: 32 - protein, 752 aa, Homo sapiens
MGPLMVLFCLLFLYPGLADSAPSCPQNVNISGGTFTLSHGWAPGSLLTYSCPQGLYPSPASRLCKSSGQWQTPGATRSLSKAVCKPVRCPAPVSFENGIYTPRLGSYPVGGNVSFECEDGFILRGSPVRQCRPNGMWDGETAVCDNGAGHCPNPGISLGAVRTGFRFGHGDKVRYRCSSNLVLTGSSERECQGNGVWSGTEPICRQPYSYDFPEDVAPALGTSFSHMLGATNPTQKTKESLGRKIQIQRSGHLNLYLLLDCSQSVSENDFLIFKESASLMVDRIFSFEINVSVAIITFASEPKVLMSVLNDNSRDMTEVISSLENANYKDHENGTGTNTYAALNSVYLMMNNQMRLLGMETMAWQEIRHAIILLTDGKSNMGGSPKTAVDHIREILNINQKRNDYLDIYAIGVGKLDVDWRELNELGSKKDGERHAFILQDTKALHQVFEHMLDVSKLTDTICGVGNMSANASDQERTPWHVTIKPKSQETCRGALISDQWVLTAAHCFRDGNDHSLWRVNVGDPKSQWGKEFLIEKAVISPGFDVFAKKNQGILEFYGDDIALLKLAQKVKMSTHARPICLPCTMEANLALRRPQGSTCRDHENELLNKQSVPAHFVALNGSKLNINLKMGVEWTSCAEVVSQEKTMFPNLTDVREVVTDQFLCSGTQEDESPCKGESGGAVFLERRFRFFQVGLVSWGLYNPCLGSADKNSRKRAPRSKVPPPRDFHINLFRMQPWLRQHLGDVLNFLPL

SEQ ID NO: 33 - protein, 462 aa, Homo sapiens
MARVLGAPVALGLWSLCWSLAIATPLPPTSAHGNVAEGETKPDPDVTERCSDGWSFDATTLDDNGTMLFFKGEFVWKSHKWDRELISERWKNFPSPVDAAFRQGHNSVFLIKGDKVWVYPPEKKEKGYPKLLQDEFPGIPSPLDAAVECHRGECQAEGVLFFQGDREWFWDLATGTMKERSWPAVGNCSSALRWLGRYYCFQGNQFLRFDPVRGEVPPRYPRDVRDYFMPCPGRGHGHRNGTGHGNSTHHGPEYMRCSPHLVLSALTSDNHGATYAFSGTHYWRLDTSRDGWHSWPIAHQWPQGPSAVDAAFSWEEKLYLVQGTQVYVFLTKGGYTLVSGYPKRLEKEVGTPHGIILDSVDAAFICPGSSRLHIMAGRRLWWLDLKSGAQATWTELPWPHEKVDGALCMEKSLGPNSCSANGPGLYLIHGPNLYCYSDVEKLNAAKALPQPQNVTSLLGCTH

SEQ ID NO: 34 - protein, 525 aa, Homo sapiens
MKALIAALLLITLQYSCAVSPTDCSAVEPEAEKALDLINKRRRDGYLFQLLRIADAHLDRVENTTVYYLVLDVQESDCSVLSRKYWNDCEPPDSRRPSEIVIGQCKVIATRHSHESQDLRVIDFNCTTSSVSSALANTKDSPVLIDFFEDTERYRKQANKALEKYKEENDDFASFRVDRIERVARVRGGEGTGYFVDFSVRNCPRHHFPRHPNVFGFCRADLFYDVEALDLESPKNLVINCEVFDPQEHENINGVPPHLGHPFHWGGHERSSTTKPPFKPHGSRDHHHPHKPHEHGPPPPPDERDHSHGPPLPQGPPPLLPMSCSSCQHATFGTNGAQRHSHNNNSSDLHPHKHHSHEQHPHGHHPHAHHPHEHDTHRQHPHGHHPHGHHPHGHHPHGHHPHGHHPHCHDFQDYGPCDPPPHNQGHCCHGHGPPPGHLRRRGPGKGPRPFHCRQIGSVYRLPPLRKGEVLPLPEANFPSFPLPHHKHPLKPDNQPFPQSVSESCPGKFKSGFPQVSMFFTHTFPK

SEQ ID NO: 35 - protein, 500 aa, Homo sapiens
MASRLTLLTLLLLLLAGDRASSNPNATSSSSQDPESLQDRGEGKVATTVISKMLFVEPILEVSSLPTTNSTTNSATKITANTTDEPTTQPTTEPTTQPTIQPTQPTTQLPTDSPTQPTTGSFCPGPVTLCSDLESHSTEAVLGDALVDFSLKLYHAFSAMKKVETNMAFSPFSIASLLTQVLLGAGENTKTNLESILSYPKDFTCVHQALKGFTTKGVTSVSQIFHSPDLAIRDTFVNASRTLYSSSPRVLSNNSDANLELINTWVAKNTNNKISRLLDSLPSDTRLVLLNAIYLSAKWKTTFDPKKTRMEPFHFKNSVIKVPMMNSKKYPVAHFIDQTLKAKVGQLQLSHNLSLVILVPQNLKHRLEDMEQALSPSVFKAIMEKLEMSKFQPTLLTLPRIKVTTSQDMLSIMEKLEFFDFSYDLNLCGLTEDPDLQVSAMQHQTVLELTETGVEAAAASAISVARTLLVFEVQQPFLFVLWDQQHKFPVFMGRVYDPRA

SEQ ID NO: 36 - protein, 330 aa, Homo sapiens [sequence omitted]

SEQ ID NO: 37 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 38 - protein, 638 aa, Homo sapiens; identical to SEQ ID NO: 3

SEQ ID NO: 39 - protein, 698 aa, Homo sapiens; identical to SEQ ID NO: 7

SEQ ID NO: 40 - protein, 478 aa, Homo sapiens; identical to SEQ ID NO: 10

SEQ ID NO: 41 - protein, 32 aa, Homo sapiens
ADTHDEILEGLNFNLTEIPEAQIHEGFQELLR

SEQ ID NO: 42 - protein, 30 aa, Homo sapiens
FSLLGHASISCTVENETIGVWRPSPPTCEK

SEQ ID NO: 43 - protein, 16 aa, Homo sapiens
EHEGAIYPDNTTDFQR

SEQ ID NO: 44 - protein, 14 aa, Homo sapiens
QSVPAHFVALNGSK

SEQ ID NO: 45 - protein, 13 aa, Homo sapiens
SWPAVGNCSSALR

SEQ ID NO: 46 - protein, 19 aa, Homo sapiens
VIDFNCTTSSVSSALANTK

SEQ ID NO: 47 - protein, 8 aa, Homo sapiens
DTFVNASR

SEQ ID NO: 48 - protein, 19 aa, Homo sapiens
VLSNNSDANLELINTWVAK

SEQ ID NO: 49 - protein, 9 aa, Homo sapiens
EEQYNSTYR

SEQ ID NO: 50 - protein, 9 aa, Homo sapiens
EEQFNSTFR

SEQ ID NO: 51 - protein, 8 aa, Homo sapiens
GVNFNVSK

SEQ ID NO: 52 - protein, 21 aa, Homo sapiens
QQQHLFGSNVTDCSGNFCLFR

SEQ ID NO: 53 - protein, 23 aa, Homo sapiens
NNATVHEQVGGPSLTSDLQAQSK

SEQ ID NO: 54 - protein, 7 aa, synthetic construct
ECDMHYK

SEQ ID NO: 55 - protein, 7 aa, synthetic construct
  SITE 2: note = acetamide-modified Cysteine
ECDMHYK

(19) United States
(12) Patent Application Publication
(10) Pub. No.: US 2025/0232874 A1
(43) Pub. Date: Jul. 17, 2025

(54) AI-DRIVEN GLYCOPROTEOMICS LIQUID BIOPSY IN NASOPHARYNGEAL CARCINOMA

(71) Applicant: VENN BIOSCIENCES CORPORATION, South San Francisco, CA (US)

(72) Inventors: Lee Cheng SIANG, Kuala Lumpur (MY); Daniel SERIE, South San Francisco, CA (US); Karina ISLA RIOS, South San Francisco, CA (US); Thin Thin AYE, South San Francisco, CA (US)

(21) Appl. No.: 18/705,107

(22) PCT Filed: Oct. 28, 2022
(86) PCT No.: PCT/MY2022/050100

§ 371 Date: Apr. 26, 2024

Related U.S. Application Data
(60) Provisional application No. 63/273,864, filed on Oct. 29, 2021.

(51) Int. Cl. G16H 50/20; G16B 15/30; G16B 40/10; G16B 40/20; G16H 20/17

(52) U.S. Cl. CPC G16H 50/20; G16B 15/30; G16B 40/10; G16B 40/20; G16H 20/17

(57) ABSTRACT

A method and system for diagnosing a subject with respect to a nasopharyngeal carcinoma (NPC) disease state. Peptide structure data corresponding to a biological sample obtained from the subject is received. The peptide structure data is analyzed using a supervised machine learning model to generate a disease indicator that indicates whether biological sample evidences the NPC disease state based on at least 3 peptide structures selected from a group of peptide structures identified in Table 1A and/or 1B. The group of peptide structures in Table 1A and/or 1B comprises a group of peptide structures associated with the NPC disease state. The group of peptide structures is listed in Table 1A and/or 1B with respect to relative significance to the disease indicator. A diagnosis output is generated based on the disease indicator.

### CROSS-REFERENCED TO RELATED APPLICATIONS

This application claims priority to U.S. Provisional Patent Application Ser. No. 63/273,864, filed Oct. 29, 2021, which is incorporated by reference herein in its entirety.

### FIELD

The present disclosure generally relates to methods and systems for analyzing peptide structures for diagnosing and/or treating nasopharyngeal carcinoma. More particularly, the present disclosure relates to analyzing quantification data for a set of peptide structures detected in a biological sample obtained from a subject for use in diagnosing and/or treating the subject, the set of peptide structures being associated with nasopharyngeal carcinoma.

### BACKGROUND

Protein glycosylation and other post-translational modifications play vital roles in virtually all aspects of human physiology. Unsurprisingly, faulty or altered protein glycosylation often accompanies various disease states. The identification of aberrant glycosylation provides opportunities for early detection, intervention, and treatment of affected subjects. Current biomarker identification methods, such as those developed in the fields of proteomics and genomics, can be used to detect indicators of certain diseases, such as cancer, and to differentiate certain types of cancer from other, non-cancerous diseases. However, the use of glycoproteomic analyses has not previously been used to successfully identify disease processes.

Glycoprotein analysis is fraught with challenges on several levels. For example, a single glycan composition in a peptide can contain a large number of isomeric structures due to different glycosidic linkages, branching patterns, and/or multiple monosaccharides having the same mass. In addition, the presence of multiple glycans that share the same peptide backbone can lead to assay signals from various glycoforms, lowering their individual abundances compared to aglycosylated peptides. Accordingly, the development of algorithms that can identify glycan structures on peptide fragments remains elusive.

In light of the above, there is a need for improved analytical methods that involve site-specific analysis of glycoproteins to obtain information about protein glycosylation patterns, which can in turn provide quantitative information that can be used to identify disease states. For example, there is a need to use such analysis to diagnose and/or treat nasopharyngeal carcinoma (NPC).

Diagnosing and treating NPC currently relies on physical examination followed by examination of a blood sample for Epstein Bar Virus (EBV). Physical examination can involve a medical practitioner checking for lumps on the neck, lips, gums, and cheeks of a patient. As with many physical examinations, results are often inconclusive and require additional testing. In the case of NPC, blood can be examined for the presence of EBV antibodies. A challenge with this approach includes a high rate of false positives and low accuracy results. A contributing factor includes EBV antibodies being present in the absence of NPC which indicates NPC and EBV may not be related in all cases.

An approach that is both non-invasive and includes a low false positive rate while maintaining a high level of accuracy is needed. Protein glycosylation and other post-translational modifications play vital roles in virtually all aspects of human physiology. Unsurprisingly, faulty or altered protein glycosylation often accompanies disease. The identification of aberrant glycosylation provides opportunities for early detection, intervention, and treatment of affected subjects.

In light of the above, there is a desire for improved analytical methods that involve site-specific analysis of glycoproteins to obtain information about protein glycosylation patterns as they relate to NPC, which can in turn provide quantitative information that can be used to manage the treatment of a subject. Thus, it may be desirable to have methods and systems capable of addressing one or more of the above-identified issues.

### SUMMARY

In one or more embodiments, a method for diagnosing a subject with respect to a nasopharyngeal carcinoma (NPC) disease state is provided. Peptide structure data corresponding to a biological sample obtained from the subject is received. The peptide structure data is analyzed using a supervised machine learning model to generate a disease indicator that indicates whether biological sample evidences the NPC disease state based on at least 3 peptide structures selected from a group of peptide structures identified in Table 1A and/or 1B. The group of peptide structures in Table 1A and/or 1B is associated with the NPC disease state. The group of peptide structures is listed in Table 1A and/or 1B with respect to relative significance to the disease indicator. A diagnosis output is generated based on the disease indicator.

In one or more embodiments, a method is provided for training a model to diagnose a subject with respect to a nasopharyngeal carcinoma (NPC) disease state. Quantification data is received for a panel of peptide structures for a plurality of subjects. The plurality of subjects includes a first portion diagnosed with the NPC disease state and a second portion diagnosed with a non-NPC state. The quantification data comprises a plurality of peptide structure profiles for the plurality of subjects. A machine learning model is trained, using the quantification data, to diagnose a biological sample with respect to the NPC disease state using a group of peptide structures associated with the NPC disease state. The group of peptide structures is identified in Table 1A and/or 1B. The group of peptide structures is listed in Table 1A and/or 1B with respect to relative significance to making the diagnosis.

In one or more embodiments, a method is provided for treating nasopharyngeal carcinoma (NPC) in a patient. A biological sample is received from a patient. A quantity of each peptide structure identified in Table 1A and/or 1B in the biological sample is determined using MRM-MS. The quantity of each peptide structure is analyzed using a machine learning model to generate a disease indicator. A diagnosis output is generated based on the disease indicator that classifies the biological sample as evidencing an NPC disease state. A treatment is administered to the patient based on at least one of the diagnosis output or the disease indicator.

In one or more embodiments, a method is provided for monitoring a subject for a nasopharyngeal carcinoma (NPC). Peptide structure data of a first biological sample obtained from a subject at a first timepoint is received. The peptide structure data of the first biological sample is analyzed using a supervised machine learning model to generate a first disease indicator based on at least 3 peptide structures selected from a group of peptide structures identified in Table 1A and/or 1B. The group of peptide structures in Table 1A and/or 1B comprises a group of peptide structures associated with the NPC disease state. Peptide structure data of a second biological sample obtained from the subject at a second timepoint is received. The peptide structure data of the second biological sample is analyzed using the supervised machine learning model to generate a second disease indicator based on the at least 3 peptide structures selected from the group of peptide structures identified in Table 1A and/or 1B. A diagnosis output is generated based on the first disease indicator and the second disease indicator.

In one or more embodiments, a composition comprises a peptide structure or a product ion. The peptide structure or product ion comprises an amino acid sequence having at least 90% sequence identity to any one of SEQ ID NOS: 14-28, corresponding to peptide structures PS-1 to PS-19 in Table 1A and/or the peptide structure or product ion comprises an amino acid sequence having at least 90% sequence identity to any one of SEQ ID NOS: 15, 20, or 41-53 of Table 1B. The product ion is selected as one from a group consisting of product ions identified in Table 4A or 4B including product ions falling within an identified m/z range.

In one or more embodiments, a composition comprises a glycopeptide structure selected as one from a group consisting of peptide structures PS-1 to PS-19 identified in Table 1A and/or selected as one from a group consisting of peptide structures PS-20 to PS-35 identified in Table 1B. The glycopeptide structure comprises: an amino acid peptide sequence identified in Table 5A or 5B as corresponding to the glycopeptide structure; and a glycan structure identified in Table 7A or 7B as corresponding to the glycopeptide structure in which the glycan structure is linked to a residue of the amino acid peptide sequence at a corresponding position identified in Table 1A or 1B, respectively. In another embodiment, the glycopeptide structure comprises: an amino acid peptide sequence identified in Table 5A or 5B as corresponding to the glycopeptide structure; and a glycan composition identified in Table 7A or 7B as corresponding to the glycopeptide structure in which the glycan composition is linked to a residue of the amino acid peptide sequence at a corresponding position identified in Table 1A or 1B, respectively.

In one or more embodiments, a composition comprises a peptide structure selected as one from a plurality of peptide structures identified in Table 1A and/or 1B. The peptide structure has a monoisotopic mass identified as corresponding to the peptide structure in Table 1. The peptide structure comprises the amino acid sequence of SEQ ID NOs: 14-28 identified in Table 1A as corresponding to the peptide structure and/or the peptide structure comprises the amino acid sequence of SEQ ID NOs: 15, 20, or 41-53 identified in Table 1B as corresponding to the peptide structure.

In one or more embodiments, a kit comprises at least one agent for quantifying at least one peptide structure identified in Table 1A and/or 1B to carry out part or all of any one or more of the methods described herein.

In one or more embodiments, a kit comprises at least one of a glycopeptide standard, a buffer, or a set of peptide sequences to carry out part or all of any one or more of the methods described herein, a peptide sequence of the set of peptide sequences identified by a corresponding one of SEQ ID NOS: 14-28, defined in Table 1A and/or a peptide sequence of the set of peptide sequences identified by a corresponding one of SEQ ID NOS: 15, 20, or 41-53, defined in Table 1B.

In one or more embodiments, a system comprises one or more data processors; and a non-transitory computer readable storage medium containing instructions which, when executed on the one or more data processors, cause the one or more data processors to perform part or all of any one or more of the methods described herein.

In one or more embodiments, a computer-program product tangibly embodied in a non-transitory machine-readable storage medium is provided that includes instructions configured to cause one or more data processors to perform part or all of any one or more of the methods described herein.

### BRIEF DESCRIPTION OF THE DRAWINGS

The present disclosure is described in conjunction with the appended figures:

FIG. 1 is a schematic diagram of an exemplary workflow 100 for the detection of peptide structures associated with a disease state for use in diagnosis and/or treatment in accordance with one or more embodiments.

FIG. 2A is a schematic diagram of a preparation workflow in accordance with one or more embodiments. FIG. 2A discloses SEQ ID NOS 54-55, respectively, in order of appearance.

FIG. 2B is a schematic diagram of data acquisition in accordance with one or more embodiments.

FIG. 3 is a block diagram of an analysis system in accordance with one or more embodiments.

FIG. 4 is a block diagram of a computer system in accordance with various embodiments.

FIG. 5 is a flowchart of a process for diagnosing a subject with respect to a nasopharyngeal carcinoma (NPC) disease state in accordance with one or more embodiments.

FIG. 6 is a flowchart of a process for training a model to diagnose a subject with respect to a nasopharyngeal carcinoma (NPC) disease state in accordance with one or more embodiments.

FIG. 7 is a flowchart of a process for monitoring a subject for a nasopharyngeal carcinoma (NPC) in accordance with one or more embodiments.

FIG. 8 is a flowchart of a process for treating a subject for HCC in accordance with one or more embodiments.

FIG. 9 is a table of the sample population used for the experiments in accordance with one or more embodiments.

FIG. 10 is an illustration of a volcano plot highlighting differentially expressed peptides between NPC cases and control in accordance with one or embodiments.

FIG. 11 is an illustration of a heat map depicting the 62 peptide structures that significantly differentially expressed between the healthy/control and NPC cancer patients in accordance with one or embodiments.

FIG. 12A is a plot diagram illustrating validation of the disease indicator's ability to determine whether a biological sample evidences the NPC disease state or a non-NPC state (e.g., healthy, control, etc.) in accordance with one or embodiments.

FIG. 12B is a plot diagram showing the probability distributions for the various groups using the multivariable model for predicting the NPC state vs. Healthy controls in accordance with one or more embodiments.

FIG. 12C is a plot diagram of the probability distributions for healthy, hospital, stage 1 NPC, stage 2 NPC, stage 3 NPC, stage 4 NPC, and suspected samples.

FIG. 13A is a receiver operating characteristic (ROC) curve in accordance with one or embodiments.

FIG. 13B is ROC curve in accordance with one or embodiments.

FIG. 14A shows the model performance metrics of accuracy, sensitivity, positive predictive value (PPV), negative predictive value (NPV), and harmonic mean of precision and sensitivity (F1) for the training data set and test set, respectively.

FIG. 14B shows the model performance metrics of accuracy by stage for the training data set and test set, respectively.

### DETAILED DESCRIPTION

### I. Overview

The embodiments described herein recognize that glycoproteomics is an emerging field that can be used in the overall diagnosis and/or treatment of subjects with various types of diseases. Glycoproteomics aims to determine the positions, identities, and quantities of glycans and glycosylated proteins in a given sample (e.g., blood sample, cell, tissue, etc.). Protein glycosylation is one of the most common and most complex forms of post-translational protein modification, and can affect protein structure, conformation, and function. For example, glycoproteins may play crucial roles in important biological processes such as cell signaling, host-pathogen interactions, and immune response and disease. Glycoproteins may therefore be important to diagnosing different types of diseases.

Although protein glycosylation provides useful information about cancer and other diseases, analysis of protein glycosylation may be difficult as the glycan typically cannot be traced back to the protein site of origin with currently available methodologies. Glycoprotein analysis can be challenging in general due to several reasons. For example, a single glycan composition in a peptide may contain a large number of isomeric structures because of different glycosidic linkages, branching, and many monosaccharides having the same mass. Further, the presence of multiple glycans that share the same peptide sequence may cause the mass spectrometry (MS) signal to split into various glycoforms, lowering their individual abundances compared to the peptides that are not glycosylated (aglycosylated peptides).

But to understand various disease conditions and to more accurately diagnose certain diseases, such as nasopharyngeal carcinoma (NPC), it may be important to perform analysis of glycoproteins and to identify not only the glycan but also the linking site (e.g., the amino acid residue of attachment) within the protein. Thus, there is a need to provide a method for site-specific glycoprotein analysis to obtain detailed information about protein glycosylation patterns which may be able to provide information about a disease state (e.g., a nasopharyngeal carcinoma (NPD) disease state). This information can be used to distinguish the disease state from other states, diagnose a subject as having or not having the disease state, determine a likelihood that a subject has the disease state, or a combination thereof. For example, such analysis may be useful in diagnosing whether a subject has an NPC state or not (e.g., is healthy or part of a control population).

Accordingly, the embodiments described herein provide various methods and systems for analyzing proteins in subjects and, in particular, glycoproteins. In one or more embodiments, a machine learning model is trained to analyze peptide structure data and generate a disease indicator that provides information relating to one or more diseases. For example, in various embodiments, the peptide structure data comprises quantification metrics (e.g., abundance or concentration data) for peptide structures. A peptide structure may be defined by an aglycosylated peptide sequence (e.g., a peptide or peptide fragment of a larger parent protein) or a glycosylated peptide sequence. A glycosylated peptide sequence (also referred to as a glycopeptide structure) may be a peptide sequence having a glycan structure that is attached to a linking site (e.g., an amino acid residue) of the peptide sequence, which may occur via, for example, a particular atom of the amino acid residue). Non-limiting examples of glycosylated peptides include N-linked glycopeptides and O-linked glycopeptides.

The embodiments described herein recognize that the abundance of selected peptide structures in a biological sample obtained from a subject may be used to determine the likelihood of that subject having an NPC disease state. An NPC disease state may include any condition that can be diagnosed as cancer that occurs in the nasopharynx. Further, certain peptide structures that are associated with an NPC disease state may be more relevant to that disease state than other peptide structures that are also associated with that disease state.

Analyzing the abundance of peptide sequences and glycosylated peptide sequences in a biological sample may provide a more accurate way in which to distinguish the NPC disease state from a non-NPC disease state (e.g., healthy state, control state, etc.) This type of peptide structure analysis may be more conducive to generating accurate diagnoses as compared to glycoprotein analysis that focuses on analyzing glycoproteins that are too large to be resolved via mass spectrometry. Further, with glycoproteins, there may be too many potential proteoforms to consider. Still further, analysis of peptide structure data in the manner described by the various embodiments herein may be more conducive to generating accurate diagnoses as compared to glycomic analysis that provides little to no information about what proteins and to which amino acid residue sites various glycan structures attach.

The description below provides exemplary implementations of the methods and systems described herein for the research, diagnosis, and/or treatment of an NPC disease state. Descriptions and examples of various terms, as used herein, are provided in Section II below.

### II. Exemplary Descriptions of Terms

The term “ones” means more than one.

As used herein, the term “plurality” may be 2, 3, 4, 5, 6, 7, 8, 9, 10, or more.

As used herein, the term “set of” means one or more. For example, a set of items includes one or more items.

As used herein, the phrase “at least one of,” when used with a list of items, means different combinations of one or more of the listed items may be used and only one of the items in the list may be needed. The item may be a particular object, thing, step, operation, process, or category. In other words, “at least one of” means any combination of items or number of items may be used from the list, but not all of the items in the list may be required. For example, without limitation, “at least one of item A, item B, or item C” means item A; item A and item B; item B; item A, item B, and item C; item B and item C; or item A and C. In some cases, “at least one of item A, item B, or item C” means, but is not limited to, two of item A, one of item B, and ten of item C; four of item B and seven of item C; or some other suitable combination.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

As used herein, “abundance,” may refer to a quantitative value generated using mass spectrometry. In various embodiments, the quantitative value may relate to an amount of a particular peptide structure (e.g., biomarker) present in a biological sample. In some embodiments, the amount may be in relation to other structures present in the sample (e.g., relative abundance) In some embodiments, the quantitative value may comprise an amount of an ion produced using mass spectrometry.

The term “amino acid,” as used herein, generally refers to any organic compound that includes an amino group (e.g., —NH2), a carboxyl group (—COOH), and a side chain group (R) which varies based on a specific amino acid. Amino acids can be linked using peptide bonds.

The term “alkylation,” as used herein, generally refers to the transfer of an alkyl group from one molecule to another. In various embodiments, alkylation is used to react with reduced cysteines to prevent the re-formation of disulfide bonds after reduction has been performed.

The term “linking site” or “glycosylation site” as used herein generally refers to the location where a sugar molecule of a glycan or glycan structure is directly bound (e.g., covalently bound) to an amino acid of a peptide, a polypeptide, or a protein. For example, the linking site may be an amino acid residue and a glycan structure may be linked via an atom of the amino acid residue. Non-limiting examples of types of glycosylation can include N-linked glycosylation, O-linked glycosylation, C-linked glycosylation, S-linked glycosylation, and glycation.

The terms “biological sample,” “biological specimen,” or “biospecimen” as used herein, generally refers to a specimen taken by sampling so as to be representative of the source of the specimen, typically, from a subject. A biological sample can be representative of an organism as a whole, specific tissue, cell type, or category or sub-category of interest. The biological sample can include a macromolecule. The biological sample can include a small molecule. The biological sample can include a virus. The biological sample can include a cell or derivative of a cell. The biological sample can include an organelle. The biological sample can include a cell nucleus. The biological sample can include a rare cell from a population of cells. The biological sample can include any type of cell, including without limitation prokaryotic cells, eukaryotic cells, bacterial, fungal, plant, mammalian, or other animal cell type, mycoplasmas, normal tissue cells, tumor cells, or any other cell type, whether derived from single cell or multicellular organisms. The biological sample can include a constituent of a cell. The biological sample can include nucleotides (e.g., ssDNA, dsDNA, RNA), organelles, amino acids, peptides, proteins, carbohydrates, glycoproteins, or any combination thereof. The biological sample can include a matrix (e.g., a gel or polymer matrix) comprising a cell or one or more constituents from a cell (e.g., cell bead), such as DNA, RNA, organelles, proteins, or any combination thereof, from the cell. The biological sample may be obtained from a tissue of a subject. The biological sample can include a hardened cell. Such hardened cells may or may not include a cell wall or cell membrane. The biological sample can include one or more constituents of a cell but may not include other constituents of the cell. An example of such constituents may include a nucleus or an organelle. The biological sample may include a live cell. The live cell can be capable of being cultured.

The term “biomarker,” as used herein, generally refers to any measurable substance taken as a sample from a subject whose presence is indicative of some phenomenon. Non-limiting examples of such phenomenon can include a disease state, a condition, or exposure to a compound or environmental condition. In various embodiments described herein, biomarkers may be used for diagnostic purposes (e.g., to diagnose a health state, a disease state). The term “biomarker” can be used interchangeably with the term “marker.”

The term “denaturation,” as used herein, generally refers to any molecule that loses quaternary structure, tertiary structure, and secondary structure which is present in their native state. Non-limiting examples include proteins or nucleic acids being exposed to an external compound or environmental condition such as acid, base, temperature, pressure, radiation, etc.

The term “denatured protein,” as used herein, generally refers to a protein that loses quaternary structure, tertiary structure, and secondary structure which is present in their native state.

The terms “digestion” or “enzymatic digestion,” as used herein, generally refer to breaking apart a polymer (e.g., cutting a polypeptide at a cut site). Proteins may be digested in preparation for mass spectrometry using trypsin digestion protocols. Proteins may be digested using other proteases in preparation for mass spectrometry if access is limited to cleavage sites.

The terms “immune checkpoint inhibitor therapeutic” and “immune checkpoint inhibitor drug,” as used herein, generally refer to drugs or therapeutics that can target immune checkpoint molecules (e.g., molecules on immune cells that need to be activated (or inactivated) to start an immune response). Non-limiting examples of immune checkpoint inhibitor therapeutics can include pembrolizumab, nivolumab, and cemiplimab.

The term “disease state” as used herein, generally refers to a condition that affects the structure or function of an organism. Non-limiting examples of causes of disease states may include pathogens, immune system dysfunctions, cell damage caused by aging, cell damage caused by other factors (e.g., trauma and cancer). Disease states can include any state of a disease whether symptomatic or asymptomatic. Disease states can include disease stages of a disease progression. Disease states can cause minor, moderate, or severe disruptions in structure or function of an organism (e.g., a subject).

The terms “glycan” or “polysaccharide” as used herein, both generally refer to a carbohydrate residue of a glycoconjugate, such as the carbohydrate portion of a glycopeptide, glycoprotein, glycolipid, or proteoglycan. Glycans can include monosaccharides.

The term “glycopeptide” or “glycopolypeptide” as used herein, generally refer to a peptide or polypeptide comprising at least one glycan residue. In various embodiments, glycopeptides comprise carbohydrate moieties (e.g., one or more glycans) covalently attached to a side chain (i.e. R group) of an amino acid residue.

The term “glycoprotein,” as used herein, generally refers to a protein having at least one glycan residue bonded thereto. In some examples, a glycoprotein is a protein with at least one oligosaccharide chain covalently bonded thereto. Examples of glycoproteins, include but are not limited to apolipoprotein C-III (APOC3), alpha-1-antichymotrypsin (AACT), afamin (AFAM), alpha-1-acid glycoprotein 1 & 2 (AGP12), apolipoprotein B-100 (APOB), apolipoprotein D (APOD), complement C1s subcomponent (CIS), calpain-3 (CAN3), clusterin (CLUS), complement component C8AChain (CO8A), alpha-2-HS-glycoprotein (FETUA), haptoglobin (HPT), immunoglobulin heavy constant gamma 1 (IgG1), immunoglobulin J chain (IgJ), plasma kallikrein (KLKB1), serum paraoxonase/arylesterase 1 (PON1), prothrombin (THRB), serotransferrin (TRFE), protein unc-13 homologA (UN13A), zinc-alpha-2-glycoprotein (ZA2G), Alpha-1-antitrypsin (A1AT), Alpha-2-macroglobulin (A2MG), C4b-binding protein alpha chain (C4BPA), Ceruloplasmin (CERU), Complement C2 (CO2), Hemopexin (HEMO), Histidine-rich glycoprotein (HRG), Plasma protease C1 inhibitor (IC1), Immunoglobulin heavy constant alpha 1 (IGA1), Immunoglobulin heavy constant alpha 2 (IGA2), Immunoglobulin heavy constant gamma 1 (IGG1), Immunoglobulin heavy constant gamma 2 (IGG2), Serotransferrin (TRFE), prothrombin (THRB), serotransferrin (TRFE), and Vitronectin (VTNC). A glycopeptide, as used herein, refers to a fragment of a glycoprotein, unless specified otherwise to the contrary.

The term “liquid chromatography,” as used herein, generally refers to a technique used to separate a sample into parts. Liquid chromatography can be used to separate, identify, and quantify components.

The term “mass spectrometry,” as used herein, generally refers to an analytical technique used to identify molecules. In various embodiments described herein, mass spectrometry can be involved in characterization and sequencing of proteins.

The term “m/z” or “mass-to-charge ratio” as used herein, generally refers to an output value from a mass spectrometry instrument. In various embodiments, m/z can represent a relationship between the mass of a given ion and the number of elementary charges that it carries. The “m” in m/z stands for mass and the “z” standards for charge number of ions. In some embodiments, m/z can be displayed on an x-axis of a mass spectrum.

The term “peptide,” as used herein, generally refers to amino acids linked by peptide bonds. Peptides can include amino acid chains between 10 and 50 residues. Peptides can include amino acid chains shorter than 10 residues, including, oligopeptides, dipeptides, tripeptides, and tetrapeptides. Peptides can include chains longer than 50 residues and may be referred to as “polypeptides” or “proteins.”

The terms “protein” or “polypeptide” or “peptide” may be used interchangeably herein and generally refer to a molecule including at least three amino acid residues. Proteins can include polymer chains made of amino acid sequences linked together by peptide bonds. Proteins may be digested in preparation for mass spectrometry using trypsin digestion protocols. Proteins may be digested using other proteases in preparation for mass spectrometry if access is limited to cleavage sites.

The term “peptide structure,” as used herein, generally refers to peptides or a portion thereof or glycopeptides or a portion thereof. In various embodiments described herein, a peptide structure can include any molecule comprising at least two amino acids in sequence.

The term “reduction,” as used herein, generally refers to the gain of an electron by a substance. In various embodiments described herein, a sugar can directly bind to a protein, thereby, reducing the amino acid to which it binds. Such reducing reactions can occur in glycosylation. In various embodiments, reduction may be used to break disulfide bonds between two cysteines.

The term “sample,” as used herein, generally refers to a sample from a subject of interest and may include a biological sample of a subject. The sample may include a cell sample. The sample may include a cell line or cell culture sample. The sample can include one or more cells. The sample can include one or more microbes. The sample may include a nucleic acid sample or protein sample. The sample may also include a carbohydrate sample or a lipid sample. The sample may be derived from another sample. The sample may include a tissue sample, such as a biopsy, core biopsy, needle aspirate, or fine needle aspirate. The sample may include a fluid sample, such as a blood sample, urine sample, or saliva sample. The sample may include a skin sample. The sample may include a cheek swab. The sample may include a plasma or serum sample. The sample may include a cell-free or cell free sample. A cell-free sample may include extracellular polynucleotides. The sample may originate from blood, plasma, serum, urine, saliva, mucosal excretions, sputum, stool, or tears. The sample may originate from red blood cells or white blood cells. The sample may originate from feces, spinal fluid, CNS fluid, gastric fluid, amniotic fluid, cyst fluid, peritoneal fluid, marrow, bile, other body fluids, tissue obtained from a biopsy, skin, or hair.

The term “sequence,” as used herein, generally refers to a biological sequence including one-dimensional monomers that can be assembled to generate a polymer. Non-limiting examples of sequences include nucleotide sequences (e.g., ssDNA, dsDNA, and RNA), amino acid sequences (e.g., proteins, peptides, and polypeptides), and carbohydrates (e.g., compounds including Cₘ (H₂O)ₙ).

The term “subject,” as used herein, generally refers to an animal, such as a mammal (e.g., human) or avian (e.g., bird), or other organism, such as a plant. For example, the subject can include a vertebrate, a mammal, a rodent (e.g., a mouse), a primate, a simian or a human. Animals may include, but are not limited to, farm animals, sport animals, and pets. A subject can include a healthy or asymptomatic individual, an individual that has or is suspected of having a disease (e.g., cancer) or a pre-disposition to the disease, and/or an individual that is in need of therapy or suspected of needing therapy. A subject can be a patient. A subject can include a microorganism or microbe (e.g., bacteria, fungi, archaea, viruses).

The term “training data,” as used herein generally refers to data that can be input into models, statistical models, algorithms and any system or process able to use existing data to make predictions.

As used herein, a “model” may include one or more algorithms, one or more mathematical techniques, one or more machine learning algorithms, or a combination thereof.

As used herein, “machine learning” may be the practice of using algorithms to parse data, learn from it, and then make a determination or prediction about something in the world. Machine learning uses algorithms that can learn from data without relying on rules-based programming. A machine learning algorithm may include a parametric model, a nonparametric model, a deep learning model, a neural network, a linear discriminant analysis model, a quadratic discriminant analysis model, a support vector machine, a random forest algorithm, a nearest neighbor algorithm, a combined discriminant analysis model, a k-means clustering algorithm, a supervised model, an unsupervised model, logistic regression model, a multivariable regression model, a penalized multivariable regression model, or another type of model.

As used herein, an “artificial neural network” or “neural network” (NN) may refer to mathematical algorithms or computational models that mimic an interconnected group of artificial nodes or neurons that processes information based on a connectionistic approach to computation. Neural networks, which may also be referred to as neural nets, can employ one or more layers of nonlinear units to predict an output for a received input. Some neural networks include one or more hidden layers in addition to an output layer. The output of each hidden layer is used as input to the next layer in the network, i.e., the next hidden layer or the output layer. Each layer of the network generates an output from a received input in accordance with current values of a respective set of parameters. In the various embodiments, a reference to a “neural network” may be a reference to one or more neural networks.

A neural network may process information in two ways: when it is being trained it is in training mode and when it puts what it has learned into practice it is in inference (or prediction) mode. Neural networks learn through a feedback process (e.g., backpropagation) which allows the network to adjust the weight factors (modifying its behavior) of the individual nodes in the intermediate hidden layers so that the output matches the outputs of the training data. In other words, a neural network learns by being fed training data (learning examples) and eventually learns how to reach the correct output, even when it is presented with a new range or set of inputs. A neural network may include, for example, without limitation, at least one of a Feedforward Neural Network (FNN), a Recurrent Neural Network (RNN), a Modular Neural Network (MNN), a Convolutional Neural Network (CNN), a Residual Neural Network (ResNet), an Ordinary Differential Equations Neural Networks (neural-ODE), or another type of neural network.

As used herein, a “target glycopeptide analyte,” may refer to a peptide structure (e.g., glycosylated or aglycosylated/non-glycosylated), a fraction of a peptide structure, a sub-structure (e.g., a glycan or a glycosylation site) of a peptide structure, a product of one or more of the above listed structures and sub-structures, associated detection molecules (e.g., signal molecule, label, or tag), or an amino acid sequence that can be measured by mass spectrometry.

As used herein, a “a peptide data set,” may be used interchangeably with “peptide structure data” and can refer to any data of or relating to a peptide from a resulting mass spectrometry run such as, for example, quantitation data. A peptide data set can comprise data obtained from a sample or biological sample using mass spectrometry. A peptide dataset can comprise data relating to a NGEP external standard, data relating to an internal standard, and data relating to a target glycopeptide analyte of a sample. A peptide data set can result from analysis originating from a single run. In some embodiments, the peptide data set can include raw abundance and mass to charge ratios for one or more peptides.

As used herein, a “non-glycosylated endogenous peptide” (“NGEP”), may refer to a peptide structure that does not comprise a glycan molecule. In various embodiments, an NGEP and a target glycopeptide analyte can originate from the same subject. In various embodiments, an NGEP can be labeled with an isotope in preparation for mass spectrometry analysis.

As used herein, a “a transition,” may refer to or identify a peptide structure. In some embodiments, a transition can refer to the specific pair of m/z values associated with a precursor ion and a product or fragment ion.

As used herein, a “non-glycosylated endogenous peptide” (“NGEP”) may refer to a peptide structure that does not comprise a glycan molecule. In various embodiments, an NGEP and a target glycopeptide analyte may be derived from the same protein sequence. In some embodiments, the NGEP and the target glycopeptide analyte may be derived from or include the same peptide sequence. In various embodiments, a NGEP can be labeled with an isotope in preparation for mass spectrometry analysis.

As used herein, an “abundance value” may refer to “abundance” or a quantitative value associated with abundance.

As used herein, “relative abundance,” may refer to a comparison of two or more abundances. In various embodiments, the comparison may comprise comparing one peptide structure to a total number of peptide structures. In some embodiments, the comparison may comprise comparing one peptide glycoform (e.g., two identical peptides differing by one or more glycans) to a set of peptide glycoforms. In some embodiments, the comparison may comprise comparing a number of ions having a particular m/z ratio by a total number of ions detected. In various embodiments, a relative abundance can be expressed as a ratio. In other embodiments, a relative abundance can be expressed as a percentage. Relative abundance can be presented on a y-axis of a mass spectrum plot.

As used herein, an “internal standard,” may refer to something that can be contained (e.g., spiked-in) in the same sample as a target glycopeptide analyte undergoing mass spectrometry analysis. Internal standards can be used for calibration purposes. Additionally, internal standards can be used in the systems and method described herein. In some aspects, an internal standard can be selected based on similarity m/z and or retention times and can be a “surrogate” if a specific standard is too costly or unavailable. Internal standards can be heavy labeled or non-heavy labeled.

### III. Overview of Exemplary Workflow

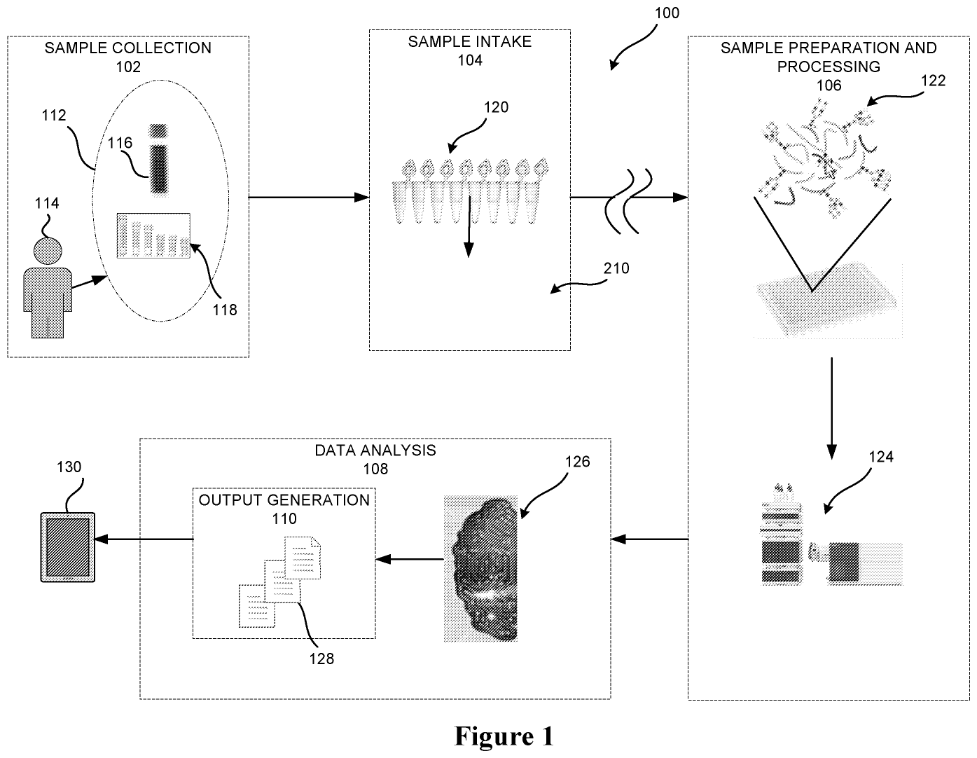

FIG. 1 is a schematic diagram of an exemplary workflow 100 for the detection of peptide structures associated with a disease state for use in diagnosis and/or treatment in accordance with one or more embodiments. Workflow 100 may include various operations including, for example, sample collection 102, sample intake 104, sample preparation and processing 106, data analysis 108, and output generation 110.

Sample collection 102 may include, for example, obtaining a biological sample 112 of one or more subjects, such as subject 114. Biological sample 112 may take the form of a specimen obtained via one or more sampling methods. Biological sample 112 may be representative of subject 114 as a whole or of a specific tissue, cell type, or other category or sub-category of interest. Biological sample 112 may be obtained in any of a number of different ways. In various embodiments, biological sample 112 includes whole blood sample 116 obtained via a blood draw. In other embodiments, biological sample 112 includes set of aliquoted samples 118 that includes, for example, a serum sample, a plasma sample, a blood cell (e.g., white blood cell (WBC), red blood cell (RBC) sample, another type of sample, or a combination thereof. Biological samples 112 may include nucleotides (e.g., ssDNA, dsDNA, RNA), organelles, amino acids, peptides, proteins, carbohydrates, glycoproteins, or any combination thereof.

In various embodiments, a single run can analyze a sample (e.g., the sample including a peptide analyte), an external standard (e.g., an NGEP of a serum sample), and an internal standard. As such, abundance values (e.g., abundance or raw abundance) for the external standard, the internal standard, and target glycopeptide analyte can be determined by mass spectrometry in the same run.

In various embodiments, external standards may be analyzed prior to analyzing samples. In various embodiments, the external standards can be run independently between the samples. In some embodiments, external standards can be analyzed after every 1, 2, 3, 4, 5, 6, 7, 8, 9, 10, 11, 12, 13, 14, 15, 16, 17, 18, 19, 20, or more experiments. In various embodiments, external standard data can be used in some or all of the normalization systems and methods described herein. In additional embodiments, blank samples may be processed to prevent column fouling.

Sample intake 104 may include one or more various operations such as, for example, aliquoting, registering, processing, storing, thawing, and/or other types of operations. In one or more embodiments, when biological sample 112 includes whole blood sample 116, sample intake 104 includes aliquoting whole blood sample 116 to form a set of aliquoted samples that can then be sub-aliquoted to form set of samples 120.

Sample preparation and processing 106 may include, for example, one or more operations to form set of peptide structures 122. In various embodiments, set of peptide structures 122 may include various fragments of unfolded proteins that have undergone digestion and may be ready for analysis.

Further, sample preparation and processing 106 may include, for example, data acquisition 124 based on set of peptide structures 122. For example, data acquisition 124 may include use of, for example, but is not limited to, a liquid chromatography/mass spectrometry (LC/MS) system.

Data analysis 108 may include, for example, peptide structure analysis 126. In some embodiments, data analysis 108 also includes output generation 110. In other embodiments, output generation 110 may be considered a separate operation from data analysis 108. Output generation 110 may include, for example, generating final output 128 based on the results of peptide structure analysis 126. Final output 128 may be used for determining research, diagnosis, and/or treatment.

In various embodiments, final output 128 is comprised of one or more outputs. Final output 128 may take various forms. For example, final output 128 may be a report that includes, for example, a diagnosis output, a treatment output (e.g., a treatment design output, a treatment plan output, or combination thereof), analyzed data (e.g., relativized and normalized) or combination thereof. In some embodiments, report can comprise a target glycopeptide analyte concentration as a function of the NGEP concentration value and the normalized abundance value. In some embodiments, final output 128 may be an alert (e.g., a visual alert, an audible alert, etc.), a notification (e.g., a visual notification, an audible notification, an email notification, etc.), an email output, or a combination thereof. In some embodiments, final output 128 may be sent to remote system 130 for processing. Remote system 130 may include, for example, a computer system, a server, a processor, a cloud computing platform, cloud storage, a laptop, a tablet, a smartphone, some other type of mobile computing device, or a combination thereof.

In other embodiments, workflow 100 may optionally exclude one or more of the operations described herein and/or may optionally include one or more other steps or operations other than those described herein (e.g., in addition to and/or instead of those described herein). Accordingly, workflow 100 may be implemented in any of a number of different ways for use in the research, diagnosis, and/or treatment of a disease state.

### IV. Detection and Quantification of Peptide Structures

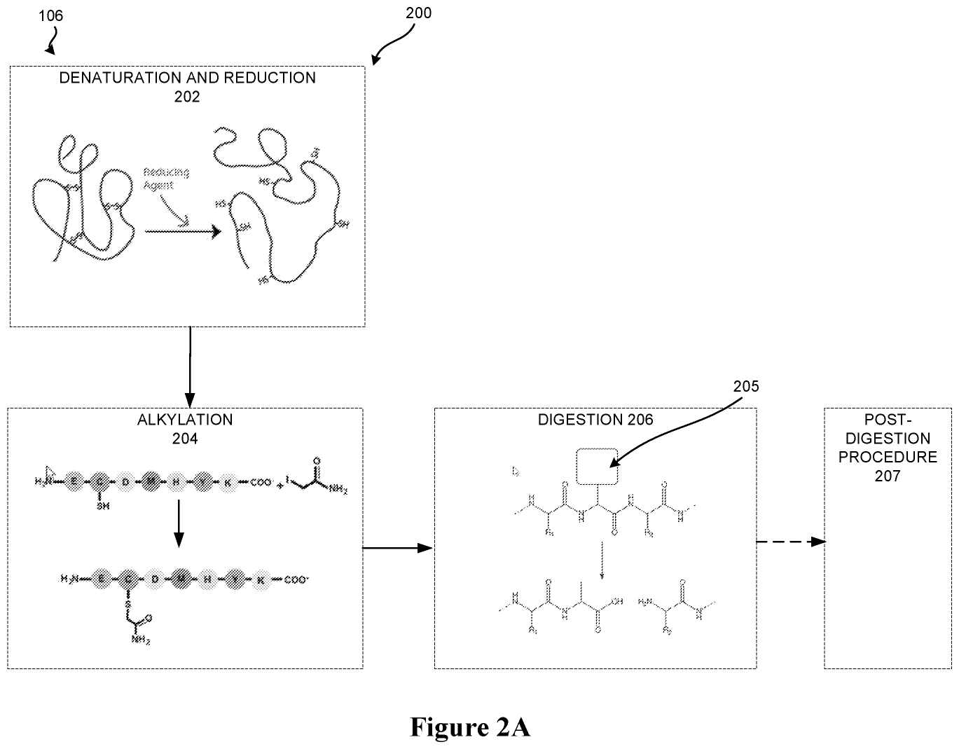

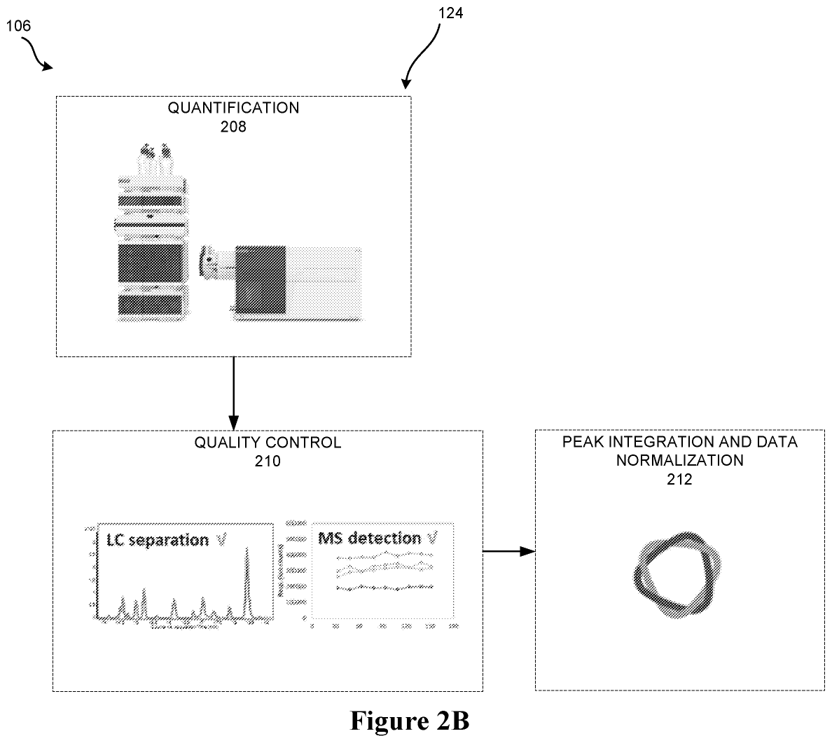

FIGS. 2A and 2B are schematic diagrams of a workflow for sample preparation and processing 106 in accordance with one or more embodiments. FIGS. 2A and 2B are described with continuing reference to FIG. 1. Sample preparation and processing 106 may include, for example, preparation workflow 200 shown in FIG. 2A and data acquisition 124 shown in FIG. 2B.

### IV.A. Sample Preparation and Processing

FIG. 2A is a schematic diagram of preparation workflow 200 in accordance with one or more embodiments. Preparation workflow 200 may be used to prepare a sample, such as a sample of set of samples 120 in FIG. 1, for analysis via data acquisition 124. For example, this analysis may be performed via mass spectrometry (e.g., LC-MS). In various embodiments, preparation workflow 200 may include denaturation and reduction 202, alkylation 204, and digestion 206. All areas of the preparation workflow can cause inconsistency between different samples and different experiments, necessitating, the improved normalization systems and methods described herein and throughout.

In general, polymers, such as proteins, in their native form, can fold to include secondary, tertiary, and/or other higher order structures. Such higher order structures may functionalize proteins to complete tasks (e.g., enable enzymatic activity) in a subject. Further, such higher order structures of polymers may be maintained via various interactions between side chains of amino acids within the polymers. Such interactions can include ionic bonding, hydrophobic interactions, hydrogen bonding, and disulfide linkages between cysteine residues. However, when using analytic systems and methods, including mass spectrometry, unfolding such polymers (e.g., peptide/protein molecules) may be desired to obtain sequence information. In some embodiments, unfolding a polymer may include denaturing the polymer, which may include, for example, linearizing the polymer.

In one or more embodiments, denaturation and reduction 202 can be used to disrupt higher order structures (e.g., secondary, tertiary, quaternary, etc.) of one or more proteins (e.g., polypeptides and peptides) in a sample (e.g., one of set of samples 120 in FIG. 1). Denaturation and reduction 202 includes, for example, a denaturation procedure and a reduction procedure. In some embodiments, the denaturation procedure may be performed using, for example, thermal denaturation, where heat is used as a denaturing agent. The thermal denaturation can disrupt ionic bonding, hydrophobic interactions, and/or hydrogen bonding.

In various embodiments, the denaturation procedure may include using one or more denaturing agents. In one or more embodiments, the denaturation procedure may include using temperature. In one or more embodiments, the denaturation procedure may include using one or more denaturing agents in combination with heat. These one or more denaturing agents may include, for example, but are not limited to, any number of chaotropic salts (e.g., urea, guanidine), surfactants (e.g., sodium dodecyl sulfate (SDS), beta octyl glucoside, Triton X-100), or combination thereof. In some cases, such denaturing agents may be used in combination with heat when sample preparation workflow further includes a cleanup procedure.

The resulting one or more denatured (e.g., unfolded, linearized) proteins may then undergo further processing in preparation of analysis. For example, a reduction procedure may be performed in which one or more reducing agents are applied. In various embodiments, a reducing agent can produce an alkaline pH. A reducing agent may take the form of, for example, without limitation, dithiothreitol (DTT), tris(2-carboxyethyl)phosphine (TCEP), or some other reducing agent. The reducing agent may reduce (e.g., cleave) the disulfide linkages between cysteine residues of the one or more denatured proteins to form one or more reduced proteins.

In various embodiments, the one or more reduced proteins resulting from denaturation and reduction 202 may undergo a process to prevent the reformation of disulfide linkages between, for example, the cysteine residues of the one or more reduced proteins. This process may be implemented using alkylation 204 to form one or more alkylated proteins. For example, alkylation 204 may be used to add an acetamide group to a sulfur on each cysteine residue to prevent disulfide linkages from reforming. In various embodiments, an acetamide group can be added by reacting one or more alkylating agents with a reduced protein. The one or more alkylating agents may include, for example, one or more acetamide salts. An alkylating agent may take the form of, for example, iodoacetamide (IAA), 2-chloroacetamide, some other type of acetamide salt, or some other type of alkylating agent.

In some embodiments, alkylation 204 may include a quenching procedure. The quenching procedure may be performed using one or more reducing agents (e.g., one or more of the reducing agents described above).

In various embodiments, the one or more alkylated proteins formed via alkylation 204 can then undergo digestion 206 in preparation for analysis (e.g., mass spectrometry analysis). Digestion 206 of a protein may include cleaving the protein at or around one or more cleavage sites (e.g., site 205 which may be one or more amino acid residues). For example, without limitation, an alkylated protein may be cleaved at the carboxyl side of the lysine or arginine residues. This type of cleavage may break the protein into various segments, which include one or more peptide structures (e.g., glycosylated or aglycosylated).

In various embodiments, digestion 206 is performed using one or more proteolysis catalysts. For example, an enzyme can be used in digestion 206. In some embodiments, the enzyme takes the form of trypsin. In other embodiments, one or more other types of enzymes (e.g., proteases) may be used in addition to or in place of trypsin. These one or more other enzymes include, but are not limited to, LysC, LysN, AspN, GluC, and ArgC. In some embodiments, digestion 206 may be performed using tosyl phenylalanyl chloromethyl ketone (TPCK)-treated trypsin, one or more engineered forms of trypsin, one or more other formulations of trypsin, or a combination thereof. In some embodiments, digestion 206 may be performed in multiple steps, with each involving the use of one or more digestion agents. For example, a secondary digestion, tertiary digestion, etc. may be performed. In one or more embodiments, trypsin is used to digest serum samples. In one or more embodiments, trypsin/LysC cocktails are used to digest plasma samples.

In some embodiments, digestion 206 further includes a quenching procedure. The quenching procedure may be performed by acidifying the sample (e.g., to a pH<3). In some embodiments, formic acid may be used to perform this acidification.

In various embodiments, preparation workflow 200 further includes post-digestion procedure 207. Post-digestion procedure 207 may include, for example, a cleanup procedure. The cleanup procedure may include, for example, the removal of unwanted components in the sample that results from digestion 206. For example, unwanted components may include, but are not limited to, inorganic ions, surfactants, etc. In some embodiments, post-digestion procedure 207 further includes a procedure for the addition of heavy-labeled peptide internal standards.

Although preparation workflow 200 has been described with respect to a sample created or taken from biological sample 112 that is blood-based (e.g., a whole blood sample, a plasma sample, a serum sample, etc.), sample preparation workflow 200 may be similarly implemented for other types of samples (e.g., tears, urine, tissue, interstitial fluids, sputum, etc.) to produce set of peptides structures 122.

### IV.B. Peptide Structure Identification and Quantitation

FIG. 2B is a schematic diagram of data acquisition 124 in accordance with one or more embodiments. In various embodiments, data acquisition 124 can commence following sample preparation 200 described in FIG. 2A. In various embodiments, data acquisition 124 can comprise quantification 208, quality control 210, and peak integration and normalization 212.

In various embodiments, targeted quantification 208 of peptides and glycopeptides can incorporate use of liquid chromatography-mass spectrometry LC/MS instrumentation. For example, LC-MS/MS, or tandem MS may be used. In general, LC/MS (e.g., LC-MS/MS) can combine the physical separation capabilities of liquid chromatograph (LC) with the mass analysis capabilities of mass spectrometry (MS). According to some embodiments described herein, this technique allows for the separation of digested peptides to be fed from the LC column into the MS ion source through an interface.

In various embodiments, any LC/MS device can be incorporated into the workflow described herein. In various embodiments, an instrument or instrument system suited for identification and targeted quantification 208 may include, for example, a Triple Quadrupole LC/MS™. In various embodiments, targeted quantification 208 is performed using multiple reaction monitoring mass spectrometry (MRM-MS).

In various embodiments described herein, identification of a particular protein or peptide and an associated quantity can be assessed. In various embodiments described herein, identification of a particular glycan and an associated quantity can be assessed. In various embodiments described herein, particular glycans can be matched to a glycosylation site on a protein or peptide and the abundance values measured.

In some cases, targeted quantification 208 includes using a specific collision energy associated for the appropriate fragmentation to consistently see an abundant product ion. Glycopeptide structures may have a lower collision energy than aglycosylated peptide structures. When analyzing a sample that includes glycopeptide structures, the source voltage and gas temperature may be lowered as compared to generic proteomic analysis.

In various embodiments, quality control 210 procedures can be put in place to optimize data quality. In various embodiments, measures can be put in place allowing only errors within acceptable ranges outside of an expected value. In various embodiments, employing statistical models (e.g., using Westgard rules) can assist in quality control 210. For example, quality control 210 may include, for example, assessing the retention time and abundance of representative peptide structures (e.g., glycosylated and/or aglycosylated) and spiked-in internal standards, in either every sample, or in each quality control sample (e.g., pooled serum digest).

Peak integration and normalization 212 may be performed to process the data that has been generated and transform the data into a format for analysis. For example, peak integration and normalization 212 may include converting abundance data for various product ions that were detected for a selected peptide structure into a single quantification metric (e.g., a relative quantity, an adjusted quantity, a normalized quantity, a relative concentration, an adjusted concentration, a normalized concentration, etc.) for that peptide structure. In some embodiments, peak integration and normalization 212 may be performed using one or more of the techniques described in U.S. Patent Publication No. 2020/0372973A1 and/or US Patent Publication No. 2020/0240996A1, the disclosures of which are incorporated by reference herein in their entireties.

### V. Peptide Structure Data Analysis

### V.A. Exemplary System for Peptide Structure Data Analysis

### V.A.1. Analysis System for Peptide Structure Data Analysis

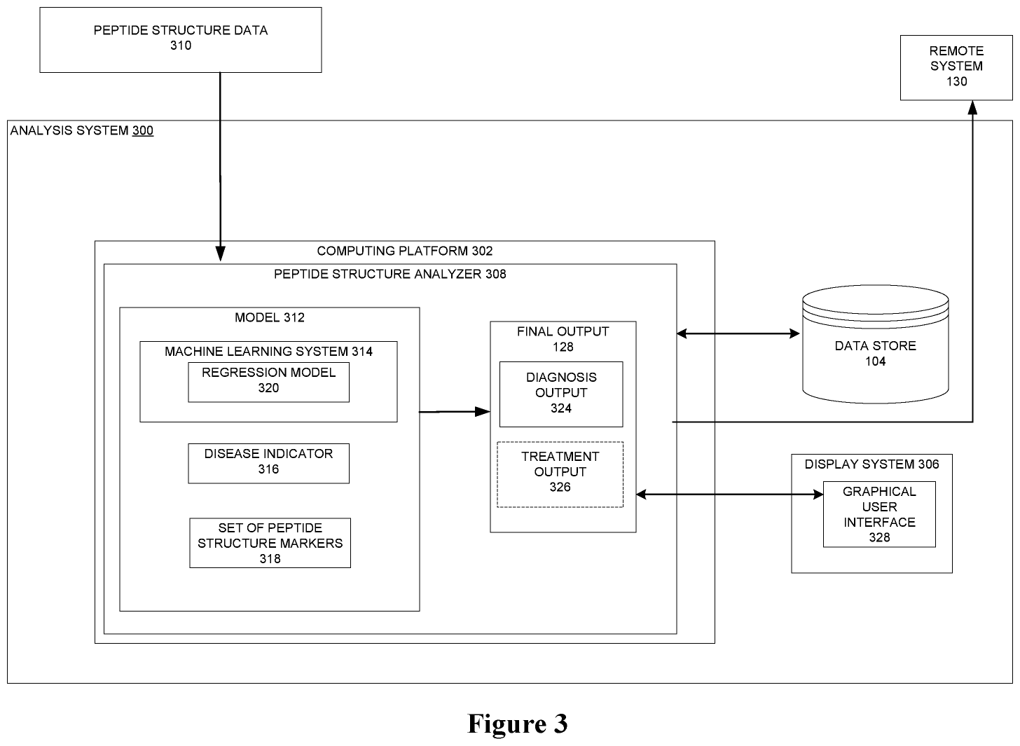

FIG. 3 is a block diagram of an analysis system 300 in accordance with one or more embodiments. Analysis system 300 can be used to both detect and analyze various peptide structures that have been associated to various disease states. Analysis system 300 is one example of an implementation for a system that may be used to perform data analysis 108 in FIG. 1. Thus, analysis system 300 is described with continuing reference to workflow 100 as described in FIGS. 1, 2A, and/or 2B.

Analysis system 300 may include computing platform 302 and data store 304. In some embodiments, analysis system 300 also includes display system 306. Computing platform 302 may take various forms. In one or more embodiments, computing platform 302 includes a single computer (or computer system) or multiple computers in communication with each other. In other examples, computing platform 302 takes the form of a cloud computing platform.

Data store 304 and display system 306 may each be in communication with computing platform 302. In some examples, data store 304, display system 306, or both may be considered part of or otherwise integrated with computing platform 302. Thus, in some examples, computing platform 302, data store 304, and display system 306 may be separate components in communication with each other, but in other examples, some combination of these components may be integrated together. Communication between these different components may be implemented using any number of wired communications links, wireless communications links, optical communications links, or a combination thereof.

Analysis system 300 includes, for example, peptide structure analyzer 308, which may be implemented using hardware, software, firmware, or a combination thereof. In one or more embodiments, peptide structure analyzer 308 is implemented using computing platform 302.

Peptide structure analyzer 308 receives peptide structure data 310 for processing. Peptide structure data 310 may be, for example, the peptide structure data that is output from sample preparation and processing 106 in FIGS. 1, 2A, and 2B. Accordingly, peptide structure data 310 may correspond to set of peptide structures 122 identified for biological sample 112 and may thereby correspond to biological sample 112.

Peptide structure data 310 can be sent as input into peptide structure analyzer 308, retrieved from data store 304 or some other type of storage (e.g., cloud storage), accessed from cloud storage, or obtained in some other manner. In some cases, peptide structure data 310 may be retrieved from data store 304 in response to (e.g., directly or indirectly based on) receiving user input entered by a user via an input device.

Peptide structure analyzer 308 includes model 312 that is configured to receive peptide structure data 310 for processing. Model 312 may be implemented in any of a number of different ways. Model 312 may be implemented using any number of models, functions, equations, algorithms, and/or other mathematical techniques.

In one or more embodiments, model 312 includes machine learning system 314, which may itself be comprised of any number of machine learning models and/or algorithms. For example, machine learning system 314 may include, but is not limited to, at least one of a deep learning model, a neural network, a linear discriminant analysis model, a quadratic discriminant analysis model, a support vector machine, a random forest algorithm, a nearest neighbor algorithm (e.g., a k-Nearest Neighbors algorithm), a combined discriminant analysis model, a k-means clustering algorithm, an unsupervised model, a multivariable regression model, a penalized multivariable regression model, or another type of model. In various embodiments, model 312 includes a machine learning model 314 that comprises any number of or combination of the models or algorithms described above.

In various embodiments, model 312 analyzes peptide structure data 310 to generate disease indicator 316 that indicates whether the biological sample is positive for a nasopharyngeal carcinoma (NPC) disease state based on set of peptide structures 318 identified as being associated with the NPC disease state. Peptide structure data 310 may include quantification data for the plurality of peptide structures. For example, peptide structure data 310 may include a set of quantification metrics for each peptide structure of a plurality of peptide structures. A quantification metric for a peptide structure may be selected as one of a relative quantity, an adjusted quantity, a normalized quantity, a relative abundance, an adjusted abundance, and a normalized abundance. In some cases, a quantification metric for a peptide structure is selected from one of a relative concentration, an adjusted concentration, and a normalized concentration. In this manner, peptide structure data 310 may provide abundance information about the plurality of peptide structures with respect to biological sample 112.

Disease indicator 316 may take various forms. In some cases, disease indicator 316 includes a probability that the subject is positive for the NPC disease state. In other examples, disease indicator 316 includes a classification that indicates whether or not the subject is positive for the NPC disease state.

In some embodiments, a peptide structure of set of peptide structures 318 comprises a glycosylated peptide structure, or glycopeptide structure, that is defined by a peptide sequence and a glycan structure attached to a linking site of the peptide sequence quantity. For example, the peptide structure may be a glycopeptide or a portion of a glycopeptide. In some embodiments, a peptide structure of set of peptide structures 318 comprises an aglycosylated peptide structure that is defined by a peptide sequence. For example, the peptide structure may be a peptide or a portion of a peptide and may be referred to as a quantification peptide.

Set of peptide structures 318 may be identified as being those most predictive or relevant to the NPC disease state based on training of model 312. In one or more embodiments, set of peptide structures 318 includes at least one, at least three, or at least five of the peptide structures identified in Table 1 below in Section VI.A The number of peptide structures selected from Table 1 for inclusion in set of peptide structures 318 may be based on, for example, a desired level of accuracy.

In various embodiments, machine learning model 314 takes the form of regression model 320. Regression model 320 may be, for example, a penalized multivariable regression model that is trained to identify set of peptide structures 318 from a plurality of (or panel of) peptide structures identified in various subjects. Regression model 320 may be trained to identify weight coefficients for peptide structures and those peptide structures having non-zero weights or weight coefficients above a selected threshold (e.g., absolute weight coefficient above 0.1, 0.2, 0.3, etc.) may be selected for inclusion in set of peptide structures 318.

Peptide structure analyzer 308 may generate final output 128 based on disease indicator 316 output by model 312. In other embodiments, final output 128 may be an output generated by model 312.

In some embodiments, final output 128 includes disease indicator 316. In other embodiments, final output 128 includes diagnosis output 324 and/or treatment output 326. Diagnosis output 324 may include, for example, a determination of whether the subject is positive for the NPC disease state based on disease indicator 316. Treatment output 326 may include, for example, at least one of an identification of a therapeutic to treat the subject, a design for the therapeutic, or a treatment plan for administering the therapeutic. In some embodiments, the therapeutic is a targeted drug therapy such as, for example, Cetuximab. In various embodiments, treatment output 326 includes a dosage for the therapeutic to be used in treating the subject. This dosage may be computed based on, for example, the disease indicator. In some embodiments, treatment output 326 may identify one or more different types of treatment including, but not limited to, radiation therapy, chemoradiation therapy, surgery, targeted drug therapy, or a combination thereof.

Final output 128 may be sent to remote system 130 for processing in some examples. In other embodiments, final output 128 may be displayed on graphical user interface 328 in display system 306 for viewing by a human operator. The human operator may use final output 128 to diagnose and/or treat subject when final output 128 indicates the subject is positive for the NPC disease state.

### V.A.2. Computer Implemented System

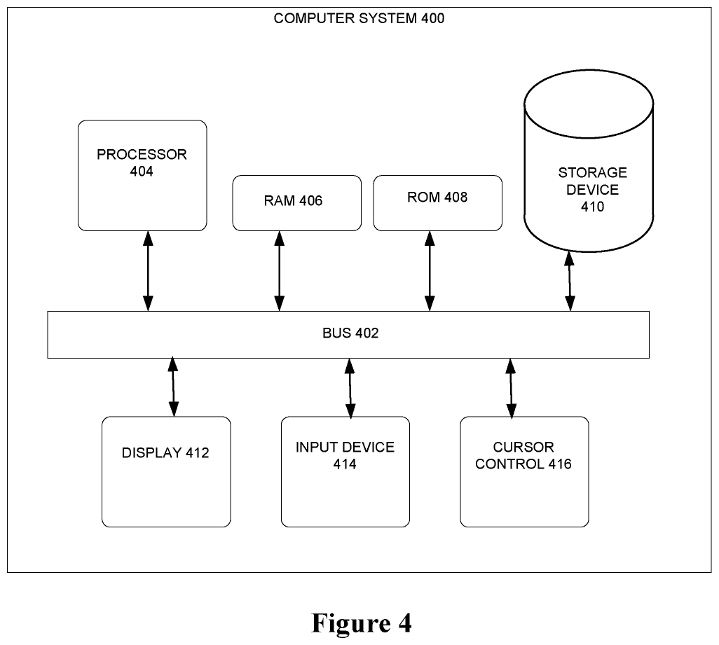

FIG. 4 is a block diagram of a computer system in accordance with various embodiments. Computer system 400 may be an example of one implementation for computing platform 302 described above in FIG. 3.

In one or more examples, computer system 400 can include a bus 402 or other communication mechanism for communicating information, and a processor 404 coupled with bus 402 for processing information. In various embodiments, computer system 400 can also include a memory, which can be a random-access memory (RAM) 406 or other dynamic storage device, coupled to bus 402 for determining instructions to be executed by processor 404. Memory also can be used for storing temporary variables or other intermediate information during execution of instructions to be executed by processor 404. In various embodiments, computer system 400 can further include a read only memory (ROM) 408 or other static storage device coupled to bus 402 for storing static information and instructions for processor 404. A storage device 410, such as a magnetic disk or optical disk, can be provided and coupled to bus 402 for storing information and instructions.

In various embodiments, computer system 400 can be coupled via bus 402 to a display 412, such as a cathode ray tube (CRT) or liquid crystal display (LCD), for displaying information to a computer user. An input device 414, including alphanumeric and other keys, can be coupled to bus 402 for communicating information and command selections to processor 404. Another type of user input device is a cursor control 416, such as a mouse, a joystick, a trackball, a gesture input device, a gaze-based input device, or cursor direction keys for communicating direction information and command selections to processor 404 and for controlling cursor movement on display 412. This input device 414 typically has two degrees of freedom in two axes, a first axis (e.g., x) and a second axis (e.g., y), that allows the device to specify positions in a plane. However, it should be understood that input devices 414 allowing for three-dimensional (e.g., x, y, and z) cursor movement are also contemplated herein.

Consistent with certain implementations of the present teachings, results can be provided by computer system 400 in response to processor 404 executing one or more sequences of one or more instructions contained in RAM 406. Such instructions can be read into RAM 406 from another computer-readable medium or computer-readable storage medium, such as storage device 410. Execution of the sequences of instructions contained in RAM 406 can cause processor 404 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, storage device, data storage device, etc.) or “computer-readable storage medium” as used herein refers to any media that participates in providing instructions to processor 404 for execution. Such a medium can take many forms, including but not limited to, non-volatile media, volatile media, and transmission media. Examples of non-volatile media can include, but are not limited to, optical, solid state, magnetic disks, such as storage device 410. Examples of volatile media can include, but are not limited to, dynamic memory, such as RAM 406. Examples of transmission media can include, but are not limited to, coaxial cables, copper wire, and fiber optics, including the wires that comprise bus 402.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 404 of computer system 400 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, optical communications connections, etc.

It should be appreciated that the methodologies described herein, flow charts, diagrams, and accompanying disclosure can be implemented using computer system 400 as a standalone device or on a distributed network of shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 400, whereby processor 404 would execute the analyses and determinations provided by these engines, subject to instructions provided by any one of, or a combination of, the memory components RAM 406, ROM, 408, or storage device 410 and user input provided via input device 414.

### VI. Exemplary Methodologies Relating to Diagnosis Based on Peptide Structure Data Analysis

### VI.A. General Methodology

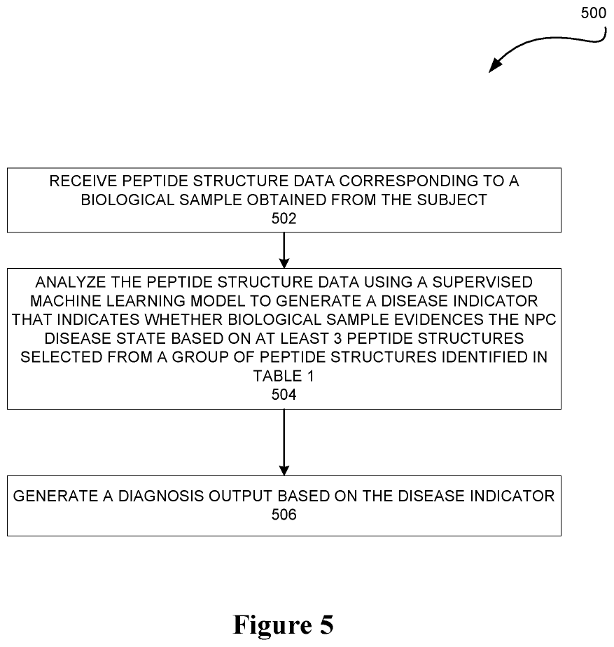

FIG. 5 is a flowchart of a process for diagnosing a subject with respect to a nasopharyngeal carcinoma (NPC) disease state in accordance with one or more embodiments. Process 500 may be implemented using, for example, at least a portion of workflow 100 as described in FIGS. 1, 2A, and 2B and/or analysis system 300 as described in FIG. 3. Process 500 may be used to generate a final output that includes at least a diagnosis output for the subject.

Step 502 includes receiving peptide structure data corresponding to a biological sample obtained from the subject. The peptide structure data may be, for example, one example of an implementation of peptide structure data 310 in FIG. 3. The peptide structure data may include quantification data for each peptide structure of a plurality of peptide structures. The quantification data may include, for example, one or more quantification metrics for each peptide structure of the plurality of peptide structures. A quantification metric for a peptide structure may be, for example, but is not limited to, a relative quantity, an adjusted quantity, a normalized quantity, a relative concentration, an adjusted concentration, or a normalized concentration. In this manner, the quantification data for a given peptide structure provides an indication of the abundance of the peptide structure in the biological sample.

Step 504 includes analyzing the peptide structure data using a supervised machine learning model to generate a disease indicator that indicates whether biological sample evidences the NPC disease state based on at least 3 peptide structures selected from a group of peptide structures identified in Table 1 (below). In step 504, the group of peptide structures is associated with the NPC disease state. The group of peptide structures is listed in Table 1 with respect to relative significance to the disease indicator. At least two peptide structures in the at least 3 peptide structures includes a glycopeptide structure defined by a peptide sequence and a glycan structure linked to a linking site of the peptide sequence.

The disease indicator that is generated may include, for example, at least one of a probability that the subject is positive for the NPC disease state, an odds that the subject is positive for the NPC disease state, a logarithm of the odds that the subject is positive for the NPC disease state, or a classification of the biological sample as either positive or negative for the NPC disease state.

In one or more embodiments, step 504 may be implemented using a regression model. In some examples, the regression model may be, for example, penalized multivariable regression model. In various embodiments, the disease indicator may be computed using a weight coefficient associated with each peptide structure of the at least 3 peptide structures, the weight coefficient of a corresponding peptide structure of the at least 3 peptide structures may indicate the relative significance of the corresponding peptide structure to the disease indicator.

In some embodiments, step 504 may include computing a peptide structure profile for the biological sample that identifies a weighted value for each peptide structure of the at least 3 peptide structures. The weighted value for a peptide structure of the at least 3 peptide structures may be a product of a quantification metric for the peptide structure identified from the peptide structure data and a weight coefficient for the peptide structure. The disease indicator may be computed using the peptide structure profile. For example, the disease indicator may be a logit equal to the sum of the weighted values for the peptide structures plus an intercept value. The intercept value may be determined during the training of the model.

In various embodiments, the disease indicator comprises a probability that the biological sample is positive for the NPC disease state and the supervised machine learning model is configured to generate an output that identifies the biological sample as either evidencing (“positive for”) the NPC disease state when the disease indicator is greater than a selected threshold or not evidencing (“negative for”) the NPC disease state when the disease indicator is not greater than the selected threshold. The selected threshold may be, for example, 0.30, 0.35, 0.40, 0.45, 0.50, 0.55, or some other threshold. In one or more embodiments, the selected threshold is 0.35.

Step 506 includes generating a diagnosis output based on the disease indicator. The diagnosis output may include the disease indicator, or a diagnosis made based on the disease indicator. The diagnosis may be, for example, “positive” if the biological sample evidences the NPC disease state based on the disease indicator or “negative” if the biological sample does not evidence the NPC disease state based on the disease indicator. A negative diagnosis may mean that the biological sample has a non-NPC state (e.g., healthy, control, etc.). The method described in FIG. 5 using Table 1 is applicable to either Table 1A and/or 1B.

TABLE 1A
Peptide Structures associated with NPC

| | | | | Mono- | Linking | Linking | |
|---|---|---|---|---|---|---|---|
| | | (Protein) | (Peptide) | isotopic | Site Pos. in | Site Pos. in | Glycan |
| PS-ID | Peptide Structure | SEQ ID | SEQ ID | mass | Protein | Peptide | Structure |
| NO. | (PS) NAME | NO. | NO. | (Da) | Sequence | Sequence | GL NO. |
| PS-1 | APOC3_74_1300 | 1 | 14 | 2907.31 | 94 | 14 | 1300 |
| PS-2 | APOC3_74_1102 | 1 | 14 | 3083.34 | 94 | 14 | 1102 |
| PS-3 | A2MG_55_5411 | 2 | 15 | 4455.96 | 55 | 9 | 5411 |
| PS-4 | APOC3_74MC_1102 | 1 | 16 | 3326.46 | 94 | 16 | 1102 |
| PS-5 | A2MG_1424_5402_Z3 | 2 | 17 | 4366.95 | 1424 | 3 | 5402 |
| PS-6 | KLKB1_494_5400 | 3 | 18 | 3868.76 | 494 | 6 | 5400 |
| PS-7 | APOB_983_5401 | 4 | 19 | 5463.24 | 983 | 16 | 5401 |
| PS-8 | IGA1_144_3500 | 5 | 20 | 4464.15 | 144 | 18 | 3500 |
| PS-9 | IGA2_131_3500 | 6 | 20 | 4464.15 | 144 | 18 | 3500 |
| PS-10 | TRFE_432_5402 | 7 | 21 | 3680.52 | 432 | 12 | 5402 |
| PS-11 | FETUA_156_5400 | 8 | 22 | 3393.42 | 156 | 12 | 5400 |
| PS-12 | KLKB1_494_6503 | 3 | 18 | 5107.18 | 494 | 6 | 6503 |
| PS-13 | A2MG_1424_5412 | 2 | 17 | 4513.00 | 1424 | 3 | 5412 |
| PS-14 | AACT_106_7604 | 9 | 23 | 5916.41 | 106 | 2 | 7604 |
| PS-15 | VTNC_86_6503 | 10 | 24 | 5241.16 | 86 | 2 | 6503 |
| PS-16 | HEP2_49_5412 | 11 | 25 | 3832.60 | 49 | 1 | 5412 |
| PS-17 | IGM_46_4310 | 12 | 26 | 2687.12 | 46 | 3 | 4310 |
| PS-18 | KNG1_205_5402 | 13 | 27 | 3617.47 | 205 | 9 | 5402 |
| PS-19 | IGM_209_5412 | 12 | 28 | 4688.90 | 209 | 7 | 5412 |

TABLE 1B
Peptide Structures associated with NPC

| | | | | | Linking | Linking | |
|---|---|---|---|---|---|---|---|
| | | (Protein) | (Peptide) | Mono- | Site Pos. in | Site Pos. in | Glycan |
| PS-ID | Peptide Structure | SEQ ID | SEQ ID | isotopic | Protein | Peptide | Structure |
| NO. | (PS) NAME | NO. | NO. | mass (Da) | Sequence | Sequence | GL NO. |
| PS-20 | A1AT_107_6503 | 29 | 41 | 6551.81 | 107 | 14 | 6503 |
| PS-21 | A1AT_107_6513 | 29 | 41 | 6697.87 | 107 | 14 | 6513 |
| PS-3 | A2MG_55_5411 | 2 | 15 | 4455.96 | 55 | 9 | 5411 |
| PS-22 | C4BPA_221_6503 | 30 | 42 | 6232.62 | 221 | 15 | 6503 |
| PS-23 | CERU_138_6513 | 31 | 43 | 4898.89 | 138 | 10 | 6513 |
| PS-24 | CO2_621_5200 | 32 | 44 | 2670.19 | 621 | 11 | 5200 |
| PS-25 | HEMO_187_6513 | 33 | 45 | 4410.71 | 187 | 7 | 6513 |
| PS-26 | HRG_125_5401 | 34 | 46 | 3927.64 | 125 | 5 | 5401 |
| PS-27 | IC1_238_5402 | 35 | 47 | 3113.20 | 238 | 5 | 5402 |
| PS-28 | IC1_253_6513 | 35 | 48 | 5107.14 | 253 | 4 | 6513 |
| PS-8 | IGA1_144_3500 | 5 | 20 | 4464.14 | 144 | 18 | 3500 |
| PS-9 | IGA2_131_3500 | 6 | 20 | 4464.14 | 131 | 18 | 3500 |
| PS-29 | IGG1_297_3300 | 36 | 49 | 2283.90 | 297 | 5 | 3300 |
| PS-30 | IGG1_297_3410 | 36 | 49 | 2633.038 | 297 | 5 | 3410 |
| PS-31 | IGG1_297_4400 | 36 | 49 | 2649.03 | 297 | 5 | 4400 |
| PS-32 | IGG2_297_4410 | 37 | 50 | 2763.10 | 297 | 5 | 4410 |
| PS-33 | KLKB1_127_5401 | 38 | 51 | 2777.12 | 127 | 5 | 5401 |
| PS-34 | TRFE_630_5402_{—} | 39 | 52 | 4701.86 | 630 | 9 | 5402 |
| | NH3LOSS | | | | | | |
| PS-35 | VTNC_86_5402 | 40 | 53 | 4584.93 | 86 | 2 | 5402 |

Tables 1A and 1B includes the Peptide Structure Identification Number (PS-ID NO.) that is a reference number for a particular peptide or glycopeptide. The Peptide Structure Name (PS-Name, e.g., IC1_238_5402), which is a reference code for the protein name (e.g., IC1), followed by the glycan linking site position in the protein (e.g., the number 238 that is in between two underscores and represents a sequential amino acid position in protein IC1), and followed by the glycan structure GL number (e.g., the number 5402 that is preceded by an underscore and represents a glycan composition Hex(5)HexNAc(4)Fuc(0)NeuAc(2)). The Protein Sequence ID No's of Tables 1A and 1B correspond to the protein name, Unitprot ID, and amino acid sequence of Tables 6A and 6B. The Peptide Sequence ID No's of Tables 1A and 1B correspond to the peptide sequence of Tables 5A and 5B, respectively. The term Linking Site Pos. within Protein Sequence is a number that refers to the sequential position of an amino acid of the corresponding protein in which a glycan is attached. For the Glycan Linking Site Pos. within Protein Sequence, the amino acid position of the peptide sequence is defined by the sequentially numbered order of amino acids based on the Uniprot ID of the corresponding protein for the peptide sequence. The term Linking Site Pos. within Peptide Sequence is a number that refers to the sequential position of an amino acid of the corresponding peptide in which a glycan is attached. For the Glycan Linking Site Pos. in peptide Sequence, the amino acid position of the peptide sequence is defined by the sequentially numbered order of amino acids for the peptide sequence. The term Glycan Structure GL No. is a number that corresponds to a symbol structure and a composition of the glycan as indicated in Table 7A and 7B.

In some instances of the Peptide Structure (PS) NAME, subsequent to the prefix, there is a number noted with the notation MC that indicates that there was a missed cleavage at position in the peptide sequence as noted by the number. In some instances of the Peptide Structure (PS) NAME, there is a suffix NH₃LOSS to indicate a loss of a NH₃ group from the precursor ion.

### VI.B. Training the Model to Diagnose with Respect to the NPC State

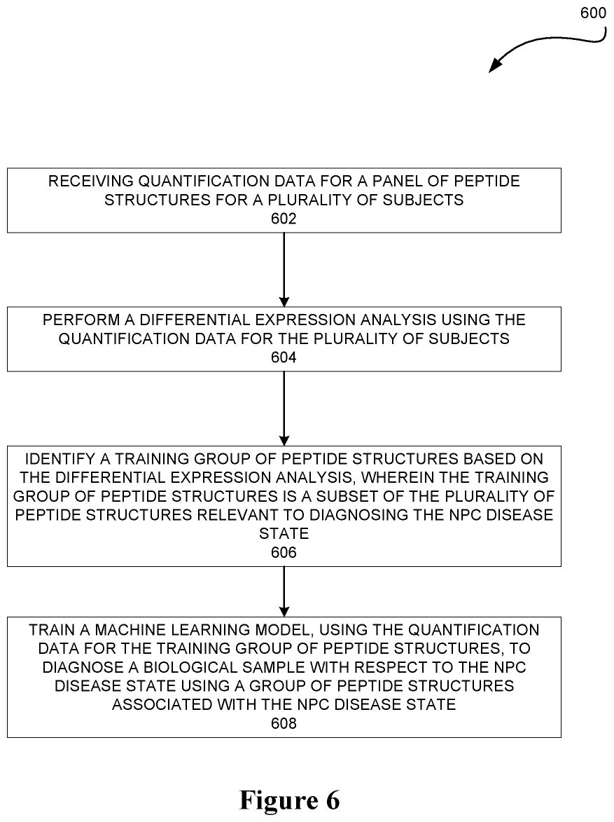

FIG. 6 is a flowchart of a process for training a model to diagnose a subject with respect to a nasopharyngeal carcinoma (NPC) disease state in accordance with one or more embodiments. Process 600 may be implemented using, for example, at least a portion of workflow 100 as described in FIGS. 1, 2A, and 2B and/or analysis system 300 as described in FIG. 3. In some embodiments, process 600 may be one example of an implementation for training the model used in the process 500 in FIG. 5.

Step 602 includes receiving quantification data for a panel of peptide structures for a plurality of subjects. The plurality of subjects includes a first portion diagnosed with the NPC disease state and a second portion diagnosed with a non-NPC state. The quantification data comprises a plurality of peptide structure profiles for the plurality of subjects.

Step 604 includes performing a differential expression analysis using the quantification data for the plurality of subjects.

Step 606 includes identifying a training group of peptide structures based on the differential expression analysis, wherein the training group of peptide structures is a subset of the plurality of peptide structures relevant to diagnosing the NPC disease state. The subset may be identified based on at least one of fold-changes, false discovery rates, or p-values computed as part of the differential expression analysis.

Step 608 includes training a machine learning model, using the quantification data for the training group of peptide structures, to diagnose a biological sample with respect to the NPC disease state using a group of peptide structures associated with the NPC disease state. The group of peptide structures may be a subset of the training group of peptide structures and is identified in Table 1A and/or 1B. The group of peptide structures is listed in Table 1A and/or 1B with respect to relative significance to making the diagnosis.

In various embodiments, the machine learning model is a supervised machine learning model that is trained to determine weight coefficients for a panel of peptide structures such that a first portion of the weight coefficients for a first portion of the panel of peptide structures are non-zero and a second portion of the weight coefficients for a second portion of the panel of peptide structures are zero, with the first portion of the panel of peptide structures forming the group of peptide structures identified in Table 1A and/or 1B.

For example, the machine learning model may be a LASSO regression model that identifies coefficients such as those in Table 2A and 2B below for peptide structure markers that include at least a portion of the group of peptide structures identified in Table 1A and 1B, respectively. The markers used for training of the LASSO regression model may, in one or more embodiments, additionally include one or more other peptide structure markers.

TABLE 2A
Peptide Structures and Associated Coefficients

| Model | | | (Protein) | (Peptide) | |
|---|---|---|---|---|---|
| Marker | PS-ID | | SEQ ID | SEQ ID | LASSO |
| Index | NO. | Peptide Structure (PS) NAME | NO. | NO. | Coefficients |
| 1 | PS-1 | APOC3_74_1300 | 1 | 14 | 0.8910260 |
| 2 | PS-2 | APOC3_74_1102 | 1 | 14 | 0.1155582 |
| 3 | PS-3 | A2MG_55_5411 | 2 | 15 | −0.0232025 |
| 4 | PS-4 | APOC3_74MC_1102 | 1 | 16 | 0.0142774 |
| 5 | PS-5 | A2MG_1424_5402_Z3 | 2 | 17 | 0.2233073 |
| 6 | PS-6 | KLKB1_494_5400 | 3 | 18 | 0.0840061 |
| 7 | PS-7 | APOB_983_5401 | 4 | 19 | −0.5753620 |
| 8 | PS-8 | IGA1_144_3500 | 5 | 20 | 0.2192268 |
| | and/or | IGA2_131_3500 | 6 | | |
| | PS-9 | | | | |
| 9 | PS-10 | TRFE_432_5402 | 7 | 21 | −0.3917101 |
| 10 | PS-11 | FETUA_156_5400 | 8 | 22 | −0.0796909 |
| 11 | PS-12 | KLKB1_494_6503 | 3 | 18 | 0.1456771 |
| 12 | PS-13 | A2MG_1424_5412 | 2 | 17 | −0.2702506 |
| 13 | PS-14 | AACT_106_7604 | 9 | 23 | −0.0077225 |
| 14 | PS-15 | VTNC_86_6503 | 10 | 24 | −0.1516314 |
| 15 | PS-16 | HEP2_49_5412 | 11 | 25 | 0.0346340 |
| 16 | PS-17 | IGM_46_4310 | 12 | 26 | 0.0413282 |
| 17 | PS-18 | KNG1_205_5402 | 13 | 27 | −0.1436711 |
| 18 | PS-19 | IGM_209_5412 | 12 | 28 | 0.0312812 |

TABLE 2B
Peptide Structures and Associated Coefficients

| Model | | | (Protein) | (Peptide) | |
|---|---|---|---|---|---|
| Marker | PS-ID | | SEQ ID | SEQ ID | LASSO |
| Index | NO. | Peptide Structure (PS) NAME | NO. | NO. | Coefficients |
| 1 | PS-20 | A1AT_107_6503 | 29 | 41 | 0.07280802 |
| 2 | PS-21 | A1AT_107_6513 | 29 | 41 | 0.30549532 |
| 3 | PS-3 | A2MG_55_5411 | 2 | 15 | −0.53570570 |
| 4 | PS-22 | C4BPA_221_6503 | 30 | 42 | 0.05331411 |
| 5 | PS-23 | CERU_138_6513 | 31 | 43 | 0.03192483 |
| 6 | PS-24 | CO2_621_5200 | 32 | 44 | 1.97521749 |
| 7 | PS-25 | HEMO_187_6513 | 33 | 45 | 0.01220036 |
| 8 | PS-26 | HRG_125_5401 | 34 | 46 | −0.24111950 |
| 9 | PS-27 | IC1_238_5402 | 35 | 47 | 0.02761670 |
| 10 | PS-28 | IC1_253_6513 | 35 | 48 | 0.20146409 |
| 11 | PS-8 | IGA12_144_3500 | 5 | 20 | 0.02751795 |
| | and/or | | and/or | | |
| | PS-9 | | 6 | | |
| 12 | PS-29 | IGG1_297_3300 | 36 | 49 | 2.13863077 |
| 13 | PS-30 | IGG1_297_3410 | 36 | 49 | 0.02084467 |
| 14 | PS-31 | IGG1_297_4400 | 36 | 49 | 0.09289814 |
| 15 | PS-32 | IGG2_297_4410 | 37 | 50 | −0.02315294 |
| 16 | PS-33 | KLKB1_127_5401 | 38 | 51 | −1.01528504 |
| 17 | PS-34 | TRFE_630_5402_NH3LOSS | 39 | 52 | −0.16513091 |
| 18 | PS-35 | VTNC_86_5402 | 40 | 53 | −0.18623468 |

In one or more embodiments, the peptide structures identified in Table 2A and/or 2B may be only a subset of the markers used for training of the LASSO regression model. For example, the LASSO regression model may be trained using at least one other marker in addition to those identified in Table 2A and/or 2B. In training the LASSO regression model, for model marker index 8, the quantification metrics for peptide structure PS-8, peptide structure PS-9, or a combination of the two may be used.

Table 3A and 3B below includes the fold changes, FDRs, and p-values determined using the differential expression analysis (DEA) for markers that include at least a portion of the group of peptide structures identified in Table 1A and 1B, respectively. In performing the differential expression analysis, for DEA marker index 8, the quantification metrics for peptide structure PS-8, peptide structure PS-9, or a combination of the two may be used.

TABLE 3A
Differential Expression Analysis (DEA) for NPC v. Control

| DEA | | | | | |
|---|---|---|---|---|---|
| Marker | PS-ID | | NPC/Control | NPC/Control | NPC/Control |
| Index | NO. | PS-NAME | (fold change) | (FDR) | (p-value) |
| 1 | PS-1 | APOC3_74_1300 | 1.45426 | 1.0078E−07 | 2.4702E−10 |
| 2 | PS-2 | APOC3_74_1102 | 1.21434 | 8.2569E−05 | 6.0713E−07 |
| 3 | PS-3 | A2MG_55_5411 | 0.95514 | 2.1136E−04 | 3.7547E−06 |
| 4 | PS-4 | APOC3_74MC_1102 | 1.13543 | 2.1136E−04 | 4.1444E−06 |
| 5 | PS-5 | A2MG_1424_5402_Z3 | 1.13501 | 2.3358E−04 | 7.4424E−06 |
| 6 | PS-6 | KLKB1_494_5400 | 1.17939 | 3.4666E−04 | 1.5294E−05 |
| 7 | PS-7 | APOB_983_5401 | 0.94923 | 5.3391E−04 | 3.2265E−05 |
| 8 | PS-8 | IGA1_144_3500 | 1.02704 | 5.4624E−04 | 3.8826E−05 |
| | and/or | IGA2_131_3500 | | | |
| | PS-9 | | | | |
| 9 | PS-9 | IGA2_131_3500 | 1.02704 | 5.4624E−04 | 3.8826E−05 |
| 9 | PS-10 | TRFE_432_5402 | 0.93755 | 1.4943E−03 | 1.4650E−04 |
| 10 | PS-11 | FETUA_156_5400 | 0.85453 | 4.3992E−03 | 5.6893E−04 |
| 11 | PS-12 | KLKB1_494_6503 | 1.14152 | 4.7015E−03 | 6.2226E−04 |
| 12 | PS-13 | A2MG_1424_5412 | 0.95763 | 5.2618E−02 | 1.3928E−02 |
| 13 | PS-14 | AACT_106_7604 | 0.95388 | 6.4474E−02 | 1.9121E−02 |
| 14 | PS-15 | VTNC_86_6503 | 0.94468 | 7.2856E−02 | 2.3035E−02 |
| 15 | PS-16 | HEP2_49_5412 | 1.02287 | 1.8759E−01 | 8.3682E−02 |
| 16 | PS-17 | IGM_46_4310 | 1.02120 | 3.4822E−01 | 1.9289E−01 |
| 17 | PS-18 | KNG1_205_5402 | 0.99373 | 7.9377E−01 | 6.6765E−01 |
| 18 | PS-19 | IGM_209_5412 | 1.00473 | 8.2495E−01 | 7.2148E−01 |

TABLE 3B
Differential Expression Analysis (DEA) for NPC v. Control

| DEA | | | | | |
|---|---|---|---|---|---|
| Marker | PS-ID | | NPC/Control | NPC/Control | NPC/Control |
| Index | NO. | PS-NAME | (fold change) | (FDR) | (p-value) |
| 1 | PS-20 | A1AT_107_6503 | 1.314 | 3.19E−12 | 2.95E−11 |
| 2 | PS-21 | A1AT_107_6513 | 1.866 | 7.04E−17 | 1.80E−15 |
| 3 | PS-3 | A2MG_55_5411 | 0.832 | 6.90E−14 | 9.77E−13 |
| 4 | PS-22 | C4BPA_221_6503 | 1.253 | 7.01E−07 | 2.19E−06 |
| 5 | PS-23 | CERU_138_6513 | 1.426 | 7.88E−12 | 6.41E−11 |
| 6 | PS-24 | CO2_621_5200 | 1.43 | 1.07E−14 | 1.69E−13 |
| 7 | PS-25 | HEMO_187_6513 | 1.475 | 2.37E−14 | 3.63E−13 |
| 8 | PS-26 | HRG_125_5401 | 0.827 | 8.12E−08 | 2.99E−07 |
| 9 | PS-27 | IC1_238_5402 | 1.165 | 4.37E−10 | 2.45E−09 |
| 10 | PS-28 | IC1_253_6513 | 1.518 | 8.62E−15 | 1.45E−13 |
| 11 | PS-8 | IGA12_144_3500 | 1.647 | 1.18E−10 | 7.53E−10 |
| | and/or | | | | |
| | PS-9 | | | | |
| 12 | PS-29 | IGG1_297_3300 | 2.98 | 1.71E−36 | 9.17E−34 |
| 13 | PS-30 | IGG1_297_3410 | 1.923 | 3.84E−28 | 6.87E−26 |
| 14 | PS-31 | IGG1_297_4400 | 1.869 | 4.03E−24 | 3.61E−22 |
| 15 | PS-32 | IGG2_297_4410 | 0.726 | 3.76E−12 | 3.42E−11 |
| 16 | PS-33 | KLKB1_127_5401 | 0.76 | 5.77E−08 | 2.18E−07 |
| 17 | PS-34 | TRFE_630_5402_NH3LOSS | 0.794 | 6.42E−11 | 4.42E−10 |
| 18 | PS-35 | VTNC_86_5402 | 0.859 | 5.35E−07 | 1.69E−06 |

### VI.C. Monitoring a Progression of NPC

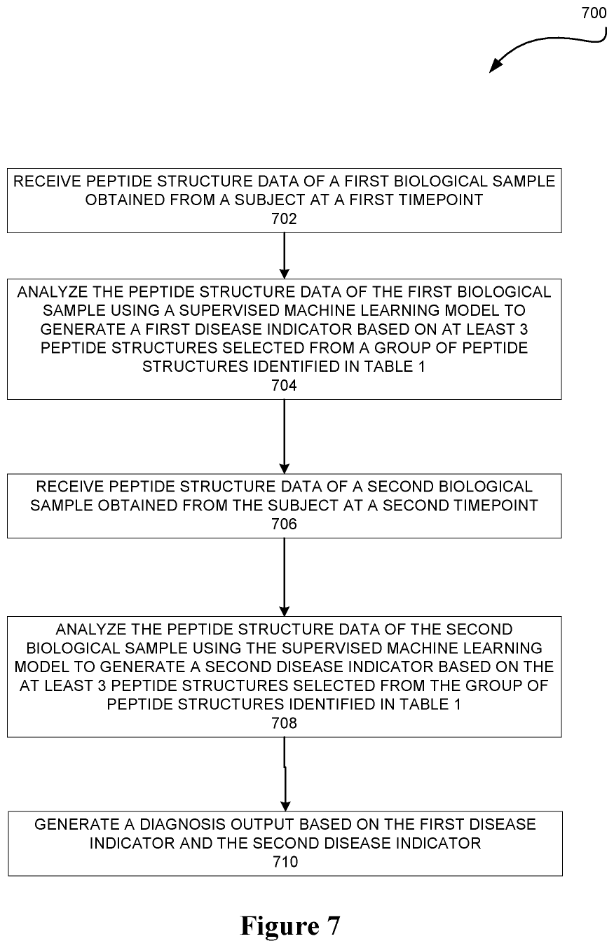

FIG. 7 is a flowchart of a process for monitoring a subject for a nasopharyngeal carcinoma (NPC) in accordance with one or more embodiments. Process 700 may be implemented using, for example, at least a portion of workflow 100 as described in FIGS. 1, 2A, and 2B and/or analysis system 300 as described in FIG. 3.

Step 702 includes receiving peptide structure data of a first biological sample obtained from a subject at a first timepoint.

Step 704 includes analyzing the peptide structure data of the first biological sample using a supervised machine learning model to generate a first disease indicator based on at least 3 peptide structures selected from a group of peptide structures identified in Table 1. The group of peptide structures in Table 1A and/or 1B comprises a group of peptide structures associated with the NPC disease state.

Step 706 includes receiving peptide structure data of a second biological sample obtained from the subject at a second timepoint.

Step 708 includes analyzing the peptide structure data of the second biological sample using the supervised machine learning model to generate a second disease indicator based on the at least 3 peptide structures selected from the group of peptide structures identified in Table 1.

Step 710 includes generating a diagnosis output based on the first disease indicator and the second disease indicator. In one or more embodiments, step 710 is performed by comparing the disease indicators. The first disease indicator may indicate that the first biological sample evidences the NPC disease state and the second disease may indicate that the second biological sample does not evidence the NPC disease state. In such cases, the diagnosis output may indicate that the subject no longer has NPC and/or that a treatment administered to the subject after the first biological sample was obtained but before the second biological sample was obtained is effective. The method described in FIG. 7 using Table 1 is applicable to either Table 1A and/or 1B.

### VII. Exemplary Methodology for Treating NPC

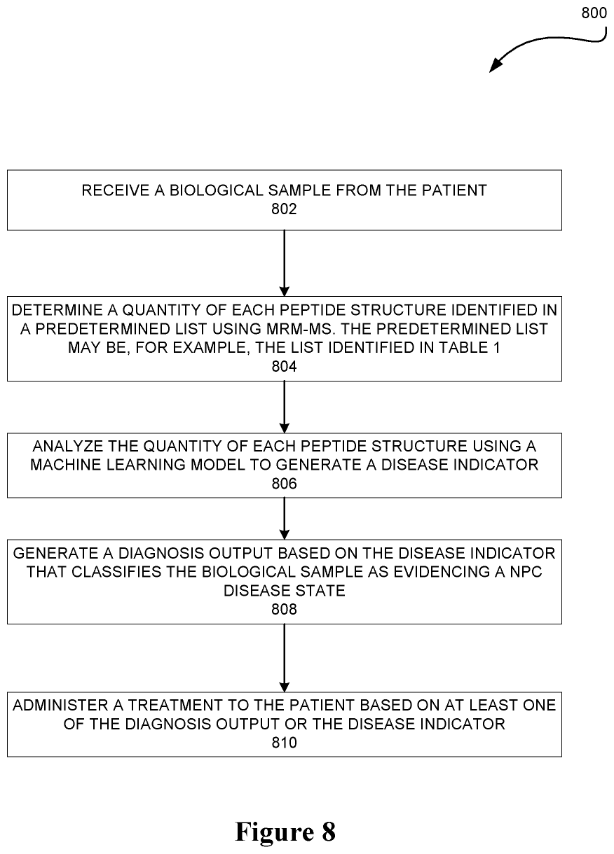

FIG. 8 is a flowchart of a process for treating a subject for HCC in accordance with one or more embodiments. Process 800 may be at least partially implemented using at least a portion of workflow 80 as described FIGS. 1, 2A, and/or 2B and/or analysis system 300 as described in FIG. 3.

Step 802 includes receiving a biological sample from the patient.

Step 804 includes determining a quantity of each peptide structure identified in a predetermined list using MRM-MS. The predetermined list may be, for example, the list identified in Table 1A and/or 1B determining a quantity of each peptide structure identified in a predetermined list using MRM-MS. The predetermined list may be, for example, the list identified in Table 1A and/or 1B or the list identified in Table 2A and/or 2B.

Step 806 includes analyzing the quantity of each peptide structure using a machine learning model to generate a disease indicator.

Step 808 includes generating a diagnosis output based on the disease indicator that classifies the biological sample as evidencing a NPC disease state.

Step 810 includes administering a treatment to the patient based on at least one of the diagnosis output or the disease indicator. The treatment may be, for example, a radiation treatment that is administered via at least one of intravenous or oral administration of the treatment at therapeutic dosage(s). In some embodiments, the treatment includes chemoradiation, surgery, a targeted drug therapy, or a combination thereof. For example, radiation may be used to treat stage 0 or stage 1 NPC. Chemoradiotherapy may be used to treat stage II, III, and IV NPC.

In one or more embodiments, process 800 may additionally include a step before step 810 or that is part of step 810 in which a treatment output is generated based on at least one of the diagnosis output or the disease indicator. The treatment output may identify the treatment that is to be administered to the patient, a therapeutic dosage and/or regimen for the treatment, and/or other information. The method described in FIG. 8 using Table 1 is applicable to either Table 1A and/or 1B.

### VIII. Peptide Structure and Product Ion Compositions, Kits and Reagents

Aspects of the disclosure include compositions comprising one or more of the peptide structures listed in Table 1A and/or 1B. In some embodiments, a composition comprises a plurality of the peptide structures listed in Table 1A and/or 1B. In some embodiments, a composition comprises 1, 2, 3, 4, 5, 6, 7, 8, 9, 10, 11, 12, 13, 14, 15, 16, 17, 18, or 19 of the peptide structures listed in Table 1A and/or comprises 1, 2, 3, 4, 5, 6, 7, 8, 9, 10, 11, 12, 13, 14, 15, 16, 17, 18, or 19 of the peptide structures listed in Table 1B. In some embodiments, a composition comprises a peptide structure having an amino acid sequence with at least 80% sequence identity, such as, for example, at least 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NOs: 14-28, listed in Table 1A and/or a composition comprises a peptide structure having an amino acid sequence with at least 80% sequence identity, such as, for example, at least 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NOs: 15, 20, or 41-53, listed in Table 1B.

Aspects of the disclosure include compositions comprising one or more precursor ions having a defined charge and/or defined mass-to-charge (m/z) ratio, as listed in Table 4A or 4B. Aspects of the disclosure include compositions comprising one or more product ions having a defined mass-to-charge (m/z) ratio, which product ions are produced by converting a peptide structure described herein (e.g., a peptide structure listed in Table 1) into a gas phase ion in a mass spectrometry system. Conversion of the peptide structure into a gas phase ion can take place using any of a variety of techniques, including, but not limited to, matrix assisted laser desorption ionization (MALDI); electron ionization (EI); electrospray ionization (ESI); atmospheric pressure chemical ionization (APCI); and/or atmospheric pressure photo ionization (APPI).

Aspects of the disclosure include compositions comprising one or more product ions produced from one or more of the peptide structures described herein (e.g., a peptide structure listed in Table 1A or 1B). In some embodiments, a composition comprises a set of the product ions listed in Table 4A or 4B, having an m/z ratio selected from the list provided for each peptide structure in Table 1A or 1B or Table 4A or 4B.

In some embodiments, a composition comprises at least one of peptide structures PS-1 to PS-19 identified in Table 1A and/or a composition comprises at least one of peptide structures PS-3, PS-8, PS-9, and PS-20 to PS-35 identified in Table 1B.

In some embodiments, a composition comprises a peptide structure or a product ion. The peptide structure or product ion comprises an amino acid sequence having at least 90% sequence identity to any one of SEQ ID NOS: 14-28, as identified in Table 5A, corresponding to peptide structures PS-1 to PS-19 in Table 1A, and/or the peptide structure or product ion comprises an amino acid sequence having at least 90% sequence identity to any one of SEQ ID NOS: 15, 20, and 41-53, as identified in Table 5B, corresponding to peptide structures PS-3, PS-8, PS-9, and PS-20 to PS-35 in Table 1B.

In some embodiments, the product ion is selected as one from a group consisting of product ions identified in Table 4A and/or 4B, including product ions falling within an identified m/z range of the m/z ratio identified in Table 4A and/or 4B and characterized as having a precursor ion having an m/z ratio within an identified m/z range of the m/z ratio identified in Table 4A and/or 4B. A first range for the product ion m/z ratio may be ±0.5. A second range for the product ion m/z ratio may be ±0.8. A third range for the product ion m/z ratio may be ±1.0. A first range for the precursor ion m/z ratio may be ±1.0; a second range for the precursor ion m/z ratio may be (±1.5). Thus, a composition may include a product ion having an m/z ratio that falls within at least one of the first range (±0.5), the second range (±0.8), or the third range (±1.0) of the product ion m/z ratio identified in Table 4A and/or 4B, and characterized as having a precursor ion having an m/z ratio that falls within at least one of first range (±0.5), a second range (±1.0), or a third range (±1.0 of the precursor ion m/z ratio identified in Table 4A and/or 4B.

Tables 4A and 4B show various parameters associated with the identification of the peptide and glycopeptides using LC and MRM-MS. The retention time (RT) represents the amount of time in minutes for the peptide to elute from the chromatography column. The collision energy represents the energy applied to the peptide for creating fragments (i.e., product ions) such as, for example, in the 2^{nd} quadrupole of the triple quadrupole MS. The first precursor m/z represents a ratio value associated with an ionized form having a precursor charge for the peptide or glycopeptide. The precursor ion is associated with a first product ion having a m/z ratio and a second product ion having a m/z ratio that were both formed from the collision.

TABLE 4A
Mass Spectrometry-Related Characteristics for
the Peptide Structures associated with NPC

| | | | | | 1st | 1st | 2nd | 2nd |
|---|---|---|---|---|---|---|---|---|
| PS-ID | RT | Collision | Precursor | Precursor | Product | Product | Product | Product |
| NO. | (min) | Energy | m/z | Charge | m/z | Charge | m/z | Charge |
| PS-1 | 37.5 | 23 | 970.1 | 3 | 366.1 | 1 | N/A | N/A |
| PS-2 | 38.2 | 20/25 | 1028.8 | 3 | 274.1 | 1 | 1069 | 2 |
| PS-3 | 41.5 | 22 | 1115.4 | 4 | 366.1 | 1 | N/A | N/A |
| PS-4 | 37.8 | 27 | 1109.8 | 3 | 274.1 | 1 | 1190 | 2 |
| PS-5 | 44.1 | 30/30 | 1457.3 | 3 | 366.1 | 1 | 1183.6 | 2 |
| PS-6 | 30.9 | 30 | 968.2 | 4 | 366.1 | 1 | N/A | N/A |
| PS-7 | 33.2 | 25 | 1367.6 | 4 | 366.1 | 1 | N/A | N/A |
| PS-8 | 40.3 | 27 | 1117.1 | 4 | 204.1 | 1 | N/A | N/A |
| PS-9 | 40.3 | 27 | 1117.1 | 4 | 204.1 | 1 | N/A | N/A |
| PS-10 | 27.3 | 22 | 921.4 | 4 | 366.1 | 1 | N/A | N/A |
| PS-11 | 26.5 | 30 | 1132.2 | 3 | 366.1 | 1 | N/A | N/A |
| PS-12 | 33.6 | 38 | 1277.8 | 4 | 366.1 | 1 | N/A | N/A |
| PS-13 | 44.1 | 23/30 | 1129.7 | 4 | 366.1 | 1 | 1183.6 | 2 |
| PS-14 | 38.4 | 30 | 1184.9 | 5 | 274.1 | 1 | N/A | N/A |
| PS-15 | 19.4 | 37/25 | 1311.8 | 4 | 366.1 | 1 | 1292.6 | 2 |
| PS-16 | 32.2 | 38 | 1278.5 | 3 | 366.1 | 1 | N/A | N/A |
| PS-17 | 6.3 | 30/30 | 896.7 | 3 | 204.1 | 1 | 744.4 | 2 |
| PS-18 | 17.1 | 20 | 905.6 | 4 | 366.1 | 1 | N/A | N/A |
| PS-19 | 26.1 | 29 | 1173.3 | 4 | 366.1 | 1 | N/A | N/A |

TABLE 4B
Mass Spectrometry-Related Characteristics for
the Peptide Structures associated with NPC

| | | | | | 1st | 1st | 2nd | 2nd |
|---|---|---|---|---|---|---|---|---|
| PS-ID | RT | Collision | Precursor | Precursor | Product | Product | Product | Product |
| NO. | (min) | Energy | m/z | Charge | m/z | Charge | m/z | Charge |
| PS-20 | 43.2 | 33 | 1311.8 | 5 | 366.1 | 1 | N/A | N/A |
| PS-21 | 43.17 | 34 | 1341 | 5 | 366.1 | 1 | N/A | N/A |
| PS-3 | 41.36 | 22 | 1115.4 | 4 | 366.1 | 1 | 366.1 | 1 |
| PS-22 | 37.13 | 33 | 1248.1 | 5 | 366.1 | 1 | N/A | N/A |
| PS-23 | 17.18 | 30 | 1226.2 | 4 | 366.1 | 1 | N/A | N/A |
| PS-24 | 16.08 | 20 | 891.1 | 3 | 829.4 | 1 | N/A | N/A |
| PS-25 | 21.58 | 25 | 1104.2 | 4 | 274.1 | 1 | N/A | N/A |
| PS-26 | 27.53 | 20 | 983.4 | 4 | 366.1 | 1 | N/A | N/A |
| PS-27 | 10.17 | 25 | 1039.1 | 3 | 366.1 | 1 | N/A | N/A |
| PS-28 | 35.84 | 40 | 1278.3 | 4 | 204.1 | 1 | N/A | N/A |
| PS-8 | 40.12 | 27 | 1117.1 | 4 | 204.1 | 1 | N/A | N/A |
| and/or | | | | | | | | |
| PS-9 | | | | | | | | |
| PS-29 | 7.86 | 30 | 1143.5 | 2 | 204.1 | 1 | N/A | N/A |
| PS-30 | 7.85 | 21 | 879 | 3 | 204.1 | 1 | N/A | N/A |
| PS-31 | 7.82 | 21 | 884.4 | 3 | 204.1 | 1 | N/A | N/A |
| PS-32 | 12.78 | 22 | 922.1 | 3 | 204.1 | 1 | N/A | N/A |
| PS-33 | 13.08 | 25 | 927 | 3 | 366.1 | 1 | N/A | N/A |
| PS-34 | 35.14 | 29 | 1177 | 4 | 366.1 | 1 | N/A | N/A |
| PS-35 | 18.71 | 35 | 1148 | 4 | 366.1 | 1 | N/A | N/A |

Table 5A defines the peptide sequences for SEQ ID NOS: 14-28 from Table 1A. Table 5A further identifies a corresponding protein SEQ ID NO. for each peptide sequence.

TABLE 5A
Peptide SEQ ID NOS

| | | Corresponding |
|---|---|---|
| SEQ ID | | Protein |
| NO: | Peptide Sequence | SEQ ID NO: |
| 14 | FSEFWDLDPEVRPTSAVAA | 1 |
| 15 | GCVLLSYLNETVTVSASLESVR | 2 |
| 16 | DKFSEFWDLDPEVRPTSAVAA | 1 |
| 17 | VSNQTLSLFFTVLQDVPVR | 2 |
| 18 | LQAPLNYTEFQKPICLPSK | 3 |
| 19 | QVFPGLNYCTSGAYSNASSTDSASYYPLTGDTR | 4 |
| 20 | LSLHRPALEDLLLGSEANLTCTLTGLR | 5, 6 |
| 21 | CGLVPVLAENYNK | 7 |
| 22 | VCQDCPLLAPLNDTR | 8 |
| 23 | FNLTETSEAEIHQSFQHLLR | 9 |
| 24 | NNATVHEQVGGPSLTSDLQAQSK | 10 |
| 25 | NLSMPLLPADFHK | 11 |
| 26 | YKNNSDISSTR | 12 |
| 27 | ITYSIVQTNCSK | 13 |
| 28 | GLTFQQNASSMCVPDQDTAIR | 12 |

Table 5B defines the peptide sequences for SEQ ID NOS: 15, 20, and 41-53 from Table 1B. Table 5B further identifies a corresponding protein SEQ ID NOs: 2, 5, 6, and 29-40 for each peptide sequence.

TABLE 5B
Peptide SEQ ID NOS

| | | Corresponding |
|---|---|---|
| SEQ ID | | Protein |
| NO: | Peptide Sequence | SEQ ID NO: |
| 41 | ADTHDEILEGLNFNLTEIPEAQIHEGFQELLR | 29 |
| 15 | GCVLLSYLNETVTVSASLESVR | 2 |
| 42 | FSLLGHASISCTVENETIGVWRPSPPTCEK | 30 |
| 43 | EHEGAIYPDNTTDFQR | 31 |
| 44 | QSVPAHFVALNGSK | 32 |
| 45 | SWPAVGNCSSALR | 33 |
| 46 | VIDFNCTTSSVSSALANTK | 34 |
| 47 | DTFVNASR | 35 |
| 48 | VLSNNSDANLELINTWVAK | 35 |
| 20 | LSLHRPALEDLLLGSEANLTCTLTGLR | 5 |
| | | and/or |
| | | 6 |
| 49 | EEQYNSTYR | 36 |
| 50 | EEQFNSTFR | 37 |
| 51 | GVNFNVSK | 38 |
| 52 | QQQHLFGSNVTDCSGNFCLFR | 39 |
| 53 | NNATVHEQVGGPSLTSDLQAQSK | 40 |

Table 6A identifies the proteins of SEQ ID NOS: 1-13 from Table 1A. Table 6A identifies a corresponding protein abbreviation and protein name for each of protein SEQ ID NOS: 1-13. Further, Table 6A identifies a corresponding Uniprot ID for each of protein SEQ ID NOS: 1-13.

TABLE 6A
Protein SEQ ID NOS

| SEQ | | | | |
|---|---|---|---|---|
| ID | Protein | | Uniprot | |
| NO. | Abbreviation | Protein Name | ID | Protein Sequence |
| 1 | APOC3 | Apolipoprotein | P02656 | MQPRVLLVVALLALLASARASEAEDASLL |
| | | C-III | | SFMQGYMKHATKTAKDALSSVQESQVAQ |
| | | | | QARGWVTDGFSSLKDYWSTVKDKFSEFW |
| | | | | DLDPEVRPTSAVAA |
| 2 | A2MG | Alpha-2- | P01023 | MGKNKLLHPSLVLLLLVLLPTDASVSGKP |
| | | macroglobulin | | QYMVLVPSLLHTETTEKGCVLLSYLNETV |
| | | | | TVSASLESVRGNRSLFTDLEAENDVLHCV |
| | | | | AFAVPKSSSNEEVMFLTVQVKGPTQEFKK |
| | | | | RTTVMVKNEDSLVFVQTDKSIYKPGQTVK |
| | | | | FRVVSMDENFHPLNELIPLVYIQDPKGNRI |
| | | | | AQWQSFQLEGGLKQFSFPLSSEPFQGSYKV |
| | | | | VVQKKSGGRTEHPFTVEEFVLPKFEVQVT |
| | | | | VPKIITILEEEMNVSVCGLYTYGKPVPGHV |
| | | | | TVSICRKYSDASDCHGEDSQAFCEKFSGQL |
| | | | | NSHGCFYQQVKTKVFQLKRKEYEMKLHT |
| | | | | EAQIQEEGTVVELTGRQSSEITRTITKLSFV |
| | | | | KVDSHFRQGIPFFGQVRLVDGKGVPIPNKV |
| | | | | IFIRGNEANYYSNATTDEHGLVQFSINTTN |
| | | | | VMGTSLTVRVNYKDRSPCYGYQWVSEEH |
| | | | | EEAHHTAYLVFSPSKSFVHLEPMSHELPCG |
| | | | | HTQTVQAHYILNGGTLLGLKKLSFYYLIM |
| | | | | AKGGIVRTGTHGLLVKQEDMKGHFSISIPV |
| | | | | KSDIAPVARLLIYAVLPTGDVIGDSAKYDV |
| | | | | ENCLANKVDLSFSPSQSLPASHAHLRVTA |
| | | | | APQSVCALRAVDQSVLLMKPDAELSASSV |
| | | | | YNLLPEKDLTGFPGPLNDQDNEDCINRHN |
| | | | | VYINGITYTPVSSTNEKDMYSFLEDMGLK |
| | | | | AFTNSKIRKPKMCPQLQQYEMHGPEGLRV |
| | | | | GFYESDVMGRGHARLVHVEEPHTETVRK |
| | | | | YFPETWIWDLVVVNSAGVAEVGVTVPDTI |
| | | | | TEWKAGAFCLSEDAGLGISSTASLRAFQPF |
| | | | | FVELTMPYSVIRGEAFTLKATVLNYLPKCI |
| | | | | RVSVQLEASPAFLAVPVEKEQAPHCICAN |
| | | | | GRQTVSWAVTPKSLGNVNFTVSAEALESQ |
| | | | | ELCGTEVPSVPEHGRKDTVIKPLLVEPEGL |
| | | | | EKETTFNSLLCPSGGEVSEELSLKLPPNVV |
| | | | | EESARASVSVLGDILGSAMQNTQNLLQMP |
| | | | | YGCGEQNMVLFAPNIYVLDYLNETQQLTP |
| | | | | EIKSKAIGYLNTGYQRQLNYKHYDGSYST |
| | | | | FGERYGRNQGNTWLTAFVLKTFAQARAYI |
| | | | | FIDEAHITQALIWLSQRQKDNGCFRSSGSL |
| | | | | LNNAIKGGVEDEVTLSAYITIALLEIPLTVT |
| | | | | HPVVRNALFCLESAWKTAQEGDHGSHVY |
| | | | | TKALLAYAFALAGNQDKRKEVLKSLNEE |
| | | | | AVKKDNSVHWERPQKPKAPVGHFYEPQA |
| | | | | PSAEVEMTSYVLLAYLTAQPAPTSEDLTSA |
| | | | | TNIVKWITKQQNAQGGFSSTQDTVVALHA |
| | | | | LSKYGAATFTRTGKAAQVTIQSSGTFSSKF |
| | | | | QVDNNNRLLLQQVSLPELPGEYSMKVTGE |
| | | | | GCVYLQTSLKYNILPEKEEFPFALGVQTLP |
| | | | | QTCDEPKAHTSFQISLSVSYTGSRSASNMA |
| | | | | IVDVKMVSGFIPLKPTVKMLERSNHVSRTE |
| | | | | VSSNHVLIYLDKVSNQTLSLFFTVLQDVPV |
| | | | | RDLKPAIVKVYDYYETDEFAIAEYNAPCS |
| | | | | KDLGNA |
| 3 | KLKB1 | Plasma | P03952 | MILFKQATYFISLFATVSCGCLTQLYENAF |
| | | Kallikrein | | FRGGDVASMYTPNAQYCQMRCTFHPRCL |
| | | | | LFSFLPASSINDMEKRFGCFLKDSVTGTLP |
| | | | | KVHRTGAVSGHSLKQCGHQISACHRDIYK |
| | | | | GVDMRGVNFNVSKVSSVEECQKRCTNNIR |
| | | | | CQFFSYATQTFHKAEYRNNCLLKYSPGGT |
| | | | | PTAIKVLSNVESGFSLKPCALSEIGCHMNIF |
| | | | | QHLAFSDVDVARVLTPDAFVCRTICTYHP |
| | | | | NCLFFTFYTNVWKIESQRNVCLLKTSESGT |
| | | | | PSSSTPQENTISGYSLLTCKRTLPEPCHSKIY |
| | | | | PGVDFGGEELNVTFVKGVNVCQETCTKMI |
| | | | | RCQFFTYSLLPEDCKEEKCKCFLRLSMDGS |
| | | | | PTRIAYGTQGSSGYSLRLCNTGDNSVCTTK |
| | | | | TSTRIVGGTNSSWGEWPWQVSLQVKLTA |
| | | | | QRHLCGGSLIGHQWVLTAAHCFDGLPLQD |
| | | | | VWRIYSGILNLSDITKDTPFSQIKEIIIHQNY |
| | | | | KVSEGNHDIALIKLQAPLNYTEFQKPICLPS |
| | | | | KGDTSTIYTNCWVTGWGFSKEKGEIQNIL |
| | | | | QKVNIPLVTNEECQKRYQDYKITQRMVCA |
| | | | | GYKEGGKDACKGDSGGPLVCKHNGMWR |
| | | | | LVGITSWGEGCARREQPGVYTKVAEYMD |
| | | | | WILEKTQSSDGKAQMQSPA |
| 4 | APOB | Apolipoprotein | P04114 | MDPPRPALLALLALPALLLLLLAGARAEEE |
| | | B-100 | | MLENVSLVCPKDATRFKHLRKYTYNYEA |
| | | | | ESSSGVPGTADSRSATRINCKVELEVPQLC |
| | | | | SFILKTSQCTLKEVYGFNPEGKALLKKTKN |
| | | | | SEEFAAAMSRYELKLAIPEGKQVFLYPEKD |
| | | | | EPTYILNIKRGIISALLVPPETEEAKQVLFLD |
| | | | | TVYGNCSTHFTVKTRKGNVATEISTERDL |
| | | | | GQCDRFKPIRTGISPLALIKGMTRPLSTLISS |
| | | | | SQSCQYTLDAKRKHVAEAICKEQHLFLPFS |
| | | | | YKNKYGMVAQVTQTLKLEDTPKINSRFFG |
| | | | | EGTKKMGLAFESTKSTSPPKQAEAVLKTL |
| | | | | QELKKLTISEQNIQRANLFNKLVTELRGLS |
| | | | | DEAVTSLLPQLIEVSSPITLQALVQCGQPQC |
| | | | | STHILQWLKRVHANPLLIDVVTYLVALIPE |
| | | | | PSAQQLREIFNMARDQRSRATLYALSHAV |
| | | | | NNYHKTNPTGTQELLDIANYLMEQIQDDC |
| | | | | TGDEDYTYLILRVIGNMGQTMEQLTPELK |
| | | | | SSILKCVQSTKPSLMIQKAAIQALRKMEPK |
| | | | | DKDQEVLLQTFLDDASPGDKRLAAYLML |
| | | | | MRSPSQADINKIVQILPWEQNEQVKNFVAS |
| | | | | HIANILNSEELDIQDLKKLVKEALKESQLPT |
| | | | | VMDFRKFSRNYQLYKSVSLPSLDPASAKIE |
| | | | | GNLIFDPNNYLPKESMLKTTLTAFGFASAD |
| | | | | LIEIGLEGKGFEPTLEALFGKQGFFPDSVNK |
| | | | | ALYWVNGQVPDGVSKVLVDHFGYTKDD |
| | | | | KHEQDMVNGIMLSVEKLIKDLKSKEVPEA |
| | | | | RAYLRILGEELGFASLHDLQLLGKLLLMG |
| | | | | ARTLQGIPQMIGEVIRKGSKNDFFLHYIFM |
| | | | | ENAFELPTGAGLQLQISSSGVIAPGAKAGV |
| | | | | KLEVANMQAELVAKPSVSVEFVTNMGIIIP |
| | | | | DFARSGVQMNTNFFHESGLEAHVALKAG |
| | | | | KLKFIIPSPKRPVKLLSGGNTLHLVSTTKTE |
| | | | | VIPPLIENRQSWSVCKQVFPGLNYCTSGAY |
| | | | | SNASSTDSASYYPLTGDTRLELELRPTGEIE |
| | | | | QYSVSATYELQREDRALVDTLKFVTQAEG |
| | | | | AKQTEATMTFKYNRQSMTLSSEVQIPDFD |
| | | | | VDLGTILRVNDESTEGKTSYRLTLDIQNKK |
| | | | | ITEVALMGHLSCDTKEERKIKGVISIPRLQA |
| | | | | EARSEILAHWSPAKLLLQMDSSATAYGST |
| | | | | VSKRVAWHYDEEKIEFEWNTGTNVDTKK |
| | | | | MTSNFPVDLSDYPKSLHMYANRLLDHRVP |
| | | | | QTDMTFRHVGSKLIVAMSSWLQKASGSLP |
| | | | | YTQTLQDHLNSLKEFNLQNMGLPDFHIPE |
| | | | | NLFLKSDGRVKYTLNKNSLKIEIPLPFGGK |
| | | | | SSRDLKMLETVRTPALHFKSVGFHLPSREF |
| | | | | QVPTFTIPKLYQLQVPLLGVLDLSTNVYSN |
| | | | | LYNWSASYSGGNTSTDHFSLRARYHMKA |
| | | | | DSVVDLLSYNVQGSGETTYDHKNTFTLSY |
| | | | | DGSLRHKFLDSNIKFSHVEKLGNNPVSKGL |
| | | | | LIFDASSSWGPQMSASVHLDSKKKQHLFV |
| | | | | KEVKIDGQFRVSSFYAKGTYGLSCQRDPN |
| | | | | TGRLNGESNLRFNSSYLQGTNQITGRYED |
| | | | | GTLSLTSTSDLQSGIIKNTASLKYENYELTL |
| | | | | KSDTNGKYKNFATSNKMDMTFSKQNALL |
| | | | | RSEYQADYESLRFFSLLSGSLNSHGLELNA |
| | | | | DILGTDKINSGAHKATLRIGQDGISTSATTN |
| | | | | LKCSLLVLENELNAELGLSGASMKLTTNG |
| | | | | RFREHNAKFSLDGKAALTELSLGSAYQAM |
| | | | | ILGVDSKNIFNFKVSQEGLKLSNDMMGSY |
| | | | | AEMKFDHTNSLNIAGLSLDFSSKLDNIYSS |
| | | | | DKFYKQTVNLQLQPYSLVTTLNSDLKYNA |
| | | | | LDLTNNGKLRLEPLKLHVAGNLKGAYQN |
| | | | | NEIKHIYAISSAALSASYKADTVAKVQGVE |
| | | | | FSHRLNTDIAGLASAIDMSTNYNSDSLHFS |
| | | | | NVFRSVMAPFTMTIDAHTNGNGKLALWG |
| | | | | EHTGQLYSKFLLKAEPLAFTFSHDYKGSTS |
| | | | | HHLVSRKSISAALEHKVSALLTPAEQTGT |
| | | | | WKLKTQFNNNEYSQDLDAYNTKDKIGVE |
| | | | | LTGRTLADLTLLDSPIKVPLLLSEPINIIDAL |
| | | | | EMRDAVEKPQEFTIVAFVKYDKNQDVHSI |
| | | | | NLPFFETLQEYFERNRQTIIVVLENVQRNL |
| | | | | KHINIDQFVRKYRAALGKLPQQANDYLNS |
| | | | | FNWERQVSHAKEKLTALTKKYRITENDIQI |
| | | | | ALDDAKINFNEKLSQLQTYMIQFDQYIKDS |
| | | | | YDLHDLKIAIANIIDEIIEKLKSLDEHYHIRV |
| | | | | NLVKTIHDLHLFIENIDENKSGSSTASWIQN |
| | | | | VDTKYQIRIQIQEKLQQLKRHIQNIDIQHLA |
| | | | | GKLKQHIEAIDVRVLLDQLGTTISFERINDI |
| | | | | LEHVKHFVINLIGDFEVAEKINAFRAKVHE |
| | | | | LIERYEVDQQIQVLMDKLVELAHQYKLKE |
| | | | | TIQKLSNVLQQVKIKDYFEKLVGFIDDAVK |
| | | | | KLNELSFKTFIEDVNKFLDMLIKKLKSFDY |
| | | | | HQFVDETNDKIREVTQRLNGEIQALELPQK |
| | | | | AEALKLFLEETKATVAVYLESLQDTKITLII |
| | | | | NWLQEALSSASLAHMKAKFRETLEDTRDR |
| | | | | MYQMDIQQELQRYLSLVGQVYSTLVTYIS |
| | | | | DWWTLAAKNLTDFAEQYSIQDWAKRMK |
| | | | | ALVEQGFTVPEIKTILGTMPAFEVSLQALQ |
| | | | | KATFQTPDFIVPLTDLRIPSVQINFKDLKNI |
| | | | | KIPSRFSTPEFTILNTFHIPSFTIDFVEMKVKI |
| | | | | IRTIDQMLNSELQWPVPDIYLRDLKVEDIP |
| | | | | LARITLPDFRLPEIAIPEFIIPTLNLNDFQVPD |
| | | | | LHIPEFQLPHISHTIEVPTFGKLYSILKIQSPL |
| | | | | FTLDANADIGNGTTSANEAGIAASITAKGE |
| | | | | SKLEVLNFDFQANAQLSNPKINPLALKESV |
| | | | | KFSSKYLRTEHGSEMLFFGNAIEGKSNTVA |
| | | | | SLHTEKNTLELSNGVIVKINNQLTLDSNTK |
| | | | | YFHKLNIPKLDFSSQADLRNEIKTLLKAGH |
| | | | | IAWTSSGKGSWKWACPRFSDEGTHESQISF |
| | | | | TIEGPLTSFGLSNKINSKHLRVNQNLVYES |
| | | | | GSLNFSKLEIQSQVDSQHVGHSVLTAKGM |
| | | | | ALFGEGKAEFTGRHDAHLNGKVIGTLKNS |
| | | | | LFFSAQPFEITASTNNEGNLKVRFPLRLTG |
| | | | | KIDFLNNYALFLSPSAQQASWQVSARFNQ |
| | | | | YKYNQNFSAGNNENIMEAHVGINGEANL |
| | | | | DFLNIPLTIPEMRLPYTIITTPPLKDFSLWEK |
| | | | | TGLKEFLKTTKQSFDLSVKAQYKKNKHRH |
| | | | | SITNPLAVLCEFISQSIKSFDRHFEKNRNNA |
| | | | | LDFVTKSYNETKIKFDKYKAEKSHDELPRT |
| | | | | FQIPGYTVPVVNVEVSPFTIEMSAFGYVFP |
| | | | | KAVSMPSFSILGSDVRVPSYTLILPSLELPV |
| | | | | LHVPRNLKLSLPDFKELCTISHIFIPAMGNI |
| | | | | TYDFSFKSSVITLNTNAELFNQSDIVAHLLS |
| | | | | SSSSVIDALQYKLEGTTRLTRKRGLKLATA |
| | | | | LSLSNKFVEGSHNSTVSLTTKNMEVSVAT |
| | | | | TTKAQIPILRMNFKQELNGNTKSKPTVSSS |
| | | | | MEFKYDFNSSMLYSTAKGAVDHKLSLESL |
| | | | | TSYFSIESSTKGDVKGSVLSREYSGTIASEA |
| | | | | NTYLNSKSTRSSVKLQGTSKIDDIWNLEVK |
| | | | | ENFAGEATLQRIYSLWEHSTKNHLQLEGL |
| | | | | FFTNGEHTSKATLELSPWQMSALVQVHAS |
| | | | | QPSSFHDFPDLGQEVALNANTKNQKIRWK |
| | | | | NEVRIHSGSFQSQVELSNDQEKAHLDIAGS |
| | | | | LEGHLRFLKNIILPVYDKSLWDFLKLDVTT |
| | | | | SIGRRQHLRVSTAFVYTKNPNGYSFSIPVK |
| | | | | VLADKFIIPGLKLNDLNSVLVMPTFHVPFT |
| | | | | DLQVPSCKLDFREIQIYKKLRTSSFALNLPT |
| | | | | LPEVKFPEVDVLTKYSQPEDSLIPFFEITVP |
| | | | | ESQLTVSQFTLPKSVSDGIAALDLNAVANK |
| | | | | IADFELPTIIVPEQTIEIPSIKFSVPAGIVIPSF |
| | | | | QALTARFEVDSPVYNATWSASLKNKADY |
| | | | | VETVLDSTCSSTVQFLEYELNVLGTHKIED |
| | | | | GTLASKTKGTFAHRDFSAEYEEDGKYEGL |
| | | | | QEWEGKAHLNIKSPAFTDLHLRYQKDKK |
| | | | | GISTSAASPAVGTVGMDMDEDDDFSKWN |
| | | | | FYYSPQSSPDKKLTIFKTELRVRESDEETQI |
| | | | | KVNWEEEAASGLLTSLKDNVPKATGVLY |
| | | | | DYVNKYHWEHTGLTLREVSSKLRRNLQN |
| | | | | NAEWVYQGAIRQIDDIDVRFQKAASGTTG |
| | | | | TYQEWKDKAQNLYQELLTQEGQASFQGL |
| | | | | KDNVFDGLVRVTQEFHMKVKHLIDSLIDF |
| | | | | LNFPRFQFPGKPGIYTREELCTMFIREVGTV |
| | | | | LSQVYSKVHNGSEILFSYFQDLVITLPFELR |
| | | | | KHKLIDVISMYRELLKDLSKEAQEVFKAIQ |
| | | | | SLKTTEVLRNLQDLLQFIFQLIEDNIKQLKE |
| | | | | MKFTYLINYIQDEINTIFSDYIPYVFKLLKE |
| | | | | NLCLNLHKFNEFIQNELQEASQELQQIHQY |
| | | | | IMALREEYFDPSIVGWTVKYYELEEKIVSLI |
| | | | | KNLLVALKDFHSEYIVSASNFTSQLSSQVE |
| | | | | QFLHRNIQEYLSILTDPDGKGKEKIAELSAT |
| | | | | AQEIIKSQAIATKKIISDYHQQFRYKLQDFS |
| | | | | DQLSDYYEKFIAESKRLIDLSIQNYHTFLIYI |
| | | | | TELLKKLQSTTVMNPYMKLAPGELTIIL |
| 5 | IGA1 | Immunoglobulin | P01876 | ASPTSPKVFPLSLCSTQPDGNVVIACLVQG |
| | | heavy | | FFPQEPLSVTWSESGQGVTARNFPPSQDAS |
| | | constant alpha | | GDLYTTSSQLTLPATQCLAGKSVTCHVKH |
| | | 1 | | YTNPSQDVTVPCPVPSTPPTPSPSTPPTPSPS |
| | | | | CCHPRLSLHRPALEDLLLGSEANLTCTLTG |
| | | | | LRDASGVTFTWTPSSGKSAVQGPPERDLC |
| | | | | GCYSVSSVLPGCAEPWNHGKTFTCTAAYP |
| | | | | ESKTPLTATLSKSGNTFRPEVHLLPPPSEEL |
| | | | | ALNELVTLTCLARGFSPKDVLVRWLQGSQ |
| | | | | ELPREKYLTWASRQEPSQGTTTFAVTSILR |
| | | | | VAAEDWKKGDTFSCMVGHEALPLAFTQK |
| | | | | TIDRLAGKPTHVNVSVVMAEVDGTCY |
| 6 | IGA2 | Immunoglobulin | P01877 | ASPTSPKVFPLSLDSTPQDGNVVVACLVQ |
| | | heavy | | GFFPQEPLSVTWSESGQNVTARNFPPSQDA |
| | | constant alpha | | SGDLYTTSSQLTLPATQCPDGKSVTCHVK |
| | | 2 | | HYTNSSQDVTVPCRVPPPPPCCHPRLSLHR |
| | | | | PALEDLLLGSEANLTCTLTGLRDASGATFT |
| | | | | WTPSSGKSAVQGPPERDLCGCYSVSSVLP |
| | | | | GCAQPWNHGETFTCTAAHPELKTPLTANI |
| | | | | TKSGNTFRPEVHLLPPPSEELALNELVTLT |
| | | | | CLARGFSPKDVLVRWLQGSQELPREKYLT |
| | | | | WASRQEPSQGTTTYAVTSILRVAAEDWKK |
| | | | | GETFSCMVGHEALPLAFTQKTIDRMAGKP |
| | | | | THINVSVVMAEADGTCY |
| 7 | TRFE | Serotransferrin | P02787 | MRLAVGALLVCAVLGLCLAVPDKTVRWC |
| | | | | AVSEHEATKCQSFRDHMKSVIPSDGPSVA |
| | | | | CVKKASYLDCIRAIAANEADAVTLDAGLV |
| | | | | YDAYLAPNNLKPVVAEFYGSKEDPQTFYY |
| | | | | AVAVVKKDSGFQMNQLRGKKSCHTGLGR |
| | | | | SAGWNIPIGLLYCDLPEPRKPLEKAVANFF |
| | | | | SGSCAPCADGTDFPQLCQLCPGCGCSTLN |
| | | | | QYFGYSGAFKCLKDGAGDVAFVKHSTIFE |
| | | | | NLANKADRDQYELLCLDNTRKPVDEYKD |
| | | | | CHLAQVPSHTVVARSMGGKEDLIWELLN |
| | | | | QAQEHFGKDKSKEFQLFSSPHGKDLLFKD |
| | | | | SAHGFLKVPPRMDAKMYLGYEYVTAIRN |
| | | | | LREGTCPEAPTDECKPVKWCALSHHERLK |
| | | | | CDEWSVNSVGKIECVSAETTEDCIAKIMN |
| | | | | GEADAMSLDGGFVYIAGKCGLVPVLAEN |
| | | | | YNKSDNCEDTPEAGYFAIAVVKKSASDLT |
| | | | | WDNLKGKKSCHTAVGRTAGWNIPMGLLY |
| | | | | NKINHCRFDEFFSEGCAPGSKKDSSLCKLC |
| | | | | MGSGLNLCEPNNKEGYYGYTGAFRCLVE |
| | | | | KGDVAFVKHQTVPQNTGGKNPDPWAKNL |
| | | | | NEKDYELLCLDGTRKPVEEYANCHLARAP |
| | | | | NHAVVTRKDKEACVHKILRQQQHLFGSN |
| | | | | VTDCSGNFCLFRSETKDLLFRDDTVCLAK |
| | | | | LHDRNTYEKYLGEEYVKAVGNLRKCSTSS |
| | | | | LLEACTFRRP |
| 8 | FETUA | Alpha-2-HS- | P02765 | MKSLVLLLCLAQLWGCHSAPHGPGLIYRQ |
| | | glycoprotein | | PNCDDPETEEAALVAIDYINQNLPWGYKH |
| | | | | TLNQIDEVKVWPQQPSGELFEIEIDTLETTC |
| | | | | HVLDPTPVARCSVRQLKEHAVEGDCDFQL |
| | | | | LKLDGKFSVVYAKCDSSPDSAEDVRKVCQ |
| | | | | DCPLLAPLNDTRVVHAAKAALAAFNAQN |
| | | | | NGSNFQLEEISRAQLVPLPPSTYVEFTVSGT |
| | | | | DCVAKEATEAAKCNLLAEKQYGFCKATL |
| | | | | SEKLGGAEVAVTCMVFQTQPVSSQPQPEG |
| | | | | ANEAVPTPVVDPDAPPSPPLGAPGLPPAGS |
| | | | | PPDSHVLLAAPPGHQLHRAHYDLRHTFMG |
| | | | | VVSLGSPSGEVSHPRKTRTVVQPSVGAAA |
| | | | | GPVVPPCPGRIRHFKV |
| 9 | AACT | Alpha-1- | P01011 | MERMLPLLALGLLAAGFCPAVLCHPNSPL |
| | | antichymotrypsin | | DEENLTQENQDRGTHVDLGLASANVDFAF |
| | | | | SLYKQLVLKAPDKNVIFSPLSISTALAFLSL |
| | | | | GAHNTTLTEILKGLKFNLTETSEAEIHQSFQ |
| | | | | HLLRTLNQSSDELQLSMGNAMFVKEQLSL |
| | | | | LDRFTEDAKRLYGSEAFATDFQDSAAAKK |
| | | | | LINDYVKNGTRGKITDLIKDLDSQTMMVL |
| | | | | VNYIFFKAKWEMPFDPQDTHQSRFYLSKK |
| | | | | KWVMVPMMSLHHLTIPYFRDEELSCTVVE |
| | | | | LKYTGNASALFILPDQDKMEEVEAMLLPE |
| | | | | TLKRWRDSLEFREIGELYLPKFSISRDYNL |
| | | | | NDILLQLGIEEAFTSKADLSGITGARNLAVS |
| | | | | QVVHKAVLDVFEEGTEASAATAVKITLLS |
| | | | | ALVETRTIVRFNRPFLMIIVPTDTQNIFFMS |
| | | | | KVTNPKQA |
| 10 | VTNC | Vitronectin | P04004 | MAPLRPLLILALLAWVALADQESCKGRCT |
| | | | | EGFNVDKKCQCDELCSYYQSCCTDYTAEC |
| | | | | KPQVTRGDVFTMPEDEYTVYDDGEEKNN |
| | | | | ATVHEQVGGPSLTSDLQAQSKGNPEQTPV |
| | | | | LKPEEEAPAPEVGASKPEGIDSRPETLHPG |
| | | | | RPQPPAEEELCSGKPFDAFTDLKNGSLFAF |
| | | | | RGQYCYELDEKAVRPGYPKLIRDVWGIEG |
| | | | | PIDAAFTRINCQGKTYLFKGSQYWRFEDG |
| | | | | VLDPDYPRNISDGFDGIPDNVDAALALPAH |
| | | | | SYSGRERVYFFKGKQYWEYQFQHQPSQEE |
| | | | | CEGSSLSAVFEHFAMMQRDSWEDIFELLF |
| | | | | WGRTSAGTRQPQFISRDWHGVPGQVDAA |
| | | | | MAGRIYISGMAPRPSLAKKQRFRHRNRKG |
| | | | | YRSQRGHSRGRNQNSRRPSRATWLSLFSS |
| | | | | EESNLGANNYDDYRMDWLVPATCEPIQSV |
| | | | | FFFSGDKYYRVNLRTRRVDTVDPPYPRSIA |
| | | | | QYWLGCPAPGHL |
| 11 | HEP2 | Heparin | P05546 | MKHSLNALLIFLIITSAWGGSKGPLDQLEK |
| | | cofactor 2 | | GGETAQSADPQWEQLNNKNLSMPLLPAD |
| | | | | FHKENTVTNDWIPEGEEDDDYLDLEKIFSE |
| | | | | DDDYIDIVDSLSVSPTDSDVSAGNILQLFH |
| | | | | GKSRIQRLNILNAKFAFNLYRVLKDQVNTF |
| | | | | DNIFIAPVGISTAMGMISLGLKGETHEQVH |
| | | | | SILHFKDFVNASSKYEITTIHNLFRKLTHRL |
| | | | | FRRNFGYTLRSVNDLYIQKQFPILLDFKTK |
| | | | | VREYYFAEAQIADFSDPAFISKTNNHIMKL |
| | | | | TKGLIKDALENIDPATQMMILNCIYFKGSW |
| | | | | VNKFPVEMTHNHNFRLNEREVVKVSMMQ |
| | | | | TKGNFLAANDQELDCDILQLEYVGGISMLI |
| | | | | VVPHKMSGMKTLEAQLTPRVVERWQKSM |
| | | | | TNRTREVLLPKFKLEKNYNLVESLKLMGI |
| | | | | RMLFDKNGNMAGISDQRIAIDLFKHQGTIT |
| | | | | VNEEGTQATTVTTVGFMPLSTQVRFTVDR |
| | | | | PFLFLIYEHRTSCLLFMGRVANPSRS |
| 12 | IGM | Immunoglobulin | P01871 | GSASAPTLFPLVSCENSPSDTSSVAVGCLA |
| | | heavy | | QDFLPDSITFSWKYKNNSDISSTRGFPSVLR |
| | | constant mu | | GGKYAATSQVLLPSKDVMQGTDEHVVCK |
| | | | | VQHPNGNKEKNVPLPVIAELPPKVSVFVPP |
| | | | | RDGFFGNPRKSKLICQATGFSPRQIQVSWL |
| | | | | REGKQVGSGVTTDQVQAEAKESGPTTYK |
| | | | | VTSTLTIKESDWLGQSMFTCRVDHRGLTF |
| | | | | QQNASSMCVPDQDTAIRVFAIPPSFASIFLT |
| | | | | KSTKLTCLVTDLTTYDSVTISWTRQNGEA |
| | | | | VKTHTNISESHPNATFSAVGEASICEDDWN |
| | | | | SGERFTCTVTHTDLPSPLKQTISRPKGVAL |
| | | | | HRPDVYLLPPAREQLNLRESATITCLVTGF |
| | | | | SPADVFVQWMQRGQPLSPEKYVTSAPMPE |
| | | | | PQAPGRYFAHSILTVSEEEWNTGETYTCV |
| | | | | VAHEALPNRVTERTVDKSTGKPTLYNVSL |
| | | | | VMSDTAGTCY |
| 13 | KNG1 | Kininogen-1 | P01042 | MKLITILFLCSRLLLSLTQESQSEEIDCNDK |
| | | | | DLFKAVDAALKKYNSQNQSNNQFVLYRIT |
| | | | | EATKTVGSDTFYSFKYEIKEGDCPVQSGKT |
| | | | | WQDCEYKDAAKAATGECTATVGKRSSTK |
| | | | | FSVATQTCQITPAEGPVVTAQYDCLGCVH |
| | | | | PISTQSPDLEPILRHGIQYFNNNTQHSSLFM |
| | | | | LNEVKRAQRQVVAGLNFRITYSIVQTNCS |
| | | | | KENFLFLTPDCKSLWNGDTGECTDNAYIDI |
| | | | | QLRIASFSQNCDIYPGKDFVQPPTKICVGCP |
| | | | | RDIPTNSPELEETLTHTITKLNAENNATFYF |
| | | | | KIDNVKKARVQVVAGKKYFIDFVARETTC |
| | | | | SKESNEELTESCETKKLGQSLDCNAEVYV |
| | | | | VPWEKKIYPTVNCQPLGMISLMKRPPGFSP |
| | | | | FRSSRIGEIKEETTVSPPHTSMAPAQDEERD |
| | | | | SGKEQGHTRRHDWGHEKQRKHNLGHGH |
| | | | | KHERDQGHGHQRGHGLGHGHEQQHGLG |
| | | | | HGHKFKLDDDLEHQGGHVLDHGHKHKH |
| | | | | GHGHGKHKNKGKKNGKHNGWKTEHLAS |
| | | | | SSEDSTTPSAQTQEKTEGPTPIPSLAKPGVT |
| | | | | VTFSDFQDSDLIATMMPPISPAPIQSDDDWI |
| | | | | PDIQIDPNGLSFNPISDFPDTTSPKCPGRPW |
| | | | | KSVSEINPTTQMKESYYFDLTDGLS |

Table 6A identifies the proteins of SEQ ID NOS: 1-13 from Table 1A. Table 6A identifies a corresponding protein abbreviation and protein name for each of protein SEQ ID NOS: 1-13. Further, Table 6A identifies a corresponding Uniprot ID for each of protein SEQ ID NOS: 1-13.
Table 6B identifies the proteins of SEQ ID NOS: 2, 5, 6, and 29-40 from Table 1B. Table 6B identifies a corresponding protein abbreviation and protein name for each of protein SEQ ID NOS: 2, 5, 6, and 29-40. Further, Table 6B identifies a corresponding Uniprot ID for each of protein SEQ ID NOS: 2, 5, 6, and 29-40 along with the respective amino acid protein sequence.

TABLE 6B
Protein SEQ ID NOS

| SEQ ID | Protein | | Uniprot | |
|---|---|---|---|---|
| NO. | Abbreviation | Protein Name | ID | Protein Sequence |
| 29 | A1AT | Alpha-1- | P01009 | MPSSVSWGILLLAGLCCLVPVSLAEDPQG |
| | | antitrypsin | | DAAQKTDTSHHDQDHPTFNKITPNLAEFA |
| | | | | FSLYRQLAHQSNSTNIFFSPVSIATAFAML |
| | | | | SLGTKADTHDEILEGLNFNLTEIPEAQIHE |
| | | | | GFQELLRTLNQPDSQLQLTTGNGLFLSEG |
| | | | | LKLVDKFLEDVKKLYHSEAFTVNFGDTEE |
| | | | | AKKQINDYVEKGTQGKIVDLVKELDRDT |
| | | | | VFALVNYIFFKGKWERPFEVKDTEEEDFH |
| | | | | VDQVTTVKVPMMKRLGMFNIQHCKKLSS |
| | | | | WVLLMKYLGNATAIFFLPDEGKLQHLEN |
| | | | | ELTHDIITKFLENEDRRSASLHLPKLSITGT |
| | | | | YDLKSVLGQLGITKVFSNGADLSGVTEEA |
| | | | | PLKLSKAVHKAVLTIDEKGTEAAGAMFL |
| | | | | EAIPMSIPPEVKFNKPFVFLMIEQNTKSPLF |
| | | | | MGKVVNPTQK |
| 2 | A2MG | Alpha-2- | P01023 | MGKNKLLHPSLVLLLLVLLPTDASVSGKP |
| | | macroglobulin | | QYMVLVPSLLHTETTEKGCVLLSYLNETV |
| | | | | TVSASLESVRGNRSLFTDLEAENDVLHCV |
| | | | | AFAVPKSSSNEEVMFLTVQVKGPTQEFKK |
| | | | | RTTVMVKNEDSLVFVQTDKSIYKPGQTV |
| | | | | KFRVVSMDENFHPLNELIPLVYIQDPKGN |
| | | | | RIAQWQSFQLEGGLKQFSFPLSSEPFQGSY |
| | | | | KVVVQKKSGGRTEHPFTVEEFVLPKFEVQ |
| | | | | VTVPKIITILEEEMNVSVCGLYTYGKPVPG |
| | | | | HVTVSICRKYSDASDCHGEDSQAFCEKFS |
| | | | | GQLNSHGCFYQQVKTKVFQLKRKEYEM |
| | | | | KLHTEAQIQEEGTVVELTGRQSSEITRTIT |
| | | | | KLSFVKVDSHFRQGIPFFGQVRLVDGKGV |
| | | | | PIPNKVIFIRGNEANYYSNATTDEHGLVQF |
| | | | | SINTTNVMGTSLTVRVNYKDRSPCYGYQ |
| | | | | WVSEEHEEAHHTAYLVFSPSKSFVHLEPM |
| | | | | SHELPCGHTQTVQAHYILNGGTLLGLKKL |
| | | | | SFYYLIMAKGGIVRTGTHGLLVKQEDMK |
| | | | | GHFSISIPVKSDIAPVARLLIYAVLPTGDVI |
| | | | | GDSAKYDVENCLANKVDLSFSPSQSLPAS |
| | | | | HAHLRVTAAPQSVCALRAVDQSVLLMKP |
| | | | | DAELSASSVYNLLPEKDLTGFPGPLNDQD |
| | | | | NEDCINRHNVYINGITYTPVSSTNEKDMY |
| | | | | SFLEDMGLKAFTNSKIRKPKMCPQLQQYE |
| | | | | MHGPEGLRVGFYESDVMGRGHARLVHV |
| | | | | EEPHTETVRKYFPETWIWDLVVVNSAGV |
| | | | | AEVGVTVPDTITEWKAGAFCLSEDAGLGI |
| | | | | SSTASLRAFQPFFVELTMPYSVIRGEAFTL |
| | | | | KATVLNYLPKCIRVSVQLEASPAFLAVPV |
| | | | | EKEQAPHCICANGRQTVSWAVTPKSLGN |
| | | | | VNFTVSAEALESQELCGTEVPSVPEHGRK |
| | | | | DTVIKPLLVEPEGLEKETTFNSLLCPSGGE |
| | | | | VSEELSLKLPPNVVEESARASVSVLGDILG |
| | | | | SAMQNTQNLLQMPYGCGEQNMVLFAPNI |
| | | | | YVLDYLNETQQLTPEIKSKAIGYLNTGYQ |
| | | | | RQLNYKHYDGSYSTFGERYGRNQGNTW |
| | | | | LTAFVLKTFAQARAYIFIDEAHITQALIWL |
| | | | | SQRQKDNGCFRSSGSLLNNAIKGGVEDEV |
| | | | | TLSAYITIALLEIPLTVTHPVVRNALFCLES |
| | | | | AWKTAQEGDHGSHVYTKALLAYAFALA |
| | | | | GNQDKRKEVLKSLNEEAVKKDNSVHWE |
| | | | | RPQKPKAPVGHFYEPQAPSAEVEMTSYVL |
| | | | | LAYLTAQPAPTSEDLTSATNIVKWITKQQ |
| | | | | NAQGGFSSTQDTVVALHALSKYGAATFT |
| | | | | RTGKAAQVTIQSSGTFSSKFQVDNNNRLL |
| | | | | LQQVSLPELPGEYSMKVTGEGCVYLQTSL |
| | | | | KYNILPEKEEFPFALGVQTLPQTCDEPKA |
| | | | | HTSFQISLSVSYTGSRSASNMAIVDVKMV |
| | | | | SGFIPLKPTVKMLERSNHVSRTEVSSNHV |
| | | | | LIYLDKVSNQTLSLFFTVLQDVPVRDLKP |
| | | | | AIVKVYDYYETDEFAIAEYNAPCSKDLGN |
| | | | | A |
| 30 | C4BPA | C4b-binding | P04003 | MHPPKTPSGALHRKRKMAAWPFSRLWK |
| | | protein alpha | | VSDPILFQMTLIAALLPAVLGNCGPPPTLS |
| | | chain | | FAAPMDITLTETRFKTGTTLKYTCLPGYV |
| | | | | RSHSTQTLTCNSDGEWVYNTFCIYKRCRH |
| | | | | PGELRNGQVEIKTDLSFGSQIEFSCSEGFFL |
| | | | | IGSTTSRCEVQDRGVGWSHPLPQCEIVKC |
| | | | | KPPPDIRNGRHSGEENFYAYGFSVTYSCD |
| | | | | PRFSLLGHASISCTVENETIGVWRPSPPTC |
| | | | | EKITCRKPDVSHGEMVSGFGPIYNYKDTI |
| | | | | VFKCQKGFVLRGSSVIHCDADSKWNPSPP |
| | | | | ACEPNSCINLPDIPHASWETYPRPTKEDVY |
| | | | | VVGTVLRYRCHPGYKPTTDEPTTVICQKN |
| | | | | LRWTPYQGCEALCCPEPKLNNGEITQHRK |
| | | | | SRPANHCVYFYGDEISFSCHETSRFSAICQ |
| | | | | GDGTWSPRTPSCGDICNFPPKIAHGHYKQ |
| | | | | SSSYSFFKEEIIYECDKGYILVGQAKLSCS |
| | | | | YSHWSAPAPQCKALCRKPELVNGRLSVD |
| | | | | KDQYVEPENVTIQCDSGYGVVGPQSITCS |
| | | | | GNRTWYPEVPKCEWETPEGCEQVLTGKR |
| | | | | LMQCLPNPEDVKMALEVYKLSLEIEQLEL |
| | | | | QRDSARQSTLDKEL |
| 31 | CERU | Ceruloplasmin | P00450 | MKILILGIFLFLCSTPAWAKEKHYYIGIIET |
| | | | | TWDYASDHGEKKLISVDTEHSNIYLQNGP |
| | | | | DRIGRLYKKALYLQYTDETFRTTIEKPVW |
| | | | | LGFLGPIIKAETGDKVYVHLKNLASRPYT |
| | | | | FHSHGITYYKEHEGAIYPDNTTDFQRADD |
| | | | | KVYPGEQYTYMLLATEEQSPGEGDGNCV |
| | | | | TRIYHSHIDAPKDIASGLIGPLIICKKDSLD |
| | | | | KEKEKHIDREFVVMFSVVDENFSWYLED |
| | | | | NIKTYCSEPEKVDKDNEDFQESNRMYSV |
| | | | | NGYTFGSLPGLSMCAEDRVKWYLFGMG |
| | | | | NEVDVHAAFFHGQALTNKNYRIDTINLFP |
| | | | | ATLFDAYMVAQNPGEWMLSCQNLNHLK |
| | | | | AGLQAFFQVQECNKSSSKDNIRGKHVRH |
| | | | | YYIAAEEIIWNYAPSGIDIFTKENLTAPGSD |
| | | | | SAVFFEQGTTRIGGSYKKLVYREYTDASF |
| | | | | TNRKERGPEEEHLGILGPVIWAEVGDTIR |
| | | | | VTFHNKGAYPLSIEPIGVRFNKNNEGTYY |
| | | | | SPNYNPQSRSVPPSASHVAPTETFTYEWT |
| | | | | VPKEVGPTNADPVCLAKMYYSAVDPTKD |
| | | | | IFTGLIGPMKICKKGSLHANGRQKDVDKE |
| | | | | FYLFPTVFDENESLLLEDNIRMFTTAPDQV |
| | | | | DKEDEDFQESNKMHSMNGFMYGNQPGL |
| | | | | TMCKGDSVVWYLFSAGNEADVHGIYFSG |
| | | | | NTYLWRGERRDTANLFPQTSLTLHMWPD |
| | | | | TEGTFNVECLTTDHYTGGMKQKYTVNQC |
| | | | | RRQSEDSTFYLGERTYYIAAVEVEWDYSP |
| | | | | QREWEKELHHLQEQNVSNAFLDKGEFYI |
| | | | | GSKYKKVVYRQYTDSTFRVPVERKAEEE |
| | | | | HLGILGPQLHADVGDKVKIIFKNMATRPY |
| | | | | SIHAHGVQTESSTVTPTLPGETLTYVWKIP |
| | | | | ERSGAGTEDSACIPWAYYSTVDQVKDLY |
| | | | | SGLIGPLIVCRRPYLKVFNPRRKLEFALLF |
| | | | | LVFDENESWYLDDNIKTYSDHPEKVNKD |
| | | | | DEEFIESNKMHAINGRMFGNLQGLTMHV |
| | | | | GDEVNWYLMGMGNEIDLHTVHFHGHSF |
| | | | | QYKHRGVYSSDVFDIFPGTYQTLEMFPRT |
| | | | | PGIWLLHCHVTDHIHAGMETTYTVLQNE |
| | | | | DTKSG |
| 32 | CO2 | Complement C2 | P06681 | MGPLMVLFCLLFLYPGLADSAPSCPQNV |
| | | | | NISGGTFTLSHGWAPGSLLTYSCPQGLYPS |
| | | | | PASRLCKSSGQWQTPGATRSLSKAVCKP |
| | | | | VRCPAPVSFENGIYTPRLGSYPVGGNVSF |
| | | | | ECEDGFILRGSPVRQCRPNGMWDGETAV |
| | | | | CDNGAGHCPNPGISLGAVRTGFRFGHGD |
| | | | | KVRYRCSSNLVLTGSSERECQGNGVWSG |
| | | | | TEPICRQPYSYDFPEDVAPALGTSFSHML |
| | | | | GATNPTQKTKESLGRKIQIQRSGHLNLYL |
| | | | | LLDCSQSVSENDFLIFKESASLMVDRIFSF |
| | | | | EINVSVAIITFASEPKVLMSVLNDNSRDMT |
| | | | | EVISSLENANYKDHENGTGTNTYAALNSV |
| | | | | YLMMNNQMRLLGMETMAWQEIRHAIILL |
| | | | | TDGKSNMGGSPKTAVDHIREILNINQKRN |
| | | | | DYLDIYAIGVGKLDVDWRELNELGSKKD |
| | | | | GERHAFILQDTKALHQVFEHMLDVSKLT |
| | | | | DTICGVGNMSANASDQERTPWHVTIKPKS |
| | | | | QETCRGALISDQWVLTAAHCFRDGNDHS |
| | | | | LWRVNVGDPKSQWGKEFLIEKAVISPGFD |
| | | | | VFAKKNQGILEFYGDDIALLKLAQKVKM |
| | | | | STHARPICLPCTMEANLALRRPQGSTCRD |
| | | | | HENELLNKQSVPAHFVALNGSKLNINLK |
| | | | | MGVEWTSCAEVVSQEKTMFPNLTDVREV |
| | | | | VTDQFLCSGTQEDESPCKGESGGAVFLER |
| | | | | RFRFFQVGLVSWGLYNPCLGSADKNSRK |
| | | | | RAPRSKVPPPRDFHINLFRMQPWLRQHLG |
| | | | | DVLNFLPL |
| 33 | HEMO | Hemopexin | P02790 | MARVLGAPVALGLWSLCWSLAIATPLPPT |
| | | | | SAHGNVAEGETKPDPDVTERCSDGWSFD |
| | | | | ATTLDDNGTMLFFKGEFVWKSHKWDRE |
| | | | | LISERWKNFPSPVDAAFRQGHNSVFLIKG |
| | | | | DKVWVYPPEKKEKGYPKLLQDEFPGIPSP |
| | | | | LDAAVECHRGECQAEGVLFFQGDREWF |
| | | | | WDLATGTMKERSWPAVGNCSSALRWLG |
| | | | | RYYCFQGNQFLRFDPVRGEVPPRYPRDV |
| | | | | RDYFMPCPGRGHGHRNGTGHGNSTHHGP |
| | | | | EYMRCSPHLVLSALTSDNHGATYAFSGT |
| | | | | HYWRLDTSRDGWHSWPIAHQWPQGPSA |
| | | | | VDAAFSWEEKLYLVQGTQVYVFLTKGGY |
| | | | | TLVSGYPKRLEKEVGTPHGIILDSVDAAFI |
| | | | | CPGSSRLHIMAGRRLWWLDLKSGAQAT |
| | | | | WTELPWPHEKVDGALCMEKSLGPNSCSA |
| | | | | NGPGLYLIHGPNLYCYSDVEKLNAAKALP |
| | | | | QPQNVTSLLGCTH |
| 34 | HRG | Histidine-rich | P04196 | MKALIAALLLITLQYSCAVSPTDCSAVEPE |
| | | glycoprotein | | AEKALDLINKRRRDGYLFQLLRIADAHLD |
| | | | | RVENTTVYYLVLDVQESDCSVLSRKYWN |
| | | | | DCEPPDSRRPSEIVIGQCKVIATRHSHESQ |
| | | | | DLRVIDFNCTTSSVSSALANTKDSPVLIDF |
| | | | | FEDTERYRKQANKALEKYKEENDDFASF |
| | | | | RVDRIERVARVRGGEGTGYFVDFSVRNCP |
| | | | | RHHFPRHPNVFGFCRADLFYDVEALDLES |
| | | | | PKNLVINCEVFDPQEHENINGVPPHLGHPF |
| | | | | HWGGHERSSTTKPPFKPHGSRDHHHPHK |
| | | | | PHEHGPPPPPDERDHSHGPPLPQGPPPLLP |
| | | | | MSCSSCQHATFGTNGAQRHSHNNNSSDL |
| | | | | HPHKHHSHEQHPHGHHPHAHHPHEHDTH |
| | | | | RQHPHGHHPHGHHPHGHHPHGHHPHGH |
| | | | | HPHCHDFQDYGPCDPPPHNQGHCCHGHG |
| | | | | PPPGHLRRRGPGKGPRPFHCRQIGSVYRL |
| | | | | PPLRKGEVLPLPEANFPSFPLPHHKHPLKP |
| | | | | DNQPFPQSVSESCPGKFKSGFPQVSMFFT |
| | | | | HTFPK |
| 35 | IC1 | Plasma protease | P05155 | MASRLTLLTLLLLLLAGDRASSNPNATSS |
| | | C1 inhibitor | | SSQDPESLQDRGEGKVATTVISKMLFVEPI |
| | | | | LEVSSLPTTNSTTNSATKITANTTDEPTTQ |
| | | | | PTTEPTTQPTIQPTQPTTQLPTDSPTQPTTG |
| | | | | SFCPGPVTLCSDLESHSTEAVLGDALVDES |
| | | | | LKLYHAFSAMKKVETNMAFSPFSIASLLT |
| | | | | QVLLGAGENTKTNLESILSYPKDFTCVHQ |
| | | | | ALKGFTTKGVTSVSQIFHSPDLAIRDTFVN |
| | | | | ASRTLYSSSPRVLSNNSDANLELINTWVA |
| | | | | KNTNNKISRLLDSLPSDTRLVLLNAIYLSA |
| | | | | KWKTTFDPKKTRMEPFHFKNSVIKVPMM |
| | | | | NSKKYPVAHFIDQTLKAKVGQLQLSHNLS |
| | | | | LVILVPQNLKHRLEDMEQALSPSVFKAIM |
| | | | | EKLEMSKFQPTLLTLPRIKVTTSQDMLSIM |
| | | | | EKLEFFDFSYDLNLCGLTEDPDLQVSAMQ |
| | | | | HQTVLELTETGVEAAAASAISVARTLLVF |
| | | | | EVQQPFLFVLWDQQHKFPVFMGRVYDPR |
| | | | | A |
| 5 | IGA1 | Immunoglobulin | P01876 | ASPTSPKVFPLSLCSTQPDGNVVIACLVQG |
| | | heavy constant | | FFPQEPLSVTWSESGQGVTARNFPPSQDA |
| | | alpha 1 | | SGDLYTTSSQLTLPATQCLAGKSVTCHVK |
| | | | | HYTNPSQDVTVPCPVPSTPPTPSPSTPPTPS |
| | | | | PSCCHPRLSLHRPALEDLLLGSEANLTCTL |
| | | | | TGLRDASGVTFTWTPSSGKSAVQGPPERD |
| | | | | LCGCYSVSSVLPGCAEPWNHGKTFTCTA |
| | | | | AYPESKTPLTATLSKSGNTFRPEVHLLPPP |
| | | | | SEELALNELVTLTCLARGFSPKDVLVRWL |
| | | | | QGSQELPREKYLTWASRQEPSQGTTTFAV |
| | | | | TSILRVAAEDWKKGDTFSCMVGHEALPL |
| | | | | AFTQKTIDRLAGKPTHVNVSVVMAEVDG |
| | | | | TCY |
| 6 | IGA2 | Immunoglobulin | P01877 | ASPTSPKVFPLSLDSTPQDGNVVVACLVQ |
| | | heavy constant | | GFFPQEPLSVTWSESGQNVTARNFPPSQD |
| | | alpha 2 | | ASGDLYTTSSQLTLPATQCPDGKSVTCHV |
| | | | | KHYTNSSQDVTVPCRVPPPPPCCHPRLSL |
| | | | | HRPALEDLLLGSEANLTCTLTGLRDASGA |
| | | | | TFTWTPSSGKSAVQGPPERDLCGCYSVSS |
| | | | | VLPGCAQPWNHGETFTCTAAHPELKTPLT |
| | | | | ANITKSGNTFRPEVHLLPPPSEELALNELV |
| | | | | TLTCLARGFSPKDVLVRWLQGSQELPREK |
| | | | | YLTWASRQEPSQGTTTYAVTSILRVAAED |
| | | | | WKKGETFSCMVGHEALPLAFTQKTIDRM |
| | | | | AGKPTHINVSVVMAEADGTCY |
| 36 | IGG1 | Immunoglobulin | P01857 | ASTKGPSVFPLAPSSKSTSGGTAALGCLV |
| | | heavy constant | | KDYFPEPVTVSWNSGALTSGVHTFPAVLQ |
| | | gamma 1 | | SSGLYSLSSVVTVPSSSLGTQTYICNVNHK |
| | | | | PSNTKVDKKVEPKSCDKTHTCPPCPAPEL |
| | | | | LGGPSVFLFPPKPKDTLMISRTPEVTCVVV |
| | | | | DVSHEDPEVKFNWYVDGVEVHNAKTKP |
| | | | | REEQYNSTYRVVSVLTVLHQDWLNGKEY |
| | | | | KCKVSNKALPAPIEKTISKAKGQPREPQV |
| | | | | YTLPPSRDELTKNQVSLTCLVKGFYPSDIA |
| | | | | VEWESNGQPENNYKTTPPVLDSDGSFFLY |
| | | | | SKLTVDKSRWQQGNVFSCSVMHEALHN |
| | | | | HYTQKSLSLSPGK |
| 37 | IGG2 | Immunoglobulin | P01859 | ASTKGPSVFPLAPCSRSTSESTAALGCLVK |
| | | heavy constant | | DYFPEPVTVSWNSGALTSGVHTFPAVLQS |
| | | gamma 2 | | SGLYSLSSVVTVPSSNFGTQTYTCNVDHK |
| | | | | PSNTKVDKTVERKCCVECPPCPAPPVAGP |
| | | | | SVFLFPPKPKDTLMISRTPEVTCVVVDVS |
| | | | | HEDPEVQFNWYVDGVEVHNAKTKPREE |
| | | | | QFNSTFRVVSVLTVVHQDWLNGKEYKCK |
| | | | | VSNKGLPAPIEKTISKTKGQPREPQVYTLP |
| | | | | PSREEMTKNQVSLTCLVKGFYPSDISVEW |
| | | | | ESNGQPENNYKTTPPMLDSDGSFFLYSKL |
| | | | | TVDKSRWQQGNVFSCSVMHEALHNHYT |
| | | | | QKSLSLSPGK |
| 38 | KLKB1 | Plasma | P03952 | MILFKQATYFISLFATVSCGCLTQLYENAF |
| | | kallikrein | | FRGGDVASMYTPNAQYCQMRCTFHPRCL |
| | | | | LFSFLPASSINDMEKRFGCFLKDSVTGTLP |
| | | | | KVHRTGAVSGHSLKQCGHQISACHRDIY |
| | | | | KGVDMRGVNFNVSKVSSVEECQKRCTNN |
| | | | | IRCQFFSYATQTFHKAEYRNNCLLKYSPG |
| | | | | GTPTAIKVLSNVESGFSLKPCALSEIGCHM |
| | | | | NIFQHLAFSDVDVARVLTPDAFVCRTICT |
| | | | | YHPNCLFFTFYTNVWKIESQRNVCLLKTS |
| | | | | ESGTPSSSTPQENTISGYSLLTCKRTLPEPC |
| | | | | HSKIYPGVDFGGEELNVTFVKGVNVCQET |
| | | | | CTKMIRCQFFTYSLLPEDCKEEKCKCFLR |
| | | | | LSMDGSPTRIAYGTQGSSGYSLRLCNTGD |
| | | | | NSVCTTKTSTRIVGGTNSSWGEWPWQVS |
| | | | | LQVKLTAQRHLCGGSLIGHQWVLTAAHC |
| | | | | FDGLPLQDVWRIYSGILNLSDITKDTPFSQI |
| | | | | KEIIIHQNYKVSEGNHDIALIKLQAPLNYT |
| | | | | EFQKPICLPSKGDTSTIYTNCWVTGWGFS |
| | | | | KEKGEIQNILQKVNIPLVTNEECQKRYQD |
| | | | | YKITQRMVCAGYKEGGKDACKGDSGGPL |
| | | | | VCKHNGMWRLVGITSWGEGCARREQPG |
| | | | | VYTKVAEYMDWILEKTQSSDGKAQMQSP |
| | | | | A |
| 39 | TRFE | Serotransferrin | P02787 | MRLAVGALLVCAVLGLCLAVPDKTVRW |
| | | | | CAVSEHEATKCQSFRDHMKSVIPSDGPSV |
| | | | | ACVKKASYLDCIRAIAANEADAVTLDAG |
| | | | | LVYDAYLAPNNLKPVVAEFYGSKEDPQT |
| | | | | FYYAVAVVKKDSGFQMNQLRGKKSCHT |
| | | | | GLGRSAGWNIPIGLLYCDLPEPRKPLEKA |
| | | | | VANFFSGSCAPCADGTDFPQLCQLCPGCG |
| | | | | CSTLNQYFGYSGAFKCLKDGAGDVAFVK |
| | | | | HSTIFENLANKADRDQYELLCLDNTRKPV |
| | | | | DEYKDCHLAQVPSHTVVARSMGGKEDLI |
| | | | | WELLNQAQEHFGKDKSKEFQLFSSPHGK |
| | | | | DLLFKDSAHGFLKVPPRMDAKMYLGYEY |
| | | | | VTAIRNLREGTCPEAPTDECKPVKWCALS |
| | | | | HHERLKCDEWSVNSVGKIECVSAETTEDC |
| | | | | IAKIMNGEADAMSLDGGFVYIAGKCGLV |
| | | | | PVLAENYNKSDNCEDTPEAGYFAIAVVK |
| | | | | KSASDLTWDNLKGKKSCHTAVGRTAGW |
| | | | | NIPMGLLYNKINHCRFDEFFSEGCAPGSK |
| | | | | KDSSLCKLCMGSGLNLCEPNNKEGYYGY |
| | | | | TGAFRCLVEKGDVAFVKHQTVPQNTGGK |
| | | | | NPDPWAKNLNEKDYELLCLDGTRKPVEE |
| | | | | YANCHLARAPNHAVVTRKDKEACVHKIL |
| | | | | RQQQHLFGSNVTDCSGNFCLFRSETKDLL |
| | | | | FRDDTVCLAKLHDRNTYEKYLGEEYVKA |
| | | | | VGNLRKCSTSSLLEACTFRRP |
| 40 | VTNC | Vitronectin | P04004 | MAPLRPLLILALLAWVALADQESCKGRC |
| | | | | TEGFNVDKKCQCDELCSYYQSCCTDYTA |
| | | | | ECKPQVTRGDVFTMPEDEYTVYDDGEEK |
| | | | | NNATVHEQVGGPSLTSDLQAQSKGNPEQ |
| | | | | TPVLKPEEEAPAPEVGASKPEGIDSRPETL |
| | | | | HPGRPQPPAEEELCSGKPFDAFTDLKNGS |
| | | | | LFAFRGQYCYELDEKAVRPGYPKLIRDV |
| | | | | WGIEGPIDAAFTRINCQGKTYLFKGSQYW |
| | | | | RFEDGVLDPDYPRNISDGFDGIPDNVDAA |
| | | | | LALPAHSYSGRERVYFFKGKQYWEYQFQ |
| | | | | HQPSQEECEGSSLSAVFEHFAMMQRDSW |
| | | | | EDIFELLFWGRTSAGTRQPQFISRDWHGV |
| | | | | PGQVDAAMAGRIYISGMAPRPSLAKKQR |
| | | | | FRHRNRKGYRSQRGHSRGRNQNSRRPSR |
| | | | | ATWLSLFSSEESNLGANNYDDYRMDWLV |
| | | | | PATCEPIQSVFFFSGDKYYRVNLRTRRVD |
| | | | | TVDPPYPRSIAQYWLGCPAPGHL |

Table 7A identifies and defines the glycan structures included in Table 1A. Table 7A identifies a graphical representation of the structure and a coded representation of the composition for each glycan structure included in Table 1A. As used herein, the 4-digit GL NO. is a designation that represents the number of hexoses, the number of HexNAcs, the number of Fucoses, and the number of Neuraminic Acids.

TABLE 7A
Glycan Structure GL NOS: Structure and Composition

| Glycan | | |
|---|---|---|
| Structure | | |
| GL NO. | Glycan Symbol Structure | Glycan Composition |
| 1102 | 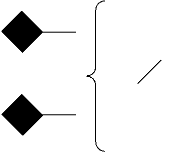 | Hex(1)HexNAc(1) Fuc(0)NeuAc(2) |
| 1300 | 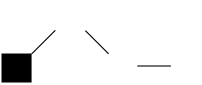 | Hex(1)HexNAc(3) Fuc(0)NeuAc(0) |
| 3500 | 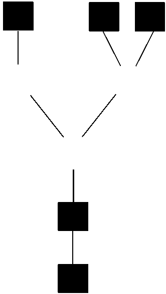 | Hex(3)HexNAc(5) Fuc(0)NeuAc(0) |
| | 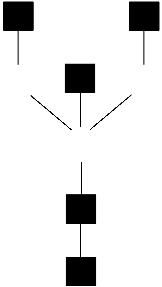 | |
| 4310 | 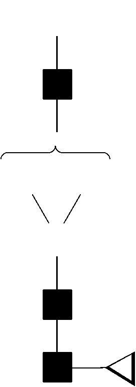 | Hex(4)HexNAc (3)Fuc(1)NeuAc(0) |
| 5400 | 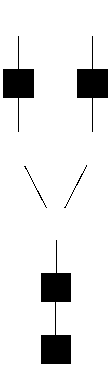 | Hex(5)HexNAc(4) Fuc(0)NeuAc(0) |
| 5401 | 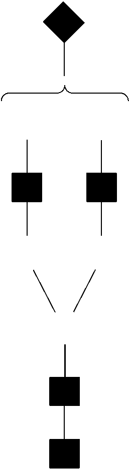 | Hex(5)HexNAc(4) Fuc(0)NeuAc(1) |
| 5402 | 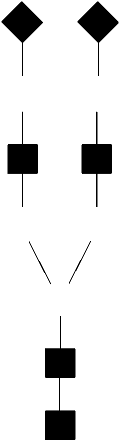 | Hex(5)HexNAc(4) Fuc(0)NeuAc(2) |
| 5412 | 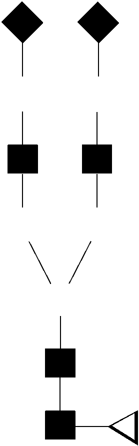 | Hex(5)HexNAc(4) Fuc(1)NeuAc(2) |
| 5421 | 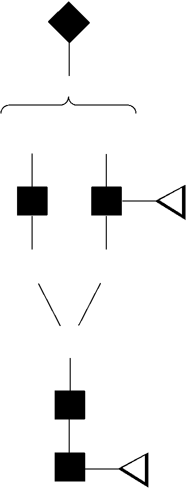 | Hex(5)HexNAc(4) Fuc(2)NeuAc(1) |
| 6503 | 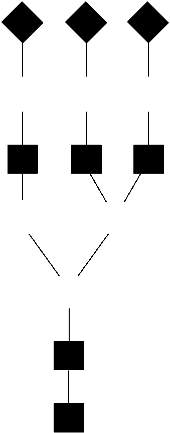 | Hex(6)HexNAc(5) Fuc(0)NeuAc(3) |
| 7604 | 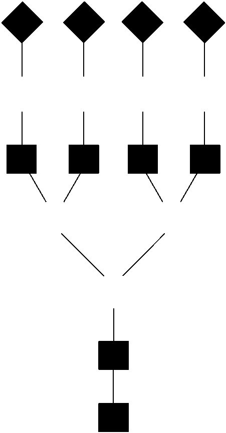 | Hex(7)HexNAc(6) Fuc(0)NeuAc(4) |

Table 7B identifies and defines the glycan structures included in Table 1B. Table 7B identifies a graphical representation of the structure and a coded representation of the composition for each glycan structure included in Table 1B. As used herein, the 4-digit GL NO. is a designation that represents the number of hexoses, the number of HexNAcs, the number of Fucoses, and the number of Neuraminic Acids.

Tables 7A and 7B illustrate the symbol structure and composition of detected glycan moieties that correspond to glycopeptides of Tables 1A and 1B, respectively, based on the Glycan GL NO. The term Symbol Structure illustrates a geometric linking structure of the carbohydrates where the bottommost carbohydrate such as N-acetylglucosamine is bound to the designated amino acid for an N-linked glycan and the rightmost carbohydrate such as N-acetylgalactosamine is bound to the designated amino acid for an O-linked glycan. For reference, N-linked glycans have a glycan attached to the amino acid asparagine and O-linked glycans have a glycan attached to either a serine or a threonine. It should be noted that glycan GL NO 1102 and 1300 in Table 7A represent O-linked glycans. All other glycans in Tables 7A and 7B represent N-linked glycans.

The term Composition refers to the number of various classes of carbohydrates that make up the glycan. The quantity for each class of carbohydrate is depicted as a number in parenthesis to the right of an abbreviation that corresponds to the class of the carbohydrate. The abbreviations for these classes are Hex, HexNAc, Fuc, and NeuAc that respectively correspond to hexose, N-acetylhexosamine, fucose, and N-acetylneuraminic acid. It should be noted that hexose sugars include glucose, galactose, and mannose; and N-acetylhexosamine sugars includes N-acetylglucosamine, N-acetylgalactosamine, and N-acetylmannosamine. In various embodiments, the terms Neu5Ac, NeuAc, and N-acetylneuraminic acid may be referred to as sialic acid.

Referring back to Tables 7A and 7B, for some entries, there are two symbol structures provided for one Glycan Structure GL NO such as, for example, Glycan Structure GL NO 3500. Thus, the identify of a peptide that references a Glycan Structure GL NO that has two symbol structures could be one of two possibilities based on the MRM of the LC-MS analysis. In some instances, a bracket symbol is used as part of the Symbol Structure (e.g., 4310) to indicate that the precise bonding linkage is not exactly known, but that the linking line segment is attached to one of the plurality of adjacent carbohydrates immediately adjacent to the bracket.

TABLE 7B
N-Linked Glycan Structure GL NOS: Structure and Glycan Composition

| Glycan | | |
|---|---|---|
| Structure | | |
| GL NO. | Glycan Symbol Structure | Glycan Composition |
| 6503 | 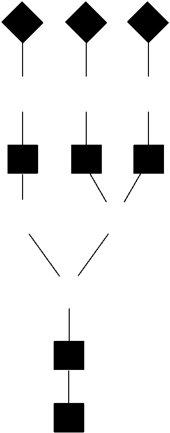 | Hex(6)HexNAc(5) Fuc(0)NeuAc(3) |
| 6513 | 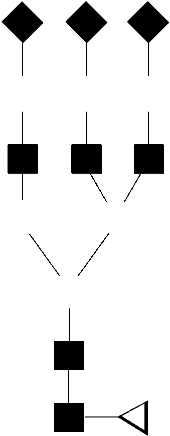 | Hex(6)HexNAc(5) Fuc(1)NeuAc(3) |
| 5411 | 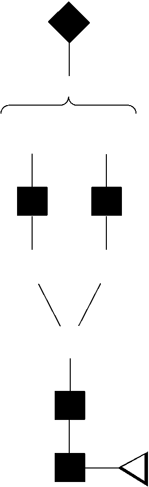 | Hex(5)HexNAc(4) Fuc(1)NeuAc(1) |
| 5200 | 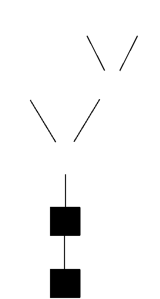 | Hex(5)HexNAc(2) Fuc(0)NeuAc(0) |
| 5401 | 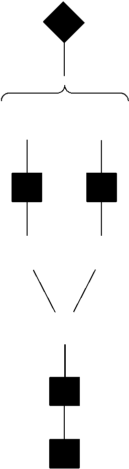 | Hex(5)HexNAc(4) Fuc(0)NeuAc(1) |
| 5402 | 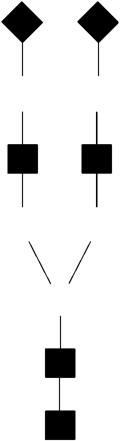 | Hex(5)HexNAc(4) Fuc(0)NeuAc(2) |
| 3500 | 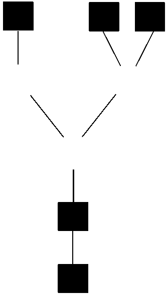 | Hex(3)HexNAc(5) Fuc(0)NeuAc(0) |
| | 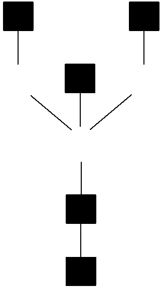 | |
| 3300 | 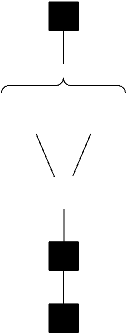 | Hex(3)HexNAc(3) Fuc(0)NeuAc(0) |
| 3410 | 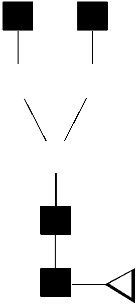 | Hex(3)HexNAc(4) Fuc(1)NeuAc(0) |
| 4400 | 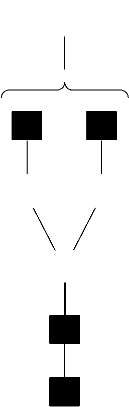 | Hex(4)HexNAc(4) Fuc(0)NeuAc(0) |
| 4410 | 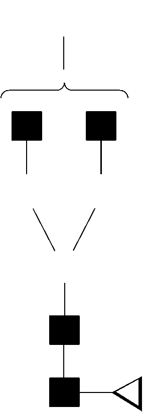 | Hex(4)HexNAc(4) Fuc(1)NeuAc(0) |

Legend for Table 7A and 7B 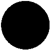
Glc 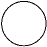
Gal

[symbol]

Man

[symbol]

Fuc

[symbol]

Neu5Ac 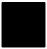
GlcNAc 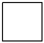
GalNAc

[symbol]

ManNAc

The identity of the various monosaccharides is illustrated by the Legend section located at the end of Tables 7A and 7B. The abbreviations of the Legend are Glc that represents glucose and is indicated by a dark circle, Gal that represents galactose and is indicated by an open circle, Man that represents mannose and is indicated by a circle with intermediate grey shading, Fuc that represents fucose and is indicated by a dark triangle, Neu5Ac that represents N-acetylneuraminic acid and is indicated by a dark diamond, GlcNAc that represents N-acetylglucosamine and is indicated by a dark square, GalNAc that represents N-acetylgalactosamine and is indicated by an open square, and ManNAc that represents N-acetylmannosamine and is indicated by a square with intermediate grey shading.

Aspects of the disclosure include kits comprising one or more compositions, each comprising one or more peptide structures of the disclosure that can be used as assay standards, and instructions for use. Kits in accordance with one or more embodiments described herein may include a label indicating the intended use of the contents of the kit. The term “label” as used herein with respect to a kit includes any writing, or recorded material supplied on or with a kit, or that otherwise accompanies a kit.

The peptide structures and the transitions produced therefrom, as described herein, may be useful for diagnosing and treating an NPC disease state. A transition includes a precursor ion and at least one product ion grouping. As reviewed herein, the peptide structures in Table 1, as well as their corresponding precursor ion and product ion groupings (these ions having defined m/z ratios or m/z ratios that fall within the m/z ranges identified herein), can be used in mass spectrometry-based analyses to diagnose and facilitate treatment of diseases, such as, for example, NPC.

Aspects of the disclosure include methods for analyzing one or more peptide structures, as described herein. In some embodiments, the methods involve processing a sample from a patient to generate a prepared sample that can be inputted into a mass spectrometry system (e.g., a reaction monitoring mass spectrometry system). In certain embodiments, processing the sample can comprise performing one or more of: a denaturation procedure, a reduction procedure, an alkylation procedure, and a digestion procedure. The denaturation and reduction procedures may be implemented in a manner similar to, for example, denaturation and reduction 202 in FIG. 2. The alkylation procedure may be implemented in a manner similar to, for example, alkylation procedure 204 in FIG. 2. The digestion procedure may be implemented in a manner similar to, for example, digestion procedure 206 in FIG. 2.

In some embodiments, the methods for analyzing one or more peptide structures involve detecting a set of product ions generated by a reaction monitoring mass spectrometry system in which one or more product ions may correspond to each of the one or more peptide structures that have been inputted into the mass spectrometry system. As described herein, each peptide structure can be converted into a set of product ions having a defined m/z ratio, as provided in Table 4 or an m/z ratio within an identified m/z ratio as provided in Table 4. In some embodiments, the methods involve generating quantification (e.g., abundance) data for the one or more product ions detected using the reaction monitoring mass spectrometry system.

In some embodiments, the methods further comprise generating a diagnosis output using the quantification data and a model that has been trained using supervised or unsupervised machine learning. In certain embodiments, the reaction monitoring mass spectrometry system may include multiple/selected reaction monitoring mass spectrometry (MRM/SRM-MS) to detect the one or more product ions and generate the quantification data.

### IX. Representative Experimental Results

### IX.A. Sample Preparation and Mass Spectrometry Data Production

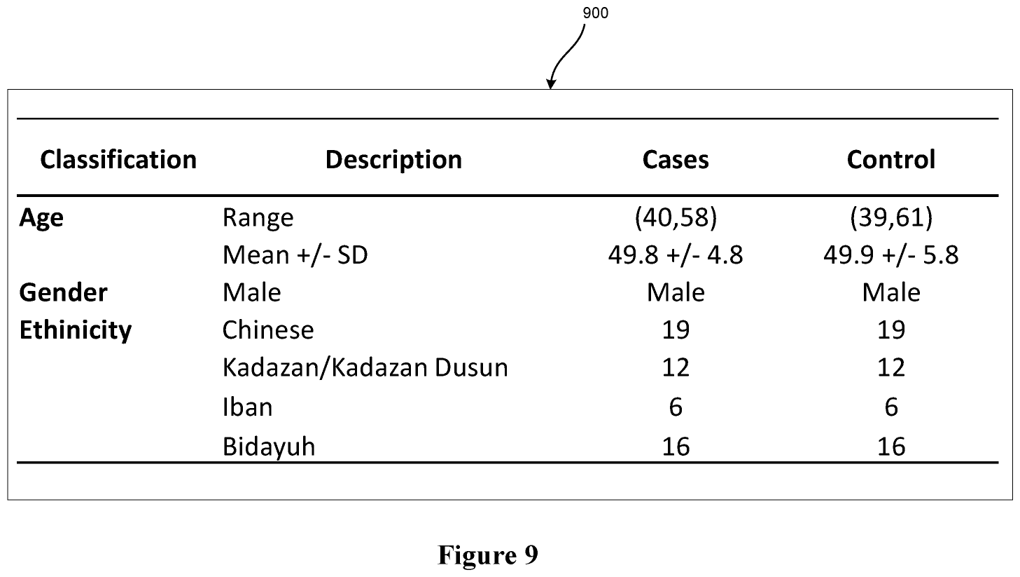

FIG. 9 is a table of the sample population used for the experiments in accordance with one or more embodiments. As table 900 illustrates, the sample population included serum samples from 106 patients with a biopsy-proven diagnosis of NPC (53 males) and healthy (53 males). NPC cases comprise 19 Chinese males, 12 Kadazan/Kadazan Dusun males, 6 Than males, and 16 Bidayuh males ranging in age from 40-58 years old including a mean age of 49.8 years with a standard deviation of 4.8. Control cases comprise 19 Chinese males, 12 Kadazan/Kadazan Dusun males, 6 Than males, and 16 Bidayuh males including a mean age of 49.9 years with a standard deviation of 5.8.

For data analysis, 5 samples (for 5 patients) were excluded such that the data analysis was performed using 101 samples of the sample population.

Prior to analysis, serum samples were reduced with DTT and alkylated with IAA followed by digestion with trypsin in a water bath at 37° C. for 18 hours. To quench the digestion, formic acid was added to each sample after incubation to a final concentration of 1%.

Digested serum samples were injected into a triple quadrupole mass spectrometer (MS). Separation of the peptide structures (glycosylated and aglycosylated) was performed using a 48-min binary gradient. The triple quadrupole MS was operated in dynamic multiple reaction monitoring (dMRM) mode. Samples were injected in a randomized fashion with regard to underlying phenotype, and reference pooled serum digests were injected interspersed with study samples, at every 10^{th} sample position throughout the run.

An MRM analysis was performed on the peptide structures, representing a total of 62 high-abundance serum glycoproteins. A transition list consisted of glycopeptide structures as well as of aglycosylated peptide structures from each glycoprotein. The python library Scikit-learn (https://scikit-learn.org/stable/) was used for statistical analyses and for building machine learning models.

Normalized abundance data was generated for the peptide structures using the following formula:

$$Normalized abundance = ( raw abundance of a peptide structure in sample \text{} / raw abundance of the corresponding aglycosylated peptide from the same glycoprotein ) \text{} / average relative abundance of the same glycopeptides or peptides in the flanking pooled reference serum samples$$

Relative abundance was calculated as the ratio of the raw abundance of any given glycopeptide to the sum of raw abundances of all glycopeptides.

### IX.B. Identification of Peptide Structures and Machine Learning Model

A panel consisting of 409 glycopeptides across 66 proteins and 101 individuals were assessed. To differentiate NPC cases from control, differential expression analysis was performed. Linear regression was performed on a marker-by-marker basis with FDR<0.01.

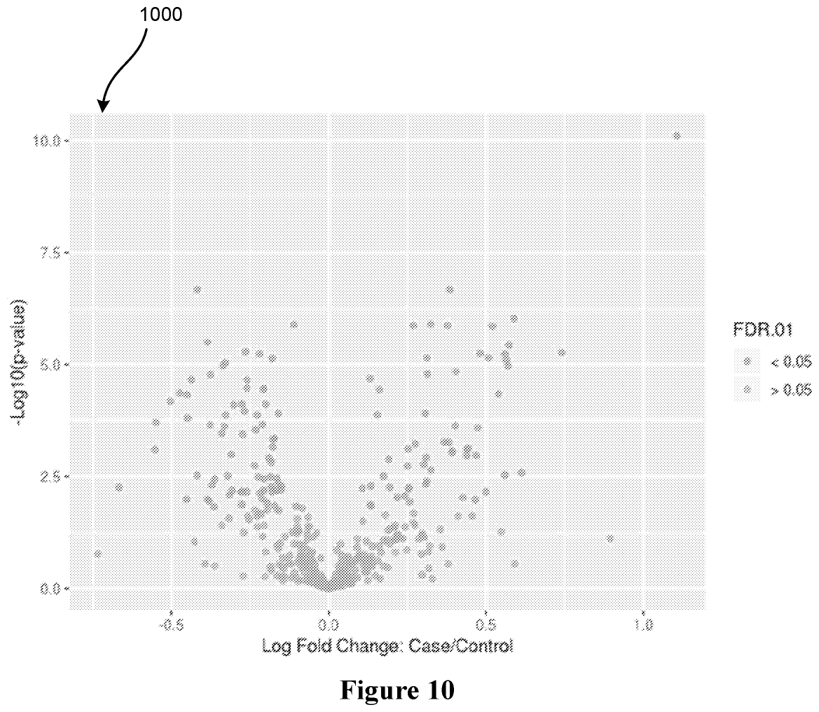

FIG. 10 is an illustration of a volcano plot 1000 highlighting differentially expressed peptides between NPC cases and control in accordance with one or embodiments. 62 glycopeptides were identified to be differentially expressed.

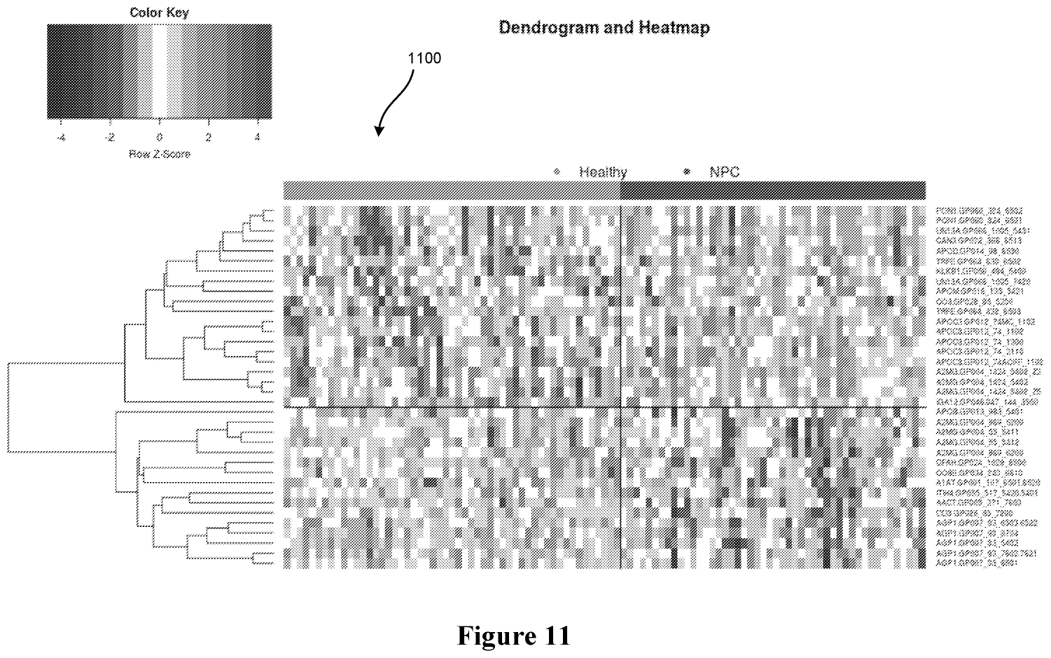

FIG. 11 is an illustration of a heat map 1100 depicting the portion of the 62 peptide structures that were significantly differentially expressed between the healthy/control and NPC cancer patients in accordance with one or embodiments. This portion includes those peptide structures (36 peptide structures) having a false discovery rate (FDR) of less than 0.001. The Y-axis represents the individual targeted glycopeptides in the MS spectra and the X-axis represents the individual patients in the cancer and non-cancer groups. The spectral shading in the matrix represents the ratio level of glycopeptide peaks in the individual compared to the average level in the non-cancer group. The color code denotes z-scaled values of peptides signal intensities.

A supervised machine learning model (e.g., regression model) was then trained using quantification data for 80 of the subjects. LASSO regression identified 19 peptide structures with non-zero coefficients as shown in Table 1 in order of most significant to least significant with respect to the disease indicator generated by the machine learning model.

### IX.C. Validation and Results

The subject quantification data not used to train the model was used to validate the model.

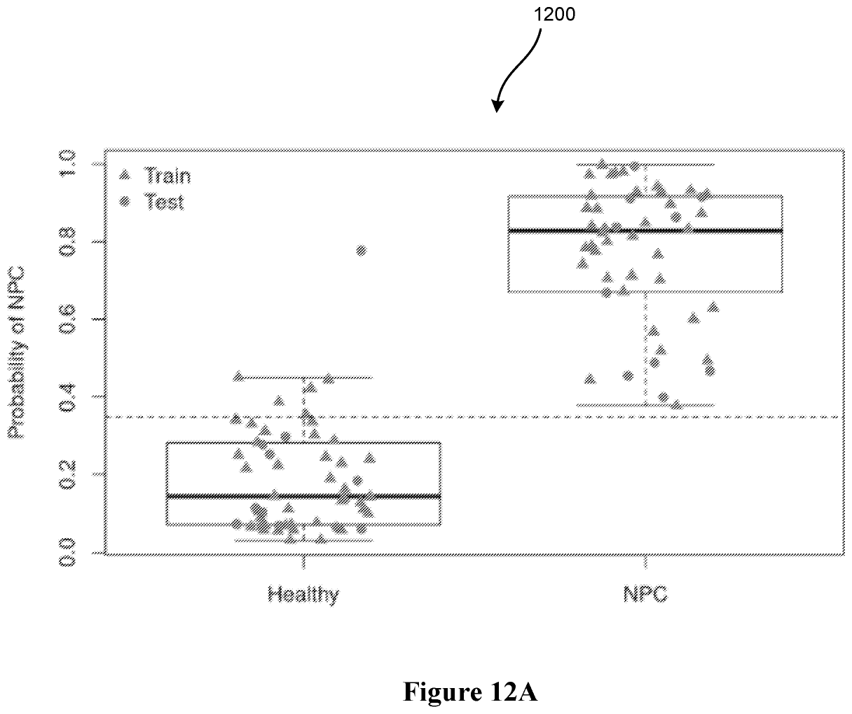

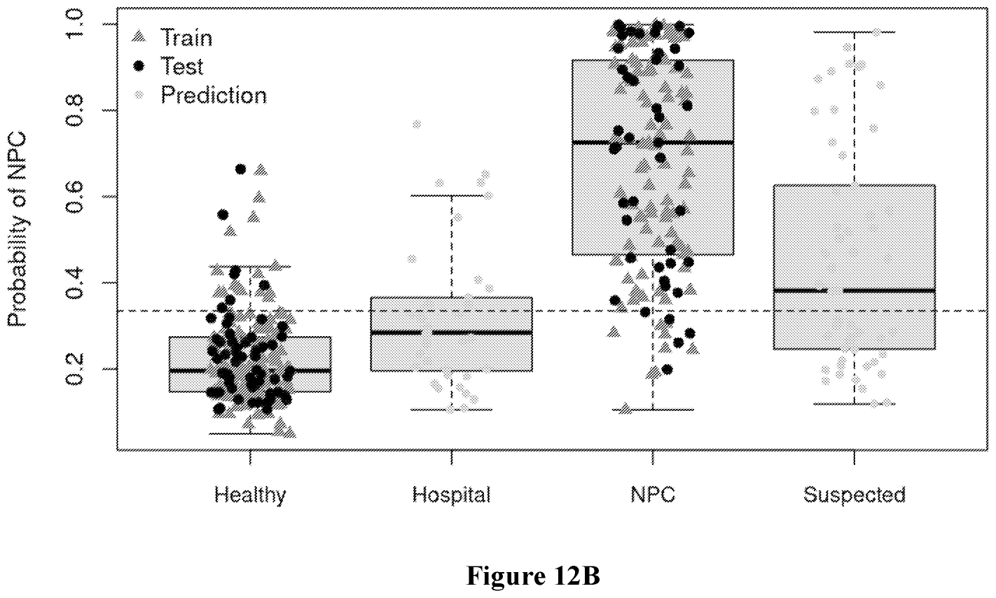

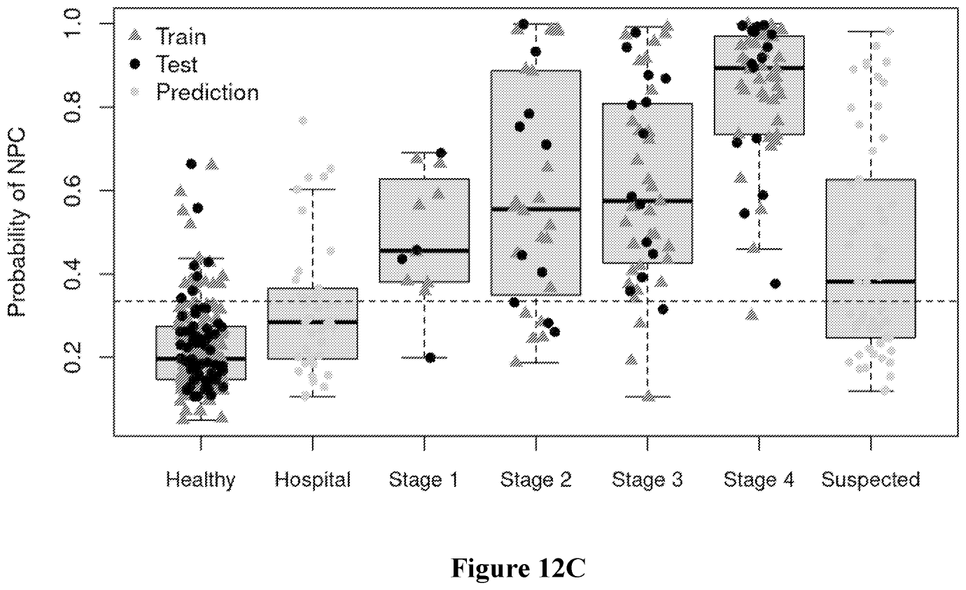

FIG. 12 is a plot diagram 1200 illustrating validation of the disease indicator's ability to determine whether a biological sample evidences the NPC disease state or a non-NPC state (e.g., healthy, control, etc.) in accordance with one or embodiments. As depicted on the left-hand portion of the chart, a disease indicator of about 0.0 to about 0.05 was generally accurate in classifying as a non-NPC state (e.g., NPC negative). As depicted on the right-hand portion of the chart, a disease indicator of about 0.5 to about 1.00 was generally accurate in classifying as the NPC disease state (e.g., NPC positive).

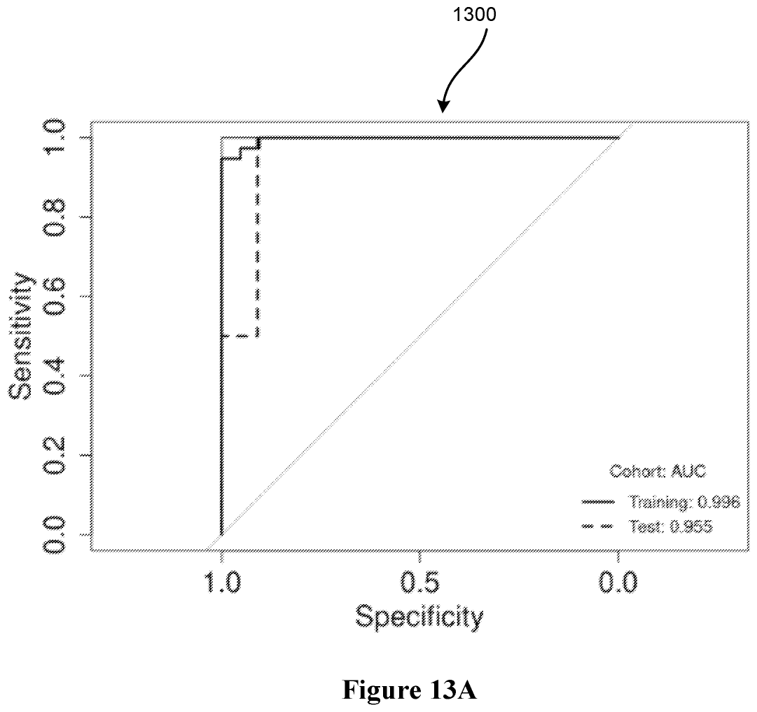

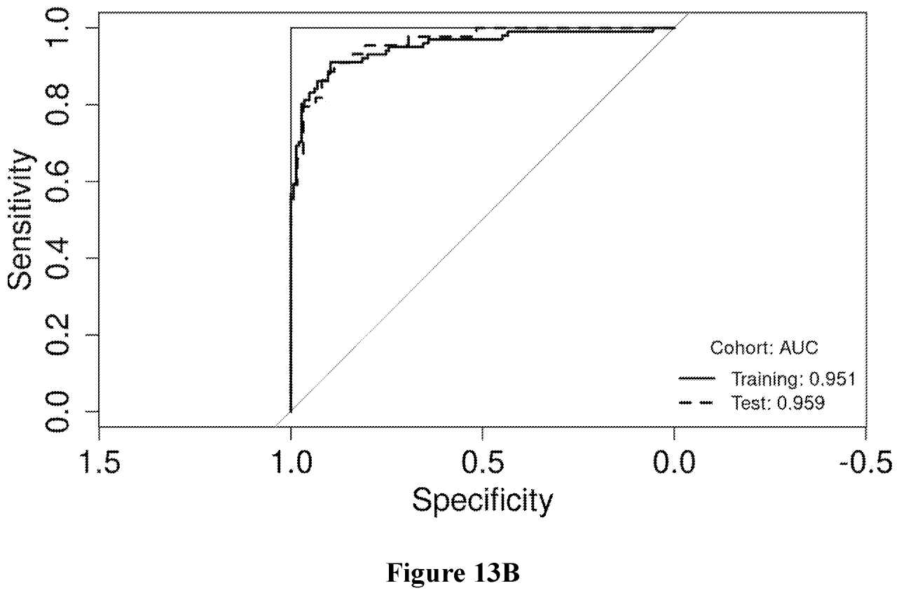

FIG. 13 is an illustration of a receiver operating characteristic (ROC) curve 1300 in accordance with one or embodiments. The area under curve (AUC) was calculated to be 0.996 for the training data and 0.955 for the test data.

### IX.D. Sample Preparation and Mass Spectrometry Data Production

Table 8 shows the various classification of the sample population (N=442 subjects) used for the experiments in accordance with one or more embodiments. Table 8 illustrates, the sample population included serum samples from 145 subjects with a biopsy-proven diagnosis of NPC, 207 subjects that were healthy controls not having NPC, 37 subjects that were classified as “Hospital”, and 53 subjects that were classified as “Suspected.” The subjects classified as “Hospital” were patients visiting a hospital for having a symptom or disease that was not cancer related and were not visiting the ear-nose-throat (ENT) clinic. The “Hospital” samples can be used as a verification of the NPC model algorithm that was expected to provide results similar to the Healthy controls. The subjects classified as “Suspected” were patients visiting an ENT clinic for having a symptom of NPC but the histopathology review of the biopsy tissue sample did not indicate NPC. The “Suspected” sample can be tested with the NPC model algorithm to see if it can provide insight to detecting a pre-cancer stage of NPC.

TABLE 8

| | | Number of NPC |
|---|---|---|
| | Overall, | Subjects based on |
| Classification | N = 442 | Stage |
| Healthy (control) | 207 | |
| NPC | 145 | |
| NPC Stage 1 | | 12 |
| NPC Stage 2 | | 32 |
| NPC Stage 3 | | 47 |
| NPC Stage 4 | | 54 |
| Hospital | 37 | |
| Suspected | 53 | |
| Gender | | |
| Female | 95 | |
| Male | 347 | |
| Age | 47.1 years mean | |
| | (13.6 SD) | |

Prior to analysis, serum samples were reduced with DTT and alkylated with IAA followed by digestion with trypsin in a water bath at 37° C. for 18 hours. To quench the digestion, formic acid was added to each sample after incubation to a final concentration of 1%.

Digested serum samples were injected into a triple quadrupole mass spectrometer (MS). Separation of the peptide structures (glycosylated and aglycosylated) was performed using a 70-min binary gradient. The triple quadrupole MS was operated in dynamic multiple reaction monitoring (dMRM) mode. Samples were injected in a randomized fashion with regard to underlying phenotype, and reference pooled serum digests were injected interspersed with study samples, at every 10th sample position throughout the run.

An MRM analysis was performed on the peptide structures, representing a total of 78 high-abundance serum glycoproteins. A transition list consisted of glycopeptide structures as well as of aglycosylated peptide structures from each glycoprotein. The R programming language was used for statistical analyses and for building machine learning models.

Normalized abundance data was generated for the peptide structures by using the following formula:

$$Normalized abundance = ( raw abundance of a peptide structure in sample \text{} / raw abundance of the corresponding aglycosylated peptide from the same glycoprotein ) \text{} / average relative abundance of the same glycopeptides or peptides in the flanking pooled reference serum samples$$

Relative abundance was calculated as the ratio of the raw abundance of any given glycopeptide to the sum of raw abundances of all glycopeptides.

Glycopeptide concentration was calculated by multiplying relative abundance value by a ratio based on the concentration and the relevant abundance measured for an internal peptide standard.

### IX.E. Identification of Peptide Structures and Machine Learning Model

A panel consisting of 521 glycopeptides and 16 non-glycosylated peptides across 78 proteins and 352 individuals (healthy and NPC) were assessed. To differentiate NPC cases from control, differential expression analysis was performed for the glycopeptide and peptide (markers) concentrations. Multiple comparison adjustment of the raw p-values was performed using the Benjamini-Hochberg False Discovery Rate (FDR) procedure. Markers with a FDR-adjusted p-value <0.01 were selected. In total, there were 351 markers that were found to be differentially expressed between healthy and NPC or healthy and suspected with a FDR-adjusted p-value <0.01.

A supervised machine learning model (e.g., regression model) was then trained using the concentration data of the 351 markers for the healthy and NPC subjects. LASSO regression identified 18 glycopeptide structures with non-zero coefficients as shown in Table 2B.

### IX.F. Validation and Results

The subject quantification data not used to train the model was used to validate the model. In various embodiments, 70% of the 352 subjects were selected to be part of the training data set and 30% of the 352 subjects were selected to be part of the test data set.

FIG. 12B is a plot diagram showing the probability distributions for the various groups using the multivariable model for predicting the NPC state vs. Healthy controls in accordance with one or more embodiments. The probability output of the model was shown for both the training data set (triangles) and the test data set (dark circles). Referring back to FIG. 12B, a disease indicator or probability value about 0.0 to about 0.33 was generally accurate in classifying as a non-NPC state (e.g., NPC negative or Healthy). A disease indicator or probability value of about 0.33 to about 1.00 was generally accurate in classifying as the NPC disease state (e.g., NPC positive). The “Hospital” data set (37 subjects) was tested with the model and outputted a probability profile that was similar to the Healthy cohort. The “Suspected” data set (53 subjects) was tested with the model and outputted a probability profile that bridged between the Healthy cohort and the NPC cohort, which suggests that the model may predict whether some of the subjects are at a higher risk of developing NPC in the future.

FIG. 12C is a plot diagram of the probability distributions for healthy, hospital, stage 1 NPC, stage 2 NPC, stage 3 NPC, stage 4 NPC, and suspected samples. The probability output of the model shows an approximate increase from Stage 1 to Stage 4.

FIG. 13B is an illustration of a receiver operating characteristic (ROC) curve in accordance with the model using testing and training data with the 352 subjects. The area under curve (AUC) was calculated to be 0.951 for the training data and 0.959 for the test data.

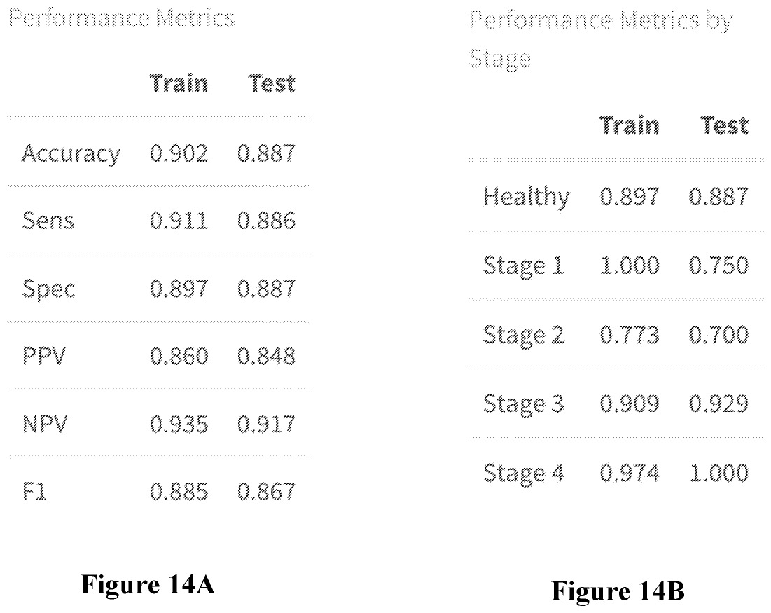

FIG. 14A shows the model performance metrics of accuracy, sensitivity, specificity, positive predictive value (PPV), negative predictive value (NPV), and harmonic mean of precision and sensitivity (F1) for the training data set and test set based on 246 subjects and 106 subjects, respectively. FIG. 14B shows the model performance metrics of accuracy by stage for the training data set and test set based on 246 subjects and 106 subjects, respectively.

### X. Recitation of Embodiments

1. A method for diagnosing a subject with respect to a nasopharyngeal carcinoma (NPC) disease state, the method comprising:
receiving peptide structure data corresponding to a biological sample obtained from the subject;
analyzing the peptide structure data using a supervised machine learning model to generate a disease indicator that indicates whether biological sample evidences the NPC disease state based on at least 3 peptide structures selected from a group of peptide structures identified in Table 1A and/or Table 1B,
   wherein the group of peptide structures in Table 1A and/or Table 1B is associated with the NPC disease state; and
   wherein the group of peptide structures is listed in Table 1A and/or Table 1B with respect to relative significance to the disease indicator; and
generating a diagnosis output based on the disease indicator.

2. The method of embodiment 1, wherein the disease indicator comprises a score.

3. The method of embodiment 2, wherein generating the diagnosis output comprises:
determining that the score falls above a selected threshold; and
generating the diagnosis output based on the score falling above the selected threshold, wherein the diagnosis output includes a positive diagnosis for the NPC state.

4. The method of embodiment 2, wherein generating the diagnosis output comprises:
determining that the score falls below a selected threshold; and
generating the diagnosis output based on the score falling below the selected threshold, wherein the diagnosis output includes a negative diagnosis for the NPC state.

5. The method of any one of embodiments 1-4, wherein analyzing the peptide structure data comprises:
analyzing the peptide structure data using a regression model.

6. The method of any one of embodiments 1-5, wherein the at least one peptide structure comprises a glycopeptide structure defined by a peptide sequence and a glycan structure linked to the peptide sequence at a linking site of the peptide sequence, as identified in Table 1A, with the peptide sequence being one of SEQ ID NOS: 14-28 as defined in Table 1A and/or as identified in Table 1B, with the peptide sequence being one of SEQ ID NOS: 15, 20, or 41-53.

7. The method of any one of embodiments 1-6, further comprising:
training the supervised machine learning model using training data,
wherein the training data comprises a plurality of peptide structure profiles for a plurality of subjects and a plurality of diagnoses for the plurality of subjects.

8. The method of embodiment 7, further comprising:
performing a differential expression analysis using initial training data to compare a first portion of the plurality of subjects diagnosed with the NPC disease state versus a second portion of the plurality of subjects diagnosed with a non-NPC state; and
identifying a training group of peptide structures based on the differential expression analysis for use as prognostic markers for the NPC disease state; and
forming the training data based on the training group of peptide structures identified.

9. The method of embodiment 8, wherein the non-NPC state includes at least one of a healthy state or a control state.

10. The method of embodiment 7, wherein training the supervised machine learning model using the training data includes reducing the training group of peptide structures to the group of peptide structures identified in Table 1A and/or Table 1B.

11. The method of any one of embodiments 1-10, wherein the supervised machine learning model comprises a logistic regression model.

12. The method of any one of embodiments 1-11, wherein the peptide structure data comprises quantification data for a peptide structure of the set of peptide structures, wherein the quantification data comprises at least one of an abundance, a relative abundance, a normalized abundance, a relative quantity, an adjusted quantity, a normalized quantity, a relative concentration, an adjusted concentration, or a normalized concentration.

13. The method of any one of embodiments 1-12, wherein the peptide structure data is generated using multiple reaction monitoring mass spectrometry (MRM-MS).

14. The method of any one of embodiments 1-13, further comprising:
creating a sample from the biological sample; and
preparing the sample using reduction, alkylation, and enzymatic digestion to form a prepared sample that includes a set of peptide structures.

15. The method of embodiment 14, further comprising:
generating the peptide structure data from the prepared sample using multiple reaction monitoring mass spectrometry (MRM-MS).

16. The method of any one of embodiments 1-15, wherein generating the diagnosis output comprises:
generating a report that identifies that the biological sample evidences the NPC disease state.

17. The method of any one of embodiments 1-16, further comprising:
generating a treatment output based on at least one of the diagnosis output or the disease indicator.

18. The method of embodiment 17, wherein the treatment output comprises at least one of an identification of a treatment to treat the subject and a treatment schedule.

19. The method of embodiment 18, wherein the treatment comprises at least one of radiation therapy, chemoradiotherapy, surgery, or a targeted drug therapy.

20. The method of embodiment 19, wherein the targeted drug therapy comprises cetuximab.

21. A method of training a model to diagnose a subject with respect to a nasopharyngeal carcinoma (NPC) disease state, the method comprising:
receiving quantification data for a panel of peptide structures for a plurality of subjects,
   wherein the plurality of subjects includes a first portion diagnosed with the NPC disease state and a second portion diagnosed with a non-NPC state;
   wherein the quantification data comprises a plurality of peptide structure profiles for the plurality of subjects; and
training a machine learning model using the quantification data to diagnose a biological sample with respect to the NPC disease state using a group of peptide structures associated with the NPC disease state,
   wherein the group of peptide structures is identified in Table 1A and/or Table 1B; and
   wherein the group of peptide structures is listed in Table 1A and/or Table 1B with respect to relative significance to making the diagnosis.

22. The method of embodiment 21, wherein the machine learning model comprises a logistic regression model.

23. The method of embodiment 22, wherein the logistic regression model comprises a LASSO regression model.

24. The method of any one of embodiments 21-23, wherein training the machine learning model comprises:
training the machine learning using a portion of the quantification data corresponding to a training group of peptide structures that is a subset of the plurality of peptide structures.

25. The method of embodiment 24, further comprising:
performing a differential expression analysis using the quantification data for the plurality of subjects.

26. The method of embodiment 25, further comprising:
identifying the training group of peptide structures based on the differential expression analysis, wherein the training group of peptide is a subset of the plurality of peptide structures relevant to diagnosing the NPC disease state.

27. The method of embodiment 21, wherein the non-NPC state includes at least one of a healthy state or a control state.

28. The method of embodiment 21, wherein training the machine learning model includes reducing the training group of peptide structures to the group of peptide structures identified in Table 1A and/or Table 1B.

29. The method of any one of embodiments 21-28, wherein the quantification data for the panel of peptide structures for the plurality of subjects diagnosed with the plurality of NPC disease states comprises at least one of an abundance, a relative abundance, a normalized abundance, a relative quantity, an adjusted quantity, a normalized quantity, a relative concentration, an adjusted concentration, or a normalized concentration.

30. A method of treating nasopharyngeal carcinoma (NPC) in a patient, comprising:
receiving a biological sample from a patient;
determining a quantity of each peptide structure identified in Table 1A and/or Table 1B in the biological sample using MRM-MS;
analyzing the quantity of each peptide structure using a machine learning model to generate a disease indicator;
generating a diagnosis output based on the disease indicator that classifies the biological sample as evidencing an NPC disease state;
administering a treatment to the patient based on at least one of the diagnosis output or the disease indicator.

31. The method of embodiment 30, wherein administering the treatment comprises:
administering a radiation treatment to the patient via intravenous or oral administration, surgery, chemotherapy, targeted drug therapy, and/or immunotherapy.

32. The method of embodiment 30 or 31, wherein administering the treatment comprises
administering Cetuximab, Cisplatin, 5-fluorouracil (5-FU), gemcitabine, carboplatin, Epirubicin, Paclitaxel, Docetaxel, Gemcitabine, Capecitabine, Methotrexate, Pembrolizumab, Nivolumab, or a combination thereof.

33. The method of embodiment 32, wherein Cetuximab is administered at 400 mg/m2 loading dose and then 250 mg/m2/w 8 cycles.

34. The method of embodiments 32 or 33, wherein Cisplatin is administered at 100 mg/m2 intravenously on day 1 plus 5-FU 1000 mg/m2/day by continuous IV infusion on days 1-4 every 3wk.

35. The method of embodiment 32 or 33, wherein Cisplatin is administered intravenously at the fixed dose of 30 mg/m2 on days 1-3 and Gemcitabine is intravenously administered over 30 min infusion with the dose escalated from 800 to 1200 mg/m2 on days 1 and 8.

36. The method of any one of embodiments 32-35, wherein carboplatin is administered at 6 mg ml/min as an intravenous bolus, followed by paclitaxel at 135 mg/ml2 given as an i.v. infusion over 3 h.

37. A method of monitoring a subject for a nasopharyngeal carcinoma (NPC), the method comprising:
receiving peptide structure data of a first biological sample obtained from a subject at a first timepoint;
analyzing the peptide structure data of the first biological sample using a supervised machine learning model to generate a first disease indicator based on at least 3 peptide structures selected from a group of peptide structures identified in Table 1A and/or Table 1B,
   wherein the group of peptide structures in Table 1A and/or Table 1B comprises a group of
   peptide structures associated with the NPC disease state;
receiving peptide structure data of a second biological sample obtained from the subject at a second timepoint;
analyzing the peptide structure data of the second biological sample using the supervised machine learning model to generate a second disease indicator based on the at least 3 peptide structures selected from the group of peptide structures identified in Table 1A and/or Table 1B; and
generating a diagnosis output based on the first disease indicator and the second disease indicator.

38. The method of embodiment 37, wherein generating the diagnosis output comprises: comparing the second disease indicator to the first disease indicator.

39. The method of embodiment 37 or embodiment 38, wherein the first disease indicator indicates that the first biological sample evidences the NPC disease state and the second disease indicator indicates that the second biological sample does not evidence the NPC disease state.

40. The method of any one of embodiments 37-39, wherein the diagnosis output indicates treatment effectiveness.

41. The method of any one of embodiments 37-40, wherein the supervised machine learning model is a logistic regression model.

42. The method of any one of embodiments 37-41, further comprising treating the subject after the first biological sample is obtained and before the second biological sample is obtained.

43. A composition comprising at least one of peptide structures PS-1 to PS-19 identified in Table 1A and/or at least one of peptide structures PS-3, PS-8, PS-9, or PS-20 to PS-35 identified in Table 1B.

44. A composition comprising a peptide structure or a product ion, wherein:
the peptide structure or product ion comprises an amino acid sequence having at least 90% sequence identity to any one of SEQ ID NOS: 14-28, corresponding to peptide structures PS-1 to PS-19 in Table 1A; and
the product ion is selected as one from a group consisting of product ions identified in Table 4A including product ions falling within an identified m/z range; and/or
90% sequence identity to any one of SEQ ID NOS: 15, 20, or 41-53, corresponding to peptide structures PS-3, PS-8, PS-9, or PS-20 to PS-35 identified in Table 1B; and
the product ion is selected as one from a group consisting of product ions identified in Table 4B including product ions falling within an identified m/z range.

45. A composition comprising a glycopeptide structure selected as one from a group consisting of peptide structures PS-1 to PS-19 identified in Table 1A, wherein:
the glycopeptide structure comprises:
   an amino acid peptide sequence identified in Table 5A as corresponding to the glycopeptide structure; and
   a glycan structure identified in Table 7A as corresponding to the glycopeptide structure in which the glycan structure is linked to a residue of the amino acid peptide sequence at a corresponding position identified in Table 1A; and
      wherein the glycan structure has a glycan composition; and/or
composition comprising a glycopeptide structure selected as one from a group consisting of peptide structures PS-3, PS-8, PS-9, or PS-20 to PS-35 identified in Table 1B, wherein:
the glycopeptide structure comprises:
   an amino acid peptide sequence identified in Table 5B as corresponding to the glycopeptide structure; and
   a glycan structure identified in Table 7B as corresponding to the glycopeptide structure in which the glycan structure is linked to a residue of the amino acid peptide sequence at a corresponding position identified in Table 1B; and
      wherein the glycan structure has a glycan composition.

46. The composition of embodiment 45, wherein the glycan composition is identified in Table 7A or Table 7B.

47. The composition of embodiment 45 or embodiment 46, wherein:
the glycopeptide structure has a precursor ion having a charge identified in Table 4A or Table 4B as corresponding to the glycopeptide structure.

48. The composition of any one of embodiments 45-47, wherein:
the glycopeptide structure has a precursor ion with an m/z ratio within ±1.5 of the m/z ratio listed for the precursor ion in Table 4 or Table 4B as corresponding to the glycopeptide structure.

49. The composition of any one of embodiments 45-47, wherein:
the glycopeptide structure has a precursor ion with an m/z ratio within ±1.0 of the m/z ratio listed for the precursor ion in Table 4A or Table 4B as corresponding to the glycopeptide structure.

50. The composition of any one of embodiments 45-47, wherein:
the glycopeptide structure has a precursor ion with an m/z ratio within ±0.5 of the m/z ratio listed for the precursor ion in Table 4A or Table 4B as corresponding to the glycopeptide structure.

51. The composition of any one of embodiments 45-50, wherein:
the glycopeptide structure has a product ion with an m/z ratio within ±1.0 of the m/z ratio listed for the first product ion in Table 4A or Table 4B as corresponding to the glycopeptide structure.

52. The composition of any one of embodiments 45-50, wherein:
the glycopeptide structure has a product ion with an m/z ratio within ±0.8 of the m/z ratio listed for the first product ion in Table 4A or Table 4B as corresponding to the glycopeptide structure.

53. The composition of any one of embodiments 45-50, wherein:
the glycopeptide structure has a product ion with an m/z ratio within ±0.5 of the m/z ratio listed for the first product ion in Table 4A or Table 4B as corresponding to the glycopeptide structure.

54. The composition of embodiment 50, wherein the glycopeptide structure has a monoisotopic mass identified in Table 1A or Table 1B as corresponding to the glycopeptide structure.

55. A composition comprising a peptide structure selected as one from a plurality of peptide structures identified in Table 1A, wherein:
the peptide structure has a monoisotopic mass identified as corresponding to the peptide structure in Table 1A; and
the peptide structure comprises the amino acid sequence of SEQ ID NOs: 14-28 identified in Table 1A as corresponding to the peptide structure; and/or
composition comprising a peptide structure selected as one from a plurality of peptide structures identified in Table 1B, wherein:
the peptide structure has a monoisotopic mass identified as corresponding to the peptide structure in Table 1B; and
the peptide structure comprises the amino acid sequence of SEQ ID NOs: 15, 20, or 41-53 identified in Table 1B as corresponding to the peptide structure.

56. The composition of embodiment 55, wherein:
the peptide structure has a precursor ion having a charge identified in Table 4A or 4B as corresponding to the peptide structure.

57. The composition of any one of embodiments 55-56, wherein:
the peptide structure has a precursor ion with an m/z ratio within ±1.5 of the m/z ratio listed for the precursor ion in Table 4A or 4B as corresponding to the peptide structure.

58. The composition of any one of embodiments 55-56, wherein:
the peptide structure has a precursor ion with an m/z ratio within ±1.0 of the m/z ratio listed for the precursor ion in Table 4A or 4B as corresponding to the peptide structure.

59. The composition of any one of embodiments 55-56, wherein:
the peptide structure has a precursor ion with an m/z ratio within ±0.5 of the m/z ratio listed for the precursor ion in Table 4A or 4B as corresponding to the peptide structure.

60. The composition of any one of embodiments 55-59, wherein:
the peptide structure has a product ion with an m/z ratio within ±1.0 of the m/z ratio listed for the first product ion in Table 4A or 4B as corresponding to the peptide structure.

61. The composition of any one of embodiments 55-59, wherein:
the peptide structure has a product ion with an m/z ratio within ±0.8 of the m/z ratio listed for the first product ion in Table 4A or 4B as corresponding to the peptide structure.

62. The composition of any one of embodiments 55-59, wherein:
the peptide structure has a product ion with an m/z ratio within ±0.5 of the m/z ratio listed for the first product ion in Table 4A or 4B as corresponding to the peptide structure.

63. A kit comprising at least one agent for quantifying at least one peptide structure identified in Table 1A and/or Table 1B to carry out the method of any one of embodiments 1-42.

64. A kit comprising at least one of a glycopeptide standard, a buffer, or a set of peptide sequences to carry out the method of any one of embodiments 1-42, a peptide sequence of the set of peptide sequences identified by a corresponding one of SEQ ID NOS: 14-28, defined in Table 1A and/or a kit comprising at least one of a glycopeptide standard, a buffer, or a set of peptide sequences to carry out the method of any one of embodiments 1-42, a peptide sequence of the set of peptide sequences identified by a corresponding one of SEQ ID NOS: 15, 20, or 41-53, defined in Table 1B.

65. A system comprising:
one or more data processors; and
a non-transitory computer readable storage medium containing instructions which, when executed on the one or more data processors, cause the one or more data processors to perform part or all of any one of embodiments 1-42.

66. A computer-program product tangibly embodied in a non-transitory machine-readable storage medium, including instructions configured to cause one or more data processors to perform part or all of any one of embodiments 1-42.

67. The method of embodiment 6, wherein the glycan structure corresponds to a glycan structure GL number in accordance with Table 1A or 1B, wherein the glycan structure comprises a symbol structure in accordance with the glycan structure GL number of Tables 1A or 1B and Table 7A or 7B.

68. The method of embodiment 6, wherein the glycan structure corresponds to a glycan structure GL number in accordance with Table 1A or 1B, wherein the glycan structure comprises a glycan composition in accordance with the glycan structure GL number of Tables 1A or 1B and Table 7A or 7B.

### XI. Additional Considerations

Any headers and/or sub-headers between sections and subsections of this document are included solely for the purpose of improving readability and do not imply that features cannot be combined across sections and subsection. Accordingly, sections and subsections do not describe separate embodiments.

While the present teachings are described in conjunction with various embodiments, it is not intended that the present teachings be limited to such embodiments. On the contrary, the present teachings encompass various alternatives, modifications, and equivalents, as will be appreciated by those of skill in the art. The present description provides preferred exemplary embodiments, and is not intended to limit the scope, applicability or configuration of the disclosure. Rather, the present description of the preferred exemplary embodiments will provide those skilled in the art with an enabling description for implementing various embodiments.

It is understood that various changes may be made in the function and arrangement of elements without departing from the spirit and scope as set forth in the appended claims. Thus, such modifications and variations are considered to be within the scope set forth in the appended claims. Further, the terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed.

In describing the various embodiments, the specification may have presented a method and/or process as a particular sequence of steps. However, to the extent that the method or process does not rely on the particular order of steps set forth herein, the method or process should not be limited to the particular sequence of steps described, and one skilled in the art can readily appreciate that the sequences may be varied and still remain within the spirit and scope of the various embodiments.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

Specific details are given in the present description to provide an understanding of the embodiments. However, it is understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

1. A method for diagnosing a subject with respect to a nasopharyngeal carcinoma (NPC) disease state, the method comprising:
receiving peptide structure data corresponding to a biological sample obtained from the subject;
analyzing the peptide structure data using a supervised machine learning model to generate a disease indicator that indicates whether biological sample evidences the NPC disease state based on at least 3 peptide structures selected from a group of peptide structures identified in Table 1A and/or Table 1B,
wherein the group of peptide structures in Table 1A and/or Table 1B is associated with the NPC disease state; and
wherein the group of peptide structures is listed in Table 1A and/or Table 1B with respect to relative significance to the disease indicator; and
generating a diagnosis output based on the disease indicator.

2. The method of claim 1, wherein the disease indicator comprises a score.

3. The method of claim 2, wherein generating the diagnosis output comprises:
determining that the score falls above a selected threshold; and
generating the diagnosis output based on the score falling above the selected threshold, wherein the diagnosis output includes a positive diagnosis for the NPC state.

4. The method of claim 2, wherein generating the diagnosis output comprises:
determining that the score falls below a selected threshold; and
generating the diagnosis output based on the score falling below the selected threshold, wherein the diagnosis output includes a negative diagnosis for the NPC state.

5. The method of claim 1, wherein analyzing the peptide structure data comprises:
analyzing the peptide structure data using a regression model.

6. The method of claim 1, wherein the at least one peptide structure comprises a glycopeptide structure defined by a peptide sequence and a glycan structure linked to the peptide sequence at a linking site of the peptide sequence, as identified in Table 1A, with the peptide sequence being one of SEQ ID NOS: 14-28 as defined in Table 1A and/or as identified in Table 1B, with the peptide sequence being one of SEQ ID NOS: 15, 20, or 41-53.

7. The method of claim 1, further comprising:
training the supervised machine learning model using training data,
wherein the training data comprises a plurality of peptide structure profiles for a plurality of subjects and a plurality of diagnoses for the plurality of subjects.

8. The method of claim 7, further comprising:
performing a differential expression analysis using initial training data to compare a first portion of the plurality of subjects diagnosed with the NPC disease state versus a second portion of the plurality of subjects diagnosed with a non-NPC state; and
identifying a training group of peptide structures based on the differential expression analysis for use as prognostic markers for the NPC disease state; and
forming the training data based on the training group of peptide structures identified.

9. The method of claim 8, wherein the non-NPC state includes at least one of a healthy state or a control state.

10. The method of claim 7, wherein training the supervised machine learning model using the training data includes reducing the training group of peptide structures to the group of peptide structures identified in Table 1A and/or Table 1B.

11. The method of claim 1, wherein the machine learning model comprises a logistic regression model.

12. The method of claim 1, wherein the quantification data for a peptide structure of the set of peptide structures comprises at least one of an abundance, a relative abundance, a normalized abundance, a relative quantity, an adjusted quantity, a normalized quantity, a relative concentration, an adjusted concentration, or a normalized concentration.

13. The method of claim 1, wherein the peptide structure data is generated using multiple reaction monitoring mass spectrometry (MRM-MS).

14. The method of claim 1, further comprising:
creating a sample from the biological sample; and
preparing the sample using reduction, alkylation, and enzymatic digestion to form a prepared sample that includes a set of peptide structures.

15. The method of claim 14, further comprising:
generating the peptide structure data from the prepared sample using multiple reaction monitoring mass spectrometry (MRM-MS).

16. The method of claim 1, wherein generating the diagnosis output comprises:
generating a report that identifies that the biological sample evidences the NPC disease state.

17. The method of claim 1, further comprising:
generating a treatment output based on at least one of the diagnosis output or the disease indicator.

18. The method of claim 17, wherein the treatment output comprises at least one of an identification of a treatment to treat the subject and a treatment schedule; and
the treatment comprises at least one of radiation therapy, chemoradiotherapy, surgery, or a targeted drug therapy.

19.-36. (canceled)

37. A method of monitoring a subject for a nasopharyngeal carcinoma (NPC), the method comprising:
receiving peptide structure data of a first biological sample obtained from a subject at a first timepoint;
analyzing the peptide structure data of the first biological sample using a supervised machine learning model to generate a first disease indicator based on at least 3 peptide structures selected from a group of peptide structures identified in Table 1A and/or Table 1B,
wherein the group of peptide structures in Table 1A and/or Table 1B comprises a group of peptide structures associated with the NPC disease state;
receiving peptide structure data of a second biological sample obtained from the subject at a second timepoint;
analyzing the peptide structure data of the second biological sample using the supervised machine learning model to generate a second disease indicator based on the at least 3 peptide structures selected from the group of peptide structures identified in Table 1A and/or Table 1B; and
generating a diagnosis output based on the first disease indicator and the second disease indicator.

38.-41. (canceled)

42. The method of any one of claims 37-41, further comprising treating the subject after the first biological sample is obtained and before the second biological sample is obtained.

43.-68. (canceled)
